(19) United States
(12) Patent Application Publication
(10) Pub. No.: US 2024/0150291 A1
(43) Pub. Date: May 9, 2024

(54) PROCESS FOR THE PREPARATION OF NLRP3 INHIBITORS

(71) Applicant: Hoffmann-La Roche Inc., Little Falls, NJ (US)

(72) Inventors: Paul FRASER, Abingdon (GB); Jetta PALGUNA, Hyderabad (IN); Mallesh BHARATHA, Hyderabad (IN); Joséphine Eliette Françoise CINQUALBRE, Saint-Louis (FR); Régis Jean Georges MONDIÈRE, Waltenheim (FR); Paolo TOSATTI, Binningen (CH)

(73) Assignee: Hoffmann-La Roche Inc., Little Falls, NJ (US)

(21) Appl. No.: 18/545,711

(22) Filed: Dec. 19, 2023

Related U.S. Application Data
(63) Continuation of application No. PCT/EP2022/067126, filed on Jun. 23, 2022.

(30) Foreign Application Priority Data
Jun. 23, 2021 (IN) 202141028180

(51) Int. Cl. C07D 211/54

(52) U.S. Cl. CPC C07D 211/54

(57) ABSTRACT

The present invention relates to intermediates and processes useful for preparing 1-ethyl-N-((1,2,3,5,6,7-hexahydro-s-indacen-4-yl)carbamoyl)piperidine-4-sulfonamide and salts thereof. The present invention further relates to 1-ethyl-N-((1,2,3,5,6,7-hexahydro-s-indacen-4-yl)carbamoyl)piperidine-4-sulfonamide and salts thereof when prepared by such processes and to associated pharmaceutical compositions and uses for the treatment and prevention of medical disorders and diseases, most especially by NLRP3 inhibition.

### FIELD OF THE INVENTION

The present invention relates to intermediates and processes useful for preparing 1-ethyl-N-((1,2,3,5,6,7-hexahydro-s-indacen-4-yl)carbamoyl)piperidine-4-sulfonamide and salts thereof. The present invention further relates to 1-ethyl-N-((1,2,3,5,6,7-hexahydro-s-indacen-4-yl)carbamoyl)piperidine-4-sulfonamide and salts thereof when prepared by such processes and to associated pharmaceutical compositions and uses for the treatment and prevention of medical disorders and diseases, most especially by NLRP3 inhibition.

### BACKGROUND

1-Ethyl-N-((1,2,3,5,6,7-hexahydro-s-indacen-4-yl)carbamoyl)piperidine-4-sulfonamide is disclosed in WO 2019/008025 A1 as an NLRP3 inhibitor (see Example 6). However, there is a need to provide improved processes for preparing 1-ethyl-N-((1,2,3,5,6,7-hexahydro-s-indacen-4-yl)carbamoyl)piperidine-4-sulfonamide and salts thereof. In particular, there is a need to provide efficient processes that are suitable for large scale synthesis and which, for example, avoid costly chromatographic or high temperature techniques, avoid or minimise the use of expensive reagents, and/or avoid the generation of hazardous by-products. There is also a need to provide 1-ethyl-N-((1,2,3,5,6,7-hexahydro-s-indacen-4-yl)carbamoyl)piperidine-4-sulfonamide and salts thereof at a higher yield and/or with a higher purity compared to prior art processes, especially on a large scale. The present invention solves the aforementioned problems.

### SUMMARY OF THE INVENTION

A first aspect of the invention provides a process of preparing 1-ethyl-N-((1,2,3,5,6,7-hexahydro-s-indacen-4-yl)carbamoyl)piperidine-4-sulfonamide or a salt thereof, comprising the step of contacting 1-ethyl-4-piperidinesulfonamide (A) with a 1,2,3,5,6,7-hexahydro-s-indacene derivative (B) in the presence of a solvent to obtain 1-ethyl-N-((1,2,3,5,6,7-hexahydro-s-indacen-4-yl)carbamoyl)piperidine-4-sulfonamide (C) or a salt thereof:

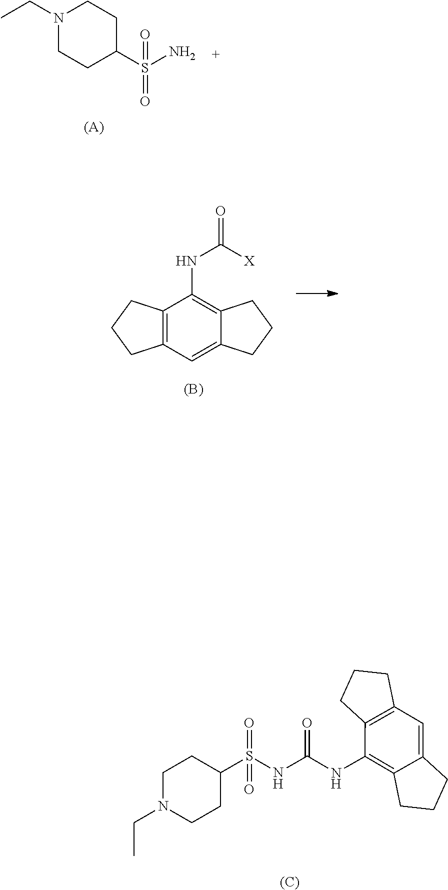

wherein X is a leaving group.

In one embodiment of the first aspect of the invention, X is Cl, Br, I, OR¹, SR¹, N(R¹)₂, OP(═O)(R¹)₂ or OP(R¹)₃⁺, wherein each R¹ is independently selected from a C₁-C₂₀ hydrocarbyl group, wherein each C₁-C₂₀ hydrocarbyl group may be straight-chained or branched, or be or include one or more cyclic groups, wherein each C₁-C₂₀ hydrocarbyl group may optionally be substituted with one or more oxo (═O) and/or one or more halo groups, and wherein each C₁-C₂₀ hydrocarbyl group may optionally include one or more heteroatoms independently selected from N, O and S in its carbon skeleton, or wherein any two R¹ together with the nitrogen or phosphorus atom to which they are attached may form a 3- to 16-membered heterocyclic group, wherein the heterocyclic group may be monocyclic, bicyclic or tricyclic, and wherein the heterocyclic group may optionally be substituted with one or more halo groups and/or one or more groups R^{X}, wherein each R^{X} is independently selected from a —CN, —OH, —NH₂, oxo (═O), ═NH or C₁-C₆ hydrocarbyl group, wherein each C₁-C₆ hydrocarbyl group may be straight-chained or branched, or be or include one or more cyclic groups, wherein each C₁-C₆ hydrocarbyl group may optionally be substituted with one or more halo groups, and wherein each C₁-C₆ hydrocarbyl group may optionally include one or more heteroatoms independently selected from N, O and S in its carbon skeleton.

In the context of the present specification, a “hydrocarbyl” substituent group or a hydrocarbyl moiety in a substituent group only includes carbon and hydrogen atoms but, unless stated otherwise, does not include any heteroatoms, such as N, O or S, in its carbon skeleton. A hydrocarbyl group/moiety may be saturated or unsaturated (including aromatic), and may be straight-chained or branched, or be or include cyclic groups wherein, unless stated otherwise, the cyclic group does not include any heteroatoms, such as N, O or S, in its carbon skeleton. Examples of hydrocarbyl groups include alkyl, alkenyl, alkynyl, cycloalkyl, cycloalkenyl and aryl groups/moieties and combinations of all of these groups/moieties. Typically a hydrocarbyl group is a C₁-C₂₀ hydrocarbyl group. More typically a hydrocarbyl group is a C₁-C₁₅ hydrocarbyl group. More typically a hydrocarbyl group is a C₁-C₁₀ hydrocarbyl group. A “hydrocarbylene” group is similarly defined as a divalent hydrocarbyl group.

An “alkyl” substituent group or an alkyl moiety in a substituent group may be linear (i.e. straight-chained) or branched. Examples of alkyl groups/moieties include methyl, ethyl, n-propyl, i-propyl, n-butyl, i-butyl, t-butyl and n-pentyl groups/moieties. Unless stated otherwise, the term “alkyl” does not include “cycloalkyl”. Typically an alkyl group is a C₁-C₁₂ alkyl group. More typically an alkyl group is a C₁-C₆ alkyl group. An “alkylene” group is similarly defined as a divalent alkyl group.

An “alkenyl” substituent group or an alkenyl moiety in a substituent group refers to an unsaturated alkyl group or moiety having one or more carbon-carbon double bonds. Examples of alkenyl groups/moieties include ethenyl, propenyl, 1-butenyl, 2-butenyl, 1-pentenyl, 1-hexenyl, 1,3-butadienyl, 1,3-pentadienyl, 1,4-pentadienyl and 1,4-hexadienyl groups/moieties. Unless stated otherwise, the term “alkenyl” does not include “cycloalkenyl”. Typically an alkenyl group is a C₂-C₁₂ alkenyl group. More typically an alkenyl group is a C₂-C₆ alkenyl group. An “alkenylene” group is similarly defined as a divalent alkenyl group.

An “alkynyl” substituent group or an alkynyl moiety in a substituent group refers to an unsaturated alkyl group or moiety having one or more carbon-carbon triple bonds. Examples of alkynyl groups/moieties include ethynyl, propargyl, but-1-ynyl and but-2-ynyl groups/moieties. Typically an alkynyl group is a C₂-C₁₂ alkynyl group. More typically an alkynyl group is a C₂-C₆ alkynyl group. An “alkynylene” group is similarly defined as a divalent alkynyl group.

A “cyclic” substituent group or a cyclic moiety in a substituent group refers to any hydrocarbyl ring, wherein the hydrocarbyl ring may be saturated or unsaturated (including aromatic) and may include one or more heteroatoms, e.g. N, O or S, in its carbon skeleton. Examples of cyclic groups include cycloalkyl, cycloalkenyl, heterocyclic, aryl and heteroaryl groups as discussed below. A cyclic group may be monocyclic, bicyclic (e.g. bridged, fused or spiro), or polycyclic. Typically, a cyclic group is a 3- to 12-membered cyclic group, which means it contains from 3 to 12 ring atoms. More typically, a cyclic group is a 3- to 7-membered monocyclic group, which means it contains from 3 to 7 ring atoms.

A “heterocyclic” substituent group or a heterocyclic moiety in a substituent group refers to a cyclic group or moiety including one or more carbon atoms and one or more (such as one, two, three or four) heteroatoms, e.g. N, O or S, in the ring structure. Examples of heterocyclic groups include heteroaryl groups as discussed below and non-aromatic heterocyclic groups such as azetinyl, azetidinyl, oxetanyl, thietanyl, pyrrolidinyl, tetrahydrofuranyl, tetrahydrothiophenyl, pyrazolidinyl, imidazolidinyl, dioxolanyl, oxathiolanyl, piperidinyl, tetrahydropyranyl, thianyl, piperazinyl, dioxanyl, morpholinyl and thiomorpholinyl groups.

A “cycloalkyl” substituent group or a cycloalkyl moiety in a substituent group refers to a saturated hydrocarbyl ring containing, for example, from 3 to 7 carbon atoms, examples of which include cyclopropyl, cyclobutyl, cyclopentyl and cyclohexyl. Unless stated otherwise, a cycloalkyl substituent group or moiety may include monocyclic, bicyclic or polycyclic hydrocarbyl rings.

A “cycloalkenyl” substituent group or a cycloalkenyl moiety in a substituent group refers to a non-aromatic unsaturated hydrocarbyl ring having one or more carbon-carbon double bonds and containing, for example, from 3 to 7 carbon atoms, examples of which include cyclopent-1-en-1-yl, cyclohex-1-en-1-yl and cyclohex-1,3-dien-1-yl. Unless stated otherwise, a cycloalkenyl substituent group or moiety may include monocyclic, bicyclic or polycyclic hydrocarbyl rings.

An “aryl” substituent group or an aryl moiety in a substituent group refers to an aromatic hydrocarbyl ring. The term “aryl” includes monocyclic aromatic hydrocarbons and polycyclic fused ring aromatic hydrocarbons wherein all of the fused ring systems (excluding any ring systems which are part of or formed by optional substituents) are aromatic. Examples of aryl groups/moieties include phenyl, naphthyl, anthracenyl and phenanthrenyl. Unless stated otherwise, the term “aryl” does not include “heteroaryl”.

A “heteroaryl” substituent group or a heteroaryl moiety in a substituent group refers to an aromatic heterocyclic group or moiety. The term “heteroaryl” includes monocyclic aromatic heterocycles and polycyclic fused ring aromatic heterocycles wherein all of the fused ring systems (excluding any ring systems which are part of or formed by optional substituents) are aromatic. Examples of heteroaryl groups/moieties include the following:

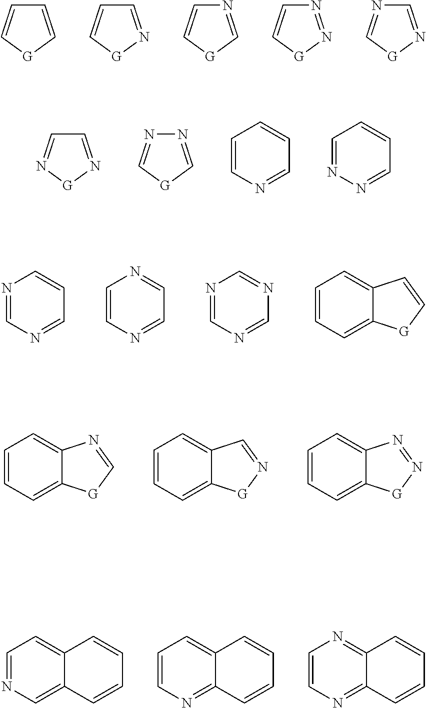

wherein G=O, S or NH.

For the purposes of the present specification, where a combination of moieties is referred to as one group, for example, arylalkyl, arylalkenyl, arylalkynyl, alkylaryl, alkenylaryl or alkynylaryl, the last mentioned moiety contains the atom by which the group is attached to the rest of the molecule. An example of an arylalkyl group is benzyl.

The term “halo” includes fluoro, chloro, bromo and iodo.

Unless stated otherwise, where a group is prefixed by the term “halo”, such as a haloalkyl or halomethyl group, it is to be understood that the group in question is substituted with one or more halo groups independently selected from fluoro, chloro, bromo and iodo. Typically, the maximum number of halo substituents is limited only by the number of hydrogen atoms available for substitution on the corresponding group without the halo prefix. For example, a halomethyl group may contain one, two or three halo substituents. A haloethyl or halophenyl group may contain one, two, three, four or five halo substituents. Similarly, unless stated otherwise, where a group is prefixed by a specific halo group, it is to be understood that the group in question is substituted with one or more of the specific halo groups. For example, the term “fluoromethyl” refers to a methyl group substituted with one, two or three fluoro groups.

Similarly, unless stated otherwise, where a group is said to be “halo-substituted”, it is to be understood that the group in question is substituted with one or more halo groups independently selected from fluoro, chloro, bromo and iodo. Typically, the maximum number of halo substituents is limited only by the number of hydrogen atoms available for substitution on the group said to be halo-substituted. For example, a halo-substituted methyl group may contain one, two or three halo substituents. A halo-substituted ethyl or halo-substituted phenyl group may contain one, two, three, four or five halo substituents.

Unless stated otherwise, any reference to an element is to be considered a reference to all isotopes of that element. Thus, for example, unless stated otherwise any reference to hydrogen is considered to encompass all isotopes of hydrogen including deuterium and tritium.

Unless stated otherwise, any reference to a compound or group is to be considered a reference to all tautomers of that compound or group.

Where reference is made to a hydrocarbyl or other group including one or more heteroatoms N, O or S in its carbon skeleton, or where reference is made to a carbon atom of a hydrocarbyl or other group being replaced by an N, O or S atom, what is intended is that:

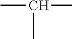

is replaced by

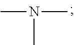

—CH₃ is replaced by —NH₂, —OH or —SH;
—CH═ is replaced by —N═;
CH₂═ is replaced by NH═, O═ or S═; or
CH≡ is replaced by N≡;
   provided that the resultant group comprises at least one carbon atom. For example, methoxy, dimethylamino and aminoethyl groups are considered to be hydrocarbyl groups including one or more heteroatoms N, O or S in their carbon skeleton.

As used herein, where it is stated that a group such as a hydrocarbyl group is substituted with an oxo (═O) group, it is to be understood that any two hydrogen atoms attached to the same atom may be replaced by a π-bonded ═O substituent, or where the group contains a nitrogen or sulphur atom, the oxidation state of the nitrogen or sulphur atom may be changed so as to permit the attachment of a π-bonded ═O substituent, optionally with the loss of one or more hydrogen atoms from the nitrogen atom, the sulphur atom or a neighbouring atom to allow for charge neutralisation. Thus, for example, —CH₂CHO, —CH₂NO₂ and —CH₂SO₃H are examples of —CH₂CH₃, —CH₂NHOH and —CH₂—S—OH groups respectively substituted with one (—CH₂CHO, —CH₂NO₂) or two (—CH₂SO₃H) oxo groups.

In the context of the present specification, unless otherwise stated, a Cₓ-C_{y} group is defined as a group containing from x to y carbon atoms. For example, a C₁-C₄ alkyl group is defined as an alkyl group containing from 1 to 4 carbon atoms. Optional substituents and moieties are not taken into account when calculating the total number of carbon atoms in the parent group substituted with the optional substituents and/or containing the optional moieties. For the avoidance of doubt, replacement heteroatoms, e.g. N, O or S, are not to be counted as carbon atoms when calculating the number of carbon atoms in a Cₓ-C_{y} group. For example, a morpholinyl group is to be considered a C₄ heterocyclic group, not a C₆ heterocyclic group.

In one embodiment of the first aspect of the invention, X is Cl, Br or I. Typically in such an embodiment, X is Cl.

In another embodiment of the first aspect of the invention, X is OR¹ or SR¹, wherein R¹ is a C₁-C₂₀ hydrocarbyl group, wherein the C₁-C₂₀ hydrocarbyl group may be straight-chained or branched, or be or include one or more cyclic groups, wherein the C₁-C₂₀ hydrocarbyl group may optionally be substituted with one or more oxo (═O) and/or one or more halo groups, and wherein the C₁-C₂₀ hydrocarbyl group may optionally include one or more heteroatoms independently selected from N, O and S in its carbon skeleton.

Typically in such an embodiment, X is OR¹.

For example, X may be OR¹, wherein R¹ is selected from an alkyl, cycloalkyl, aryl, heteroaryl, arylalkyl or heteroarylalkyl group, wherein R¹ may optionally be substituted with one or more substituents independently selected from halo, —CN, —OH, —NO₂, —NH₂, oxo (═O), ═NH, —R¹⁰, —OR¹⁰—, —NHR¹⁰, —N(R¹⁰)₂, —N(O)(R¹⁰)₂, or ═NR¹⁰, wherein each R¹⁰ is independently selected from a C₁-C₄ alkyl, C₁-C₄ haloalkyl, C₃-C₄ cycloalkyl or C₃-C₄ halocycloalkyl group, or any two R¹⁰ directly attached to the same nitrogen atom may together form a C₂-C₅ alkylene or C₂-C₅ haloalkylene group, and wherein R¹, including any optional substituents, contains from 1 to 20 carbon atoms.

More typically, X is OR¹, wherein R¹ is selected from an alkyl, cycloalkyl, aryl, heteroaryl, arylalkyl or heteroarylalkyl group, wherein R¹ may optionally be substituted with one or more substituents independently selected from halo, —CN, —OH, —NO₂, —NH₂, oxo (═O), -Me, -Et, —OMe, —OEt, —NHMe, —NHEt, —N(Me)₂, —N(Me)Et or —N(Et)₂, wherein any methyl (Me) or ethyl (Et) group may optionally be substituted with one or more halo groups, and wherein R¹, including any optional substituents, contains from 1 to 12 carbon atoms.

In one embodiment, X is OR¹, wherein R¹ is selected from an aryl or heteroaryl group, wherein the aryl or heteroaryl group is monocyclic, bicyclic or tricyclic, wherein R¹ may optionally be substituted with one or more substituents independently selected from halo, —CN, —OH, —NO₂, —NH₂, —R¹⁰, —OR¹⁰, —NHR¹⁰, —N(R¹⁰)₂ or —N(O)(R¹⁰)₂, wherein each R¹⁰ is independently selected from a C₁-C₄ alkyl, C₁-C₄ haloalkyl, C₃-C₄ cycloalkyl or C₃-C₄ halocycloalkyl group, or any two R¹⁰ directly attached to the same nitrogen atom may together form a C₂-C₅ alkylene or C₂-C₅ haloalkylene group, and wherein R¹, including any optional substituents, contains from 1 to 20 carbon atoms.

More typically, X is OR¹, wherein R¹ is selected from a phenyl or a monocyclic heteroaryl group, wherein R¹ may optionally be substituted with one or more substituents independently selected from halo, —CN, —OH, —NO₂, —NH₂, -Me, -Et, —OMe, —OEt, —NHMe, —NHEt, —N(Me)₂, —N(Me)Et or —N(Et)₂, wherein any methyl (Me) or ethyl (Et) group may optionally be substituted with one or more halo groups, and wherein R¹, including any optional substituents, contains from 1 to 12 carbon atoms.

More typically still, X is OR¹, wherein R¹ is a phenyl group, wherein the phenyl group is optionally substituted with one or more fluoro, chloro or —NO₂ groups. Most typically, R¹ is an unsubstituted phenyl group, i.e. X is OPh.

When R¹ is an unsubstituted phenyl group, there is provided a process of preparing 1-ethyl-N-((1,2,3,5,6,7-hexahydro-s-indacen-4-yl)carbamoyl)piperidine-4-sulfonamide or a salt thereof, comprising the step of contacting 1-ethyl-4-piperidinesulfonamide (A) with 4-(phenoxycarbonylamino)-1,2,3,5,6,7-hexahydro-s-indacene (B′) in the presence of a solvent to obtain 1-ethyl-N-((1,2,3,5,6,7-hexahydro-s-indacen-4-yl)carbamoyl)-piperidine-4-sulfonamide (C) or a salt thereof:

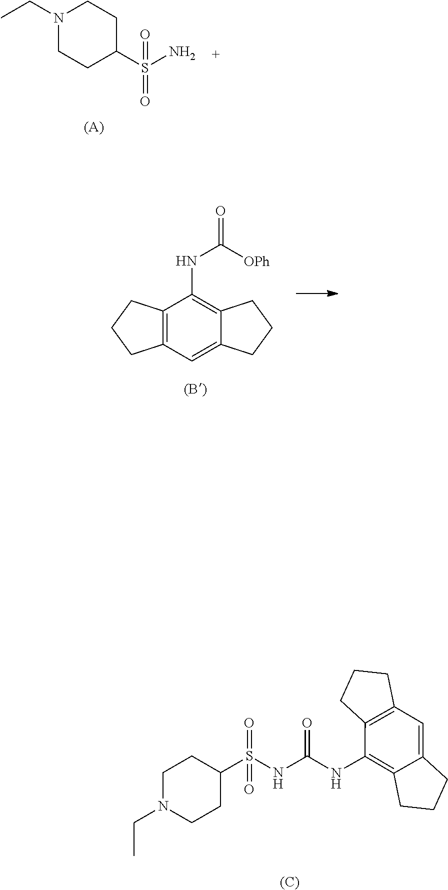

In another embodiment of the first aspect of the invention, X is N(R¹)₂, wherein each R¹ is independently selected from a C₁-C₂₀ hydrocarbyl group, wherein each C₁-C₂₀ hydrocarbyl group may be straight-chained or branched, or be or include one or more cyclic groups, wherein each C₁-C₂₀ hydrocarbyl group may optionally be substituted with one or more oxo (═O) and/or one or more halo groups, and wherein each C₁-C₂₀ hydrocarbyl group may optionally include one or more heteroatoms independently selected from N, O and S in its carbon skeleton, or wherein any two R¹ together with the nitrogen atom to which they are attached may form a 3- to 16-membered heterocyclic group, wherein the heterocyclic group may be monocyclic, bicyclic or tricyclic, and wherein the heterocyclic group may optionally be substituted with one or more halo groups and/or one or more groups R^{X}, wherein each R^{X} is independently selected from a —CN, —OH, —NH₂, oxo (═O), ═NH or C₁-C₆ hydrocarbyl group, wherein each C₁-C₆ hydrocarbyl group may be straight-chained or branched, or be or include one or more cyclic groups, wherein each C₁-C₆ hydrocarbyl group may optionally be substituted with one or more halo groups, and wherein each C₁-C₆ hydrocarbyl group may optionally include one or more heteroatoms independently selected from N, O and S in its carbon skeleton.

Typically in such an embodiment, X is N(R¹)₂, wherein the two R¹ together with the nitrogen atom to which they are attached form a 5- to 14-membered heteroaryl group, wherein the heteroaryl group may be monocyclic, bicyclic or tricyclic, wherein R¹ may optionally be substituted with one or more substituents independently selected from halo, —CN, —OH, —NO₂, —NH₂, —R¹⁰, —OR¹⁰—, —NHR¹⁰, —N(R¹⁰)₂ or —N(O)(R¹⁰)₂, wherein each R¹⁰ is independently selected from a C₁-C₄ alkyl, C₁-C₄ haloalkyl, C₃-C₄ cycloalkyl or C₃-C₄ halocycloalkyl group, or any two R¹⁰ directly attached to the same nitrogen atom may together form a C₂-C₅ alkylene or C₂-C₅ haloalkylene group, and wherein R¹, including any optional substituents, contains from 1 to 20 carbon atoms.

More typically, where X is N(R¹)₂, the two R¹ together with the nitrogen atom to which they are attached form a 5- to 10-membered heteroaryl group, wherein the heteroaryl group may be monocyclic or bicyclic, wherein R¹ may optionally be substituted with one or more substituents independently selected from halo, —CN, —OH, —NO₂, —NH₂, -Me, -Et, —OMe, —OEt, —NHMe, —NHEt, —N(Me)₂, —N(Me)Et or —N(Et)₂, wherein any methyl (Me) or ethyl (Et) group may optionally be substituted with one or more halo groups, and wherein R¹, including any optional substituents, contains from 1 to 12 carbon atoms.

Typically, where X is N(R¹)₂ and the two R¹ together with the nitrogen atom to which they are attached form a 5- to 14- or 5- to 10-membered heteroaryl group, the ring that encompasses the nitrogen atom of N(R¹)₂ is a 5-membered ring.

In another embodiment of the first aspect of the invention, X is OP(═O)(R¹)₂ or OP(R¹)₃⁺, wherein each R¹ is independently selected from a C₁-C₂₀ hydrocarbyl group, wherein each C₁-C₂₀ hydrocarbyl group may be straight-chained or branched, or be or include one or more cyclic groups, wherein each C₁-C₂₀ hydrocarbyl group may optionally be substituted with one or more oxo (═O) and/or one or more halo groups, and wherein each C₁-C₂₀ hydrocarbyl group may optionally include one or more heteroatoms independently selected from N, O and S in its carbon skeleton, or wherein any two R¹ together with the phosphorus atom to which they are attached may form a 3- to 16-membered heterocyclic group, wherein the heterocyclic group may be monocyclic, bicyclic or tricyclic, and wherein the heterocyclic group may optionally be substituted with one or more halo groups and/or one or more groups R^{X}, wherein each R^{X} is independently selected from a —CN, —OH, —NH₂, oxo (═O), ═NH or C₁-C₆ hydrocarbyl group, wherein each C₁-C₆ hydrocarbyl group may be straight-chained or branched, or be or include one or more cyclic groups, wherein each C₁-C₆ hydrocarbyl group may optionally be substituted with one or more halo groups, and wherein each C₁-C₆ hydrocarbyl group may optionally include one or more heteroatoms independently selected from N, O and S in its carbon skeleton.

Typically in such an embodiment, X is OP(═O)(R¹)₂ or OP(R¹)₃⁺, wherein each R¹ is independently selected from an alkyl, cycloalkyl, aryl, heteroaryl, arylalkyl or heteroarylalkyl group, wherein each R¹ may optionally be substituted with one or more substituents independently selected from halo, —CN, —OH, —NO₂, —NH₂, oxo (═O), ═NH, —R¹⁰, —OR¹⁰, —NHR¹⁰, —N(R¹⁰)₂, —N(O)(R¹⁰)₂, or ═NR¹⁰, wherein each R¹⁰ is independently selected from a C₁-C₄ alkyl, C₁-C₄ haloalkyl, C₃-C₄ cycloalkyl or C₃-C₄ halocycloalkyl group, or any two R¹⁰ directly attached to the same nitrogen atom may together form a C₂-C₅ alkylene or C₂-C₅ haloalkylene group, and wherein each R¹, including any optional substituents, contains from 1 to 20 carbon atoms.

More typically, where X is OP(═O)(R¹)₂ or OP(R¹)₃⁺, each R¹ is independently selected from an alkyl, cycloalkyl, aryl, heteroaryl, arylalkyl or heteroarylalkyl group, wherein each R¹ may optionally be substituted with one or more substituents independently selected from halo, —CN, —OH, —NO₂, —NH₂, oxo (═O), -Me, -Et, —OMe, —OEt, —NHMe, —NHEt, —N(Me)₂, —N(Me)Et or —N(Et)₂, wherein any methyl (Me) or ethyl (Et) group may optionally be substituted with one or more halo groups, and wherein each R¹, including any optional substituents, contains from 1 to 12 carbon atoms.

More typically still, where X is OP(═O)(R¹)₂ or OP(R¹)₃⁺, each R¹ is independently selected from a C₁-C₄ alkyl or phenyl group.

In one embodiment of the first aspect of the invention, the solvent is a polar aprotic solvent such as dimethyl sulfoxide, N,N-dimethylformamide, N,N′-dimethylpropyleneurea, tetrahydrofuran, 1,4-dioxane, ethyl acetate, acetone, acetonitrile, dichloromethane, hexamethylphosphoramide, nitromethane, propylene carbonate, N-methyl pyrrolidone, or a mixture thereof. Typically the solvent does not comprise an ester. More typically the solvent does not comprise a carbonyl group. Typically the solvent is not halogenated. For example, the solvent may be selected from dimethyl sulfoxide, tetrahydrofuran, 1,4-dioxane, acetonitrile, hexamethylphosphoramide, nitromethane, or a mixture thereof. More typically still, the solvent does not comprise a carbonyl, C═N or C≡N group. Typically, where the solvent does not comprise a carbonyl, C═N or C≡N group, the solvent is not halogenated. For example, the solvent may be selected from dimethyl sulfoxide, tetrahydrofuran, 1,4-dioxane, hexamethylphosphoramide, nitromethane, or a mixture thereof. Most typically, the solvent is dimethyl sulfoxide.

In one embodiment of the first aspect of the invention, the step of contacting 1-ethyl-4-piperidinesulfonamide (A) with the 1,2,3,5,6,7-hexahydro-s-indacene derivative (B) or (B′) is performed in the presence of a base. Typically the base is an alkoxide base, such as an alkali metal or an alkali earth metal alkoxide. More typically the base is a tertiary butoxide base such as an alkali metal or an alkali earth metal tertiary butoxide.

Examples of suitable bases include potassium tertiary butoxide and sodium tertiary butoxide. Typically, the base is potassium tertiary butoxide.

One embodiment the first aspect of the invention provides a process of preparing a salt of 1-ethyl-N-((1,2,3,5,6,7-hexahydro-s-indacen-4-yl)carbamoyl)piperidine-4-sulfonamide, such as a cationic salt. Typically the salt is pharmaceutically acceptable.

For the purposes of this invention, a “cationic salt” of 1-ethyl-N-((1,2,3,5,6,7-hexahydro-s-indacen-4-yl)carbamoyl)piperidine-4-sulfonamide is a salt formed between a protic acid functionality (such as a urea proton) of the compound by the loss of a proton and a suitable cation. Suitable cations include, but are not limited to lithium, sodium, potassium, magnesium, calcium and ammonium. The salt may be a mono-, di-, tri- or multi-salt. Preferably the salt is a mono- or di-lithium, sodium, potassium, magnesium, calcium or ammonium salt. More preferably the salt is a mono- or di-sodium salt or a mono- or di-potassium salt. More preferably the salt is a mono- or di-potassium salt, more preferably still the salt is a mono-potassium salt.

Advantageously, where a cationic salt of 1-ethyl-N-((1,2,3,5,6,7-hexahydro-s-indacen-4-yl)carbamoyl)piperidine-4-sulfonamide (C) is desired, the cation of the salt is provided by the conjugate acid of the base. For example, one embodiment of the first aspect of the invention provides a process of preparing an alkali metal or an alkali earth metal salt of 1-ethyl-N-((1,2,3,5,6,7-hexahydro-s-indacen-4-yl)carbamoyl)piperidine-4-sulfonamide (C), comprising the step of contacting 1-ethyl-4-piperidinesulfonamide (A) with a 1,2,3,5,6,7-hexahydro-s-indacene derivative (B) or (B′) in the presence of a solvent and an alkali metal or an alkali earth metal alkoxide, to obtain the alkali metal or alkali earth metal salt of 1-ethyl-N-((1,2,3,5,6,7-hexahydro-s-indacen-4-yl)-carbamoyl)-piperidine-4-sulfonamide, wherein the alkali metal or alkali earth metal of the salt is the same as the alkali metal or alkali earth metal of the alkoxide. Typically in such an embodiment, the alkali metal or alkali earth metal alkoxide is an alkali metal or an alkali earth metal tertiary butoxide.

A further embodiment of the first aspect of the invention provides a process of preparing a potassium salt of 1-ethyl-N-((1,2,3,5,6,7-hexahydro-s-indacen-4-yl)carbamoyl)piperidine-4-sulfonamide (C), comprising the step of contacting 1-ethyl-4-piperidinesulfonamide (A) with 4-(phenoxycarbonylamino)-1,2,3,5,6,7-hexahydro-s-indacene (B′) in the presence of a solvent and potassium tertiary butoxide, to obtain the potassium salt of 1-ethyl-N-((1,2,3,5,6,7-hexahydro-s-indacen-4-yl)carbamoyl)-piperidine-4-sulfonamide. Typically in such an embodiment, the potassium salt is a mono-potassium salt.

In one embodiment of the first aspect of the invention, the step of contacting 1-ethyl-4-piperidinesulfonamide (A) with the 1,2,3,5,6,7-hexahydro-s-indacene derivative (B) or (B′) to obtain 1-ethyl-N-((1,2,3,5,6,7-hexahydro-s-indacen-4-yl)carbamoyl)piperidine-4-sulfonamide (C) or a salt thereof is carried out at a temperature in the range from −10 to 60° C. Typically, the step is carried out at a temperature in the range from 0 to 50° C., more typically in the range from 10 to 40° C., and most typically in the range from 20 to 30° C.

Typically in accordance with the first aspect of the invention, 1-ethyl-4-piperidine-sulfonamide (A) is present in or added to the solvent at an initial concentration of from 0.1 to 15 mol/L, relative to the total volume of solvent used in the reaction mixture. More typically, 1-ethyl-4-piperidinesulfonamide (A) is present in or added to the solvent at an initial concentration of from 0.5 to 5.0 mol/L. Most typically, 1-ethyl-4-piperidinesulfonamide (A) is present in or added to the solvent at an initial concentration of from 1.0 to 1.5 mol/L.

Typically in accordance with the first aspect of the invention, the 1,2,3,5,6,7-hexahydro-s-indacene derivative (B) or (B′) is present in or added to the solvent at an initial concentration of from 0.1 to 15 mol/L, relative to the total volume of solvent used in the reaction mixture. More typically, the 1,2,3,5,6,7-hexahydro-s-indacene derivative (B) or (B′) is present in or added to the solvent at an initial concentration of from 0.5 to 5.0 mol/L. Most typically, the 1,2,3,5,6,7-hexahydro-s-indacene derivative (B) or (B′) is present in or added to the solvent at an initial concentration of from 1.0 to 1.5 mol/L.

Typically, the process of the first aspect of the invention uses from 0.8 to 1.4 molar equivalents of the 1,2,3,5,6,7-hexahydro-s-indacene derivative (B) or (B′), relative to the initial amount of 1-ethyl-4-piperidinesulfonamide (A). More typically, the process uses from 1.0 to 1.2 molar equivalents of the 1,2,3,5,6,7-hexahydro-s-indacene derivative (B) or (B′). Most typically, the process uses from 1.05 to 1.15 molar equivalents of the 1,2,3,5,6,7-hexahydro-s-indacene derivative (B) or (B′).

Typically, where a base is employed, the process of the first aspect of the invention uses from 1.0 to 2.0 molar equivalents of the base, relative to the initial amount of 1-ethyl-4-piperidinesulfonamide (A). More typically, the process uses from 1.05 to 1.5 molar equivalents of the base. More typically still, the process uses from 1.1 to 1.2 molar equivalents of the base.

In one embodiment of the first aspect of the invention, the process comprises the steps of:
(i) dissolving the 1-ethyl-4-piperidinesulfonamide (A) in the solvent;
(ii) adding the base to the solution formed in step (i); and
(iii) adding the 1,2,3,5,6,7-hexahydro-s-indacene derivative (B) or (B′) to the mixture formed in step (ii).

In one embodiment of the first aspect of the invention, the 1-ethyl-N-((1,2,3,5,6,7-hexahydro-s-indacen-4-yl)carbamoyl)piperidine-4-sulfonamide (C) or the salt thereof is isolated from the reaction mixture by crystallisation or precipitation. For example, where the solvent used in the reaction is dimethyl sulfoxide (DMSO), further solvents such as water, acetonitrile (MeCN) and optionally further DMSO may be added to the reaction mixture to create a precipitation mixture from which the 1-ethyl-N-((1,2,3,5,6,7-hexahydro-s-indacen-4-yl)carbamoyl)piperidine-4-sulfonamide (C) or the salt thereof is precipitated, optionally under cooling. Typically, a salt of 1-ethyl-N-((1,2,3,5,6,7-hexahydro-s-indacen-4-yl)carbamoyl)piperidine-4-sulfonamide (C) is isolated from the reaction mixture by crystallisation or precipitation. Typically the salt is an alkali metal or alkali earth metal salt, such as a potassium salt.

In one embodiment of the first aspect of the invention, the precipitation mixture comprises DMSO, MeCN and water, wherein the solvent of the precipitation mixture consists of:
(i) 30-50 wt. % DMSO (relative to the total weight of the solvent);
(ii) 50-70 wt. % MeCN (relative to the total weight of the solvent); and
(iii) 1-10 wt. % H₂O (relative to the total weight of the solvent).

Typically the crystallisation or precipitation occurs at a temperature in the range from −10 to 20° C. More typically, the crystallisation or precipitation occurs at a temperature in the range from −5 to 10° C., and most typically in the range from 0 to 5° C.

In one embodiment of the first aspect of the invention, the salt of 1-ethyl-N-((1,2,3,5,6,7-hexahydro-s-indacen-4-yl)carbamoyl)piperidine-4-sulfonamide (C) is purified by recrystallisation or reprecipitation. For example, the crude salt of 1-ethyl-N-((1,2,3,5,6,7-hexahydro-s-indacen-4-yl)carbamoyl)piperidine-4-sulfonamide (C) may be dissolved in a first solvent to a obtain a first mixture, optionally the mixture may be filtered, and the salt of the 1-ethyl-N-((1,2,3,5,6,7-hexahydro-s-indacen-4-yl)-carbamoyl)piperidine-4-sulfonamide (C) may be precipitated by the addition of a second solvent, optionally with cooling. Typically, the first solvent is a polar protic solvent such as methanol. Typically, the second solvent is a polar aprotic solvent such as acetonitrile.

A second aspect of the invention provides 1-ethyl-N-((1,2,3,5,6,7-hexahydro-s-indacen-4-yl)carbamoyl)piperidine-4-sulfonamide or a salt thereof, prepared by or preparable by a process of the first aspect of this invention.

In one embodiment, the second aspect of the invention provides an alkali metal or an alkali earth metal salt of 1-ethyl-N-((1,2,3,5,6,7-hexahydro-s-indacen-4-yl)carbamoyl)-piperidine-4-sulfonamide. Typically, the second aspect of the invention provides a potassium salt of 1-ethyl-N-((1,2,3,5,6,7-hexahydro-s-indacen-4-yl)carbamoyl)-piperidine-4-sulfonamide. Most typically, the second aspect of the invention provides a mono-potassium salt of 1-ethyl-N-((1,2,3,5,6,7-hexahydro-s-indacen-4-yl)carbamoyl)-piperidine-4-sulfonamide.

In one embodiment of the second aspect of the invention, the 1-ethyl-N-((1,2,3,5,6,7-hexahydro-s-indacen-4-yl)carbamoyl)piperidine-4-sulfonamide or the salt thereof has a purity as measured by ¹H NMR of ≥97.0%. More typically, the 1-ethyl-N-((1,2,3,5,6,7-hexahydro-s-indacen-4-yl)carbamoyl)piperidine-4-sulfonamide or the salt thereof has a purity as measured by ¹H NMR of ≥98.0%, or ≥99.0%, or ≥99.5%.

In another embodiment of the second aspect of the invention, the 1-ethyl-N-((1,2,3,5,6,7-hexahydro-s-indacen-4-yl)carbamoyl)piperidine-4-sulfonamide or the salt thereof has a HPLC purity of ≥95.0%. More typically, the 1-ethyl-N-((1,2,3,5,6,7-hexahydro-s-indacen-4-yl)carbamoyl)piperidine-4-sulfonamide or the salt thereof has a HPLC purity of ≥98.0%, or ≥99.0%, or ≥99.5%, or ≥99.8%, or ≥99.9%.

In one embodiment of the first aspect of the invention, the 1,2,3,5,6,7-hexahydro-s-indacene derivative (B) or (B′) is prepared by a process according to the third aspect of the invention.

In one embodiment of the first aspect of the invention, the 1-ethyl-4-piperidine-sulfonamide (A) is prepared by a process according to the fifth aspect of the invention.

A third aspect of the invention provides a process of preparing a 1,2,3,5,6,7-hexahydro-s-indacene derivative (B) or a salt thereof, the process comprising the step of converting 1,2,3,5,6,7-hexahydro-s-indacen-4-amine (D) into the 1,2,3,5,6,7-hexahydro-s-indacene derivative (B) or the salt thereof:

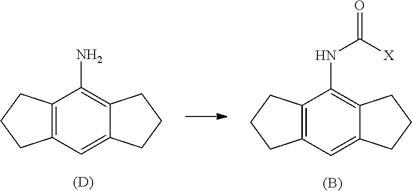

wherein X is a leaving group.

In the third aspect of the invention, X may be as defined in accordance with any embodiment of the first aspect of the invention.

In one embodiment of the third aspect of the invention, the process comprises the step of contacting 1,2,3,5,6,7-hexahydro-s-indacen-4-amine (D) with reagent (E):

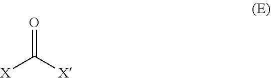

optionally in the presence of a base and/or a solvent, wherein X is as defined above and X′ is a leaving group.

In one embodiment of the third aspect of the invention, X′ is Cl, Br, I, OR¹, SR¹, N(R¹)₂, OP(═O)(R¹)₂ or OP(R¹)₃⁺, wherein each R¹ is as defined in accordance with the first aspect of the invention. Typically, X′ is Cl, Br or I. More typically, X′ is Cl or Br. Most typically, X′ is Cl.

X and X′ may be the same or different. Typically X and X′ are different. Typically X and X′ are selected such that X′ is more readily displaced than X.

In one embodiment of the third aspect of the invention, X′ is Cl, Br or I, and X is OR¹, SR¹, N(R¹)₂, OP(═O)(R¹)₂ or OP(R¹)₃⁺. More typically, X′ is Cl or Br, and X is OR¹, SR¹ or N(R¹)₂.

In one embodiment of the third aspect of the invention, X′ is Cl, Br or I, and X is OR¹, wherein R¹ is selected from an alkyl, cycloalkyl, aryl, heteroaryl, arylalkyl or heteroarylalkyl group, wherein R¹ may optionally be substituted with one or more substituents independently selected from halo, —CN, —OH, —NO₂, —NH₂, oxo (═O), ═NH, —R¹⁰, —OR¹⁰, —NHR¹⁰, —N(R¹⁰)₂, —N(O)(R¹⁰)₂, or ═NR¹⁰, wherein each R¹⁰ is independently selected from a C₁-C₄ alkyl, C₁-C₄ haloalkyl, C₃-C₄ cycloalkyl or C₃-C₄ halocycloalkyl group, or any two R¹⁰ directly attached to the same nitrogen atom may together form a C₂-C₅ alkylene or C₂-C₅ haloalkylene group, and wherein R¹, including any optional substituents, contains from 1 to 20 carbon atoms.

More typically, X′ is Cl or Br, and X is OR¹, wherein R¹ is selected from an aryl or heteroaryl group, wherein the aryl or heteroaryl group is monocyclic, bicyclic or tricyclic, wherein R¹ may optionally be substituted with one or more substituents independently selected from halo, —CN, —OH, —NO₂, —NH₂, —R¹⁰, —OR¹⁰, —NHR¹⁰, —N(R¹⁰)₂ or —N(O)(R¹⁰)₂, wherein each R¹⁰ is independently selected from a C₁-C₄ alkyl, C₁-C₄ haloalkyl, C₃-C₄ cycloalkyl or C₃-C₄ halocycloalkyl group, or any two R¹⁰ directly attached to the same nitrogen atom may together form a C₂-C₅ alkylene or C₂-C₅ haloalkylene group, and wherein R¹, including any optional substituents, contains from 1 to 20 carbon atoms.

More typically still, X′ is Cl and X is OR¹, wherein R¹ is a phenyl group, wherein the phenyl group is optionally substituted with one or more fluoro, chloro or —NO₂ groups. Most typically, X′ is Cl and X is OPh.

Accordingly, in one embodiment of the third aspect of the invention, there is provided a process of preparing 4-(phenoxycarbonylamino)-1,2,3,5,6,7-hexahydro-s-indacene (B′), the process comprising the step of contacting 1,2,3,5,6,7-hexahydro-s-indacen-4-amine (D) with phenyl chloroformate (E′), optionally in the presence of a solvent and/or a base:

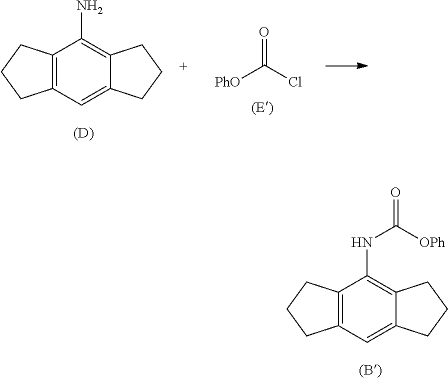

Typically, 1,2,3,5,6,7-hexahydro-s-indacen-4-amine (D) is contacted with reagent (E) or (E′) in the presence of a solvent. In one embodiment, the solvent is a polar aprotic solvent such as dimethyl sulfoxide, N,N-dimethylformamide, N,N′-dimethylpropyleneurea, tetrahydrofuran, 1,4-dioxane, ethyl acetate, acetone, acetonitrile, dichloromethane, hexamethylphosphoramide, nitromethane, propylene carbonate, N-methyl pyrrolidone, or a mixture thereof. Typically the solvent does not comprise an ester. More typically the solvent does not comprise a carbonyl group. Typically the solvent is not halogenated. For example, the solvent may be selected from dimethyl sulfoxide, tetrahydrofuran, 1,4-dioxane, acetonitrile, hexamethylphosphoramide, nitromethane, or a mixture thereof. More typically still, the solvent does not comprise a carbonyl, C═N or C≡N group. Typically, where the solvent does not comprise a carbonyl, C═N or C≡N group, the solvent is not halogenated. For example, the solvent may be selected from dimethyl sulfoxide, tetrahydrofuran, 1,4-dioxane, hexamethylphosphoramide, nitromethane, or a mixture thereof. Most typically, the solvent is tetrahydrofuran.

Typically, 1,2,3,5,6,7-hexahydro-s-indacen-4-amine (D) is contacted with reagent (E) or (E′) in the presence of a base. Typically, the base is a sterically hindered base. For example, the base may be a tertiary amine such as N,N-diisopropylethylamine (DIPEA), trimethylamine, triethylamine (TEA), tripropylamine or tributylamine. Most typically, the base is N,N-diisopropylethylamine.

Typically in accordance with the third aspect of the invention, the 1,2,3,5,6,7-hexahydro-s-indacene derivative (B) or (B′) is prepared in non-salt form.

In one embodiment of the third aspect of the invention, 1,2,3,5,6,7-hexahydro-s-indacen-4-amine (D) is combined with reagent (E) or (E′) at a temperature in the range from −10 to 40° C. Typically, 1,2,3,5,6,7-hexahydro-s-indacen-4-amine (D) is combined with reagent (E) or (E′) at a temperature in the range from 0 to 25° C., more typically in the range from 0 to 10° C.

In one embodiment of the third aspect of the invention, after the 1,2,3,5,6,7-hexahydro-s-indacen-4-amine (D) has been combined with reagent (E) or (E′), the reaction mixture is allowed to warm to a temperature in the range from 5 to 50° C. Typically, the reaction mixture is allowed to warm to a temperature in the range from 10 to 30° C., more typically in the range from 15 to 25° C.

Typically in accordance with the third aspect of the invention the 1,2,3,5,6,7-hexahydro-s-indacen-4-amine (D) is present in or added to the solvent at an initial concentration of from 0.01 to 10 mol/L relative to the total volume of solvent used in the reaction mixture. More typically, the 1,2,3,5,6,7-hexahydro-s-indacen-4-amine (D) is present in or added to the solvent at an initial concentration of from 0.1 to 1.0 mol/L. Most typically the 1,2,3,5,6,7-hexahydro-s-indacen-4-amine (D) is present in or added to the solvent at an initial concentration of from 0.4 to 0.5 mol/L.

Typically, the process of the third aspect of the invention uses from 0.9 to 1.5 molar equivalents of reagent (E) or (E′), relative to the initial amount 1,2,3,5,6,7-hexahydro-s-indacen-4-amine (D). More typically, the process uses from 1.0 to 1.2 molar equivalents of the reagent (E) or (E′). Most typically, the process uses from 1.05 to 1.15 molar equivalents of reagent (E) or (E′).

Typically, the process of the third aspect of the invention uses from 0.8 to 2.0 molar equivalents of the base, relative to the initial amount 1,2,3,5,6,7-hexahydro-s-indacen-4-amine (D). More typically, the process uses from 1.0 to 1.5 molar equivalents of the base. Most typically, the process uses from 1.1 to 1.3 molar equivalents of the base.

In one embodiment of the third aspect of the invention, the process comprises the steps of:
(i) dissolving the 1,2,3,5,6,7-hexahydro-s-indacen-4-amine (D) in a first portion of the solvent;
(ii) dissolving the base in a second portion of the solvent and adding the resultant solution to the solution formed in step (i); and
(iii) dissolving reagent (E) or (E′) in a third portion of the solvent and adding the resultant solution to the mixture formed in step (ii).

In one embodiment of the third aspect of the invention, at the end of the reaction the process further comprises the steps of:
(i) concentrating the reaction mixture under vacuum; then
(ii) optionally adding a co-solvent and concentrating the resultant mixture under vacuum.

Step (ii) may be repeated one or more times. Typically the co-solvent is an alcohol such as methanol or ethanol. Most typically the co-solvent is ethanol.

In one embodiment of the third aspect of the invention, the 1,2,3,5,6,7-hexahydro-s-indacene derivative (B) or the salt thereof is purified and/or isolated by crystallisation or precipitation. For example, a precipitation solvent may be added to the concentrated reaction mixture to create a precipitation mixture from which the 1,2,3,5,6,7-hexahydro-s-indacene derivative (B) or the salt thereof is precipitated, optionally under cooling. Typically the precipitation solvent is an alcohol such as methanol or ethanol. Most typically the precipitation solvent is ethanol.

Typically, a non-salt form of the 1,2,3,5,6,7-hexahydro-s-indacene derivative (B) is isolated by crystallisation or precipitation. Most typically, a non-salt form of 4-(phenoxycarbonylamino)-1,2,3,5,6,7-hexahydro-s-indacene (B′) is isolated by crystallisation or precipitation.

Typically the crystallisation or precipitation occurs at a temperature in the range from −10 to 20° C. More typically, the crystallisation or precipitation occurs at a temperature in the range from −5 to 10° C., and most typically in the range from 0 to 5° C.

A fourth aspect of the invention provides a 1,2,3,5,6,7-hexahydro-s-indacene derivative (B) or a salt thereof:

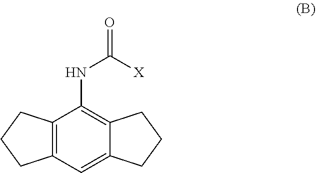

wherein X is a leaving group.

In the fourth aspect of the invention, X may be as defined in accordance with any embodiment of the first aspect of the invention.

A particular embodiment of the fourth aspect of the invention provides 4-(phenoxycarbonylamino)-1,2,3,5,6,7-hexahydro-s-indacene (B′) or a salt thereof:

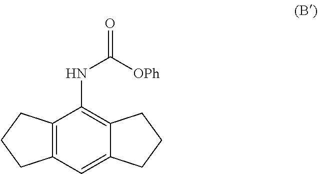 [SMILES: O=C(Nc1c2c(cc3c1CCC3)CCC2)Oc1ccccc1]

The 1,2,3,5,6,7-hexahydro-s-indacene derivative (B) or the salt thereof, or the 4-(phenoxycarbonylamino)-1,2,3,5,6,7-hexahydro-s-indacene (B′) or the salt thereof, may be prepared by or preparable by a process of the third aspect of this invention.

Typically the 1,2,3,5,6,7-hexahydro-s-indacene derivative (B) or the 4-(phenoxy-carbonylamino)-1,2,3,5,6,7-hexahydro-s-indacene (B′) of the fourth aspect of the invention is in non-salt form.

In one embodiment of the fourth aspect of the invention, the 1,2,3,5,6,7-hexahydro-s-indacene derivative (B) or the salt thereof has a HPLC purity of ≥96.0%. More typically, the 1,2,3,5,6,7-hexahydro-s-indacene derivative (B) or the salt thereof has a HPLC purity of ≥98.0%, or ≥99.0%, or ≥99.5%, or ≥99.6%.

In another embodiment of the fourth aspect of the invention, the 4-(phenoxy-carbonylamino)-1,2,3,5,6,7-hexahydro-s-indacene (B′) or the salt thereof has a HPLC purity of ≥96.0%. More typically, the 4-(phenoxycarbonylamino)-1,2,3,5,6,7-hexahydro-s-indacene (B′) or the salt thereof has a HPLC purity of ≥98.0%, ≥99.0%, or ≥99.5% or ≥99.6%.

In one embodiment of the third aspect of the invention, the 1,2,3,5,6,7-hexahydro-s-indacen-4-amine (D) is prepared by a process comprising one or more steps selected from:
(i) contacting 2,3-dihydro-1H-indene (L) with YCH₂CH₂C(O)Z (M) to obtain a substituted 1-(2,3-dihydro-1H-inden-5-yl)propan-1-one (N), wherein Y and Z are leaving groups:

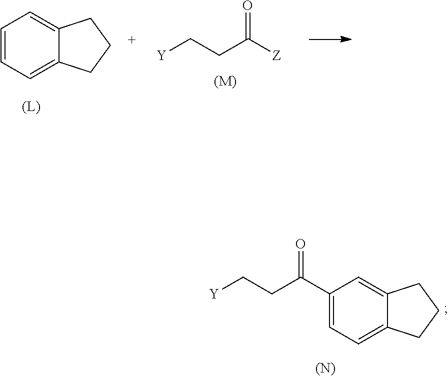

(ii) contacting the substituted 1-(2,3-dihydro-1H-inden-5-yl)propan-1-one (N) with an acid to obtain 1,2,3,5,6,7-hexahydro-s-indacen-1-one (P):

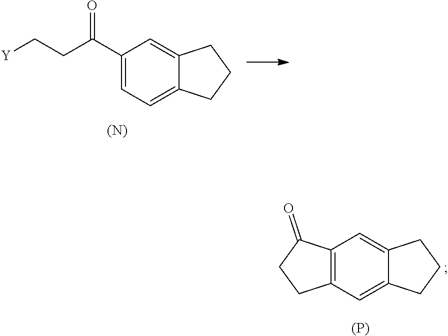

(iii) converting 1,2,3,5,6,7-hexahydro-s-indacen-1-one (P) into 8-nitro-1,2,3,5,6,7-hexahydro-s-indacen-1-one (Qa) and/or 4-nitro-1,2,3,5,6,7-hexahydro-s-_indacen-1-one (Qb):

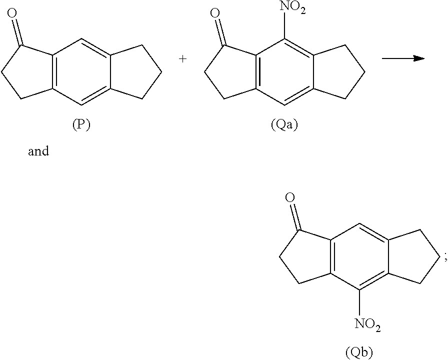

(iv) reducing 8-nitro-1,2,3,5,6,7-hexahydro-s-indacen-1-one (Qa) and/or 4-nitro-1,2,3,5,6,7-hexahydro-s-indacen-1-one (Qb) to obtain 1,2,3,5,6,7-hexahydro-s-indacen-4-amine (D):

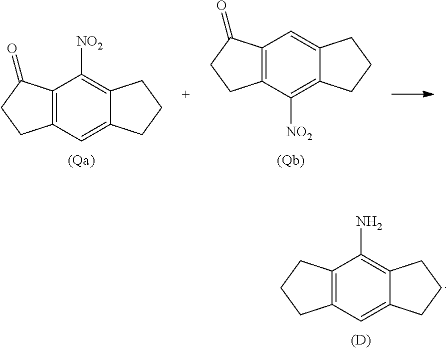

In one embodiment, the process comprises one, two, three or all four of steps (i) to (v).

The process for the preparation of 1,2,3,5,6,7-hexahydro-s-indacen-4-amine may be as described in WO 2020/079207 A1, the contents of which are incorporated herein by reference in their entirety.

In one embodiment, in step (i), the leaving group Y is independently selected from Cl, Br, I, or a sulphonate leaving group such as a toluenesulfonate, methanesulfonate, or trifluoromethanesulfonate leaving group.

In one embodiment, in step (i), the leaving group Z is independently selected from Cl, Br, I, OR¹, SR¹, N(R¹)₂, OP(═O)(R¹)₂ or OP(R¹)₃⁺, wherein R¹ is as defined in relation to the first embodiment of the invention.

Y and Z may be the same or different. Typically, Y and Z are each independently selected from Cl, Br and I. Typically, at least one of Y and Z is Cl. More typically, Y and Z are both Cl. When both Y and Z are Cl, in step (i) 2,3-dihydro-1H-indene (L) is contacted with 3-chloropropionyl chloride to obtain 3-chloro-1-(2,3-dihydro-1H-inden-5-yl)propan-1-one.

In one embodiment, the reaction of step (i) is carried out in the presence of a catalyst, such as a Lewis acid such as aluminium chloride.

Step (i) may be carried out in the presence of a solvent. In one embodiment, the solvent is an aprotic solvent. In one embodiment, the solvent is dichloromethane, dichloroethane, chloroform, diethyl ether, n-pentane, n-hexane, n-heptane, toluene, or a mixture thereof. Typically, the solvent is dichloromethane.

In one embodiment, the reaction of step (i) is carried out at a temperature in the range from −20 to 50° C. Typically, the reaction of step (i) is carried out at a temperature in the range from −15 to 25° C., more typically at a temperature in the range from −10 to 15° C.

In one embodiment, in step (ii), the acid is sulfuric acid, hydrochloric acid, Eaton's reagent, polyphosphoric acid or a mixture thereof. Typically, the acid is sulfuric acid or hydrochloric acid. More typically, the acid is sulfuric acid. Typically, no additional solvent is used.

In one embodiment, the reaction of step (ii) is carried out at a temperature in the range from 10 to 90° C. Typically, the reaction of step (ii) is carried out at a temperature in the range from 40 to 80° C., more typically at a temperature in the range from 65 to 70° C.

In one embodiment, in step (iii), 1,2,3,5,6,7-hexahydro-s-indacen-1-one (P) is converted into 8-nitro-1,2,3,5,6,7-hexahydro-s-indacen-1-one (Qa) or 4-nitro-1,2,3,5,6,7-hexahydro-s-indacen-1-one (Qb) or a mixture thereof by treatment with sulfuric acid and nitric acid. Typically, no additional solvent is used.

In one embodiment, the reaction of step (iii) is carried out at a temperature in the range from 0 to 20° C. Typically, the reaction of step (iii) is carried out at a temperature in the range from 0 to 10° C., more typically at a temperature in the range from 0 to 5° C.

In one embodiment, the reactions of steps (ii) and (iii) are carried out without isolating 1,2,3,5,6,7-hexahydro-s-indacen-1-one (P).

In one embodiment, the reduction of step (iv) is carried out using a catalyst and hydrogen gas. Typically, the catalyst is a metal catalyst comprising platinum, palladium, rhodium, ruthenium or nickel. Typically, the catalyst is Pd/C, Pd(OH)₂/C, Pt/C, PtO₂, platinum black or Raney nickel. More typically, the catalyst is Pd/C or Pd(OH)₂/C. Most typically, the catalyst is Pd(OH)₂/C. Typically, the hydrogen gas is provided at a pressure of 80-120 Psi, typically about 100 Psi. The catalyst and hydrogen gas may be used in the presence of an acid such as sulfuric acid or a sulfonic acid such as methanesulfonic acid or p-toluenesulfonic acid (PTSA). Most typically, Pd(OH)₂/C and hydrogen gas are used in the presence of methanesulfonic acid.

In one embodiment, the reduction of step (iv) is carried out in the presence of a solvent. Typically, the solvent is a polar solvent such as methanol, ethanol, ethyl acetate, isopropanol, n-butanol, THF, water, acetic acid or a mixture thereof. Typically, the solvent is a polar protic solvent. More typically the solvent is an alcohol such as methanol, ethanol, isopropanol or n-butanol. Most typically, the solvent is methanol.

In one embodiment, the reduction of step (iv) is carried out at a temperature in the range from 10 to 80° C. Typically, the reduction of step (iv) is carried out at a temperature in the range from 20 to 60° C.

In one specific embodiment of the third aspect of the invention, there is provided a process of preparing 4-(phenoxycarbonylamino)-1,2,3,5,6,7-hexahydro-s-indacene (B′) or a salt thereof:

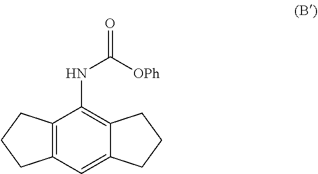 [SMILES: O=C(Nc1c2c(cc3c1CCC3)CCC2)Oc1ccccc1]

comprising the steps of:
(i) contacting 2,3-dihydro-1H-indene (L) with 3-chloropropionyl chloride (M′) in the presence of a Lewis acid to obtain 3-chloro-1-(2,3-dihydro-1H-inden-5-yl)propan-1-one (N′):

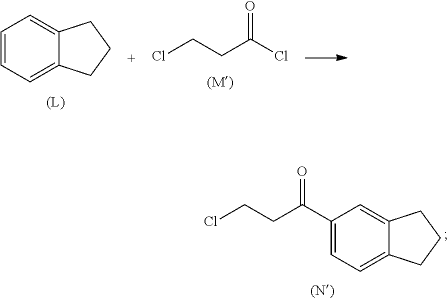

(ii) contacting 3-chloro-1-(2,3-dihydro-1H-inden-5-yl)propan-1-one (N′) with an acid to obtain 1,2,3,5,6,7-hexahydro-s-indacen-1-one (P):

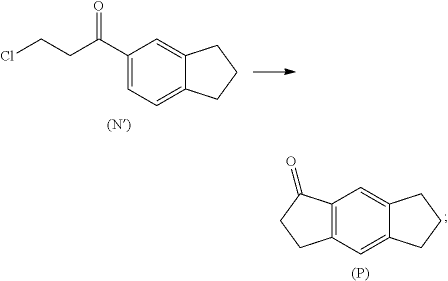

(iii) converting 1,2,3,5,6,7-hexahydro-s-indacen-1-one (P) into 8-nitro-1,2,3,5,6,7-hexahydro-s-indacen-1-one (Qa) and/or 4-nitro-1,2,3,5,6,7-hexahydro-s-indacen-1-one (Qb) by treatment with sulfuric acid and nitric acid:

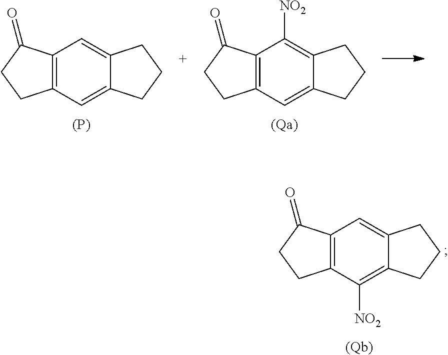

(iv) reducing 8-nitro-1,2,3,5,6,7-hexahydro-s-indacen-1-one (Qa) and/or 4-nitro-1,2,3,5,6,7-hexahydro-s-indacen-1-one (Qb) to obtain 1,2,3,5,6,7-hexahydro-s-indacen-4-amine (D):

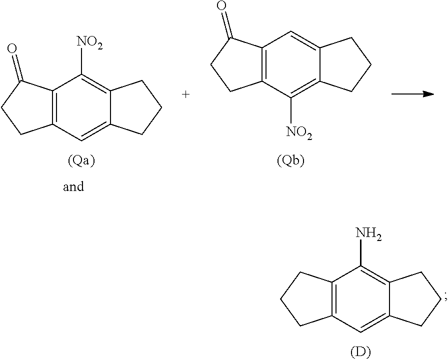

v) converting 1,2,3,5,6,7-hexahydro-s-indacen-4-amine (D) into 4-(phenoxycarbonylamino)-1,2,3,5,6,7-hexahydro-s-indacene (B′) by contacting 1,2,3,5,6,7-hexahydro-s-indacen-4-amine with PhOC(O)L:

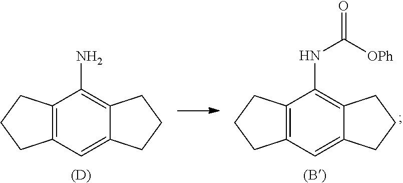

wherein L is selected from Cl and Br.

A fifth aspect of the invention provides a process comprising one or more steps selected from:
(a) converting 4-hydroxy piperidine (F) to a N-protected-4-hydroxy piperidine (G):

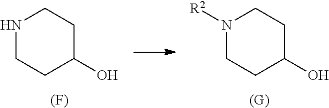

wherein R² is a nitrogen protecting group;
(b) converting a N-protected-4-hydroxy piperidine (G) to a N-protected-4-derivatised piperidine (H):

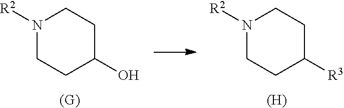

wherein R² is a nitrogen protecting group and R³ is a leaving group;
(c) converting a N-protected-4-derivatised piperidine (H) to a N-protected-4-(acylthio)-piperidine (I):

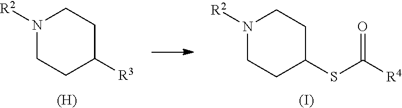

wherein R² is a nitrogen protecting group, R³ is a leaving group, and R⁴ is a C₁-C₂₀ hydrocarbyl group, wherein the C₁-C₂₀ hydrocarbyl group may be straight-chained or branched, or be or include one or more cyclic groups, wherein the C₁-C₂₀ hydrocarbyl group may optionally be substituted with one or more oxo (═O) and/or one or more halo groups, and wherein the C₁-C₂₀ hydrocarbyl group may optionally include one or more heteroatoms independently selected from N, O and S in its carbon skeleton;
(d) converting a N-protected-4-(acylthio)-piperidine (I) to a N-protected-4-(halosulfonyl)-piperidine (J):

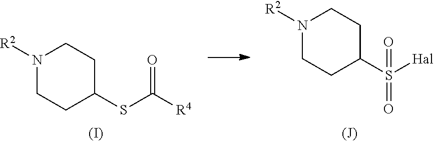

wherein R² is a nitrogen protecting group, R⁴ is a C₁-C₂₀ hydrocarbyl group, wherein the C₁-C₂₀ hydrocarbyl group may be straight-chained or branched, or be or include one or more cyclic groups, wherein the C₁-C₂₀ hydrocarbyl group may optionally be substituted with one or more oxo (═O) and/or one or more halo groups, and wherein the C₁-C₂₀ hydrocarbyl group may optionally include one or more heteroatoms independently selected from N, O and S in its carbon skeleton, and Hal is Cl or Br;
(e) converting a N-protected-4-(halosulfonyl)-piperidine (J) to a N-protected-4-piperidinesulfonamide (K):

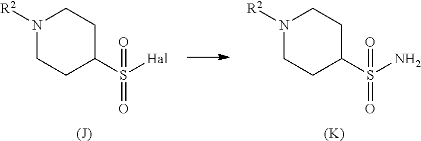

wherein R² is a nitrogen protecting group and Hal is Cl or Br; and
(f) converting a N-protected-4-piperidinesulfonamide (K) to 1-ethyl-4-piperidinesulfonamide (A):

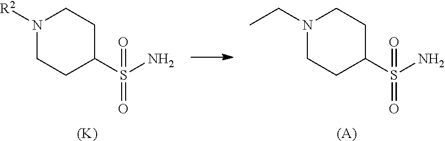

wherein R² is a nitrogen protecting group.

In one embodiment of the fifth aspect of the invention, the process comprises one, two, three, four, five or all six of steps (a) to (f).

In one embodiment, the process of the fifth aspect of the invention is a process of preparing 1-ethyl-4-piperidinesulfonamide (A) or a salt thereof:

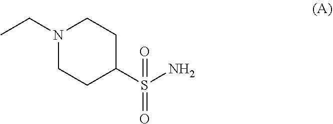

Typically, where the process of the fifth aspect of the invention is a process for preparing 1-ethyl-4-piperidinesulfonamide (A) or a salt thereof, the process comprises at least step (f). In one embodiment, the process comprises steps (e) and (f). In another embodiment, the process comprises steps (d), (e) and (f). In another embodiment, the process comprises steps (c), (d), (e) and (f). In another embodiment, the process comprises steps (b), (c), (d), (e) and (f). In another embodiment, the process comprises all six of steps (a), (b), (c), (d), (e) and (f).

As will be understood, where the process of the fifth aspect of the invention comprises two or more consecutive steps selected from steps (a) to (f), in each consecutive step R² is the same. Similarly, where the process of the fifth aspect of the invention comprises steps (b) and (c), in each step R³ is the same. Likewise, where the process of the fifth aspect of the invention comprises steps (c) and (d), in each step R⁴ is the same.

As stated, R² is a nitrogen protecting group. Suitable nitrogen protecting groups may be identified by reference to e.g. Wuts, ‘Greene's Protective Groups in Organic Synthesis’, 5^{th} Ed., 2014, the contents of which are incorporated herein by reference in their entirety.

In one embodiment of the fifth aspect of the invention, R² is a nitrogen protecting group that is stable under basic conditions. Typically, R² is also stable under weak nucleophilic conditions, such as on exposure to MeCOS⁻. For example, R² may be selected from the group consisting of benzyloxycarbonyl (CBz), 4-methoxy-benzyloxycarbonyl, benzyl, t-butoxycarbonyl (Boc), 2-(4-biphenylyl)-isopropoxycarbonyl (Bpoc), triphenylmethyl (Trt) and 2,2,2-trichloroethoxycarbonyl (Troc) protecting groups.

In one embodiment of the fifth aspect of the invention, R² is a nitrogen protecting group that may be removed by catalytic hydrogenolysis. Typically, R² is a nitrogen protecting group that is stable under basic conditions, and that may be removed by catalytic hydrogenolysis. More typically, R² is a nitrogen protecting group that is stable under basic and weak nucleophilic conditions, and that may be removed by catalytic hydrogenolysis. For example, R² may be selected from the group consisting of benzyloxycarbonyl (CBz), 4-methoxy-benzyloxycarbonyl, benzyl, 2-(4-biphenylyl)-isopropoxycarbonyl (Bpoc) or triphenylmethyl (Trt) group.

In a further embodiment of the fifth aspect of the invention, R² is —CH₂R²⁰ or —COOCH₂R²⁰, wherein R²⁰ is an aryl or heteroaryl group, wherein the aryl or heteroaryl group is monocyclic, bicyclic or tricyclic, wherein the aryl or heteroaryl group may optionally be substituted with one or more substituents independently selected from halo, —CN, —OH, —NO₂, —NH₂, —R²¹, —OR²¹, —NHR²¹, —N(R²¹)₂ or —N(O)(R²¹)₂, wherein each R²¹ is independently selected from a C₁-C₄ alkyl, C₁-C₄ haloalkyl, C₃-C₄ cycloalkyl or C₃-C₄ halocycloalkyl group, or any two R²¹ directly attached to the same nitrogen atom may together form a C₂-C₅ alkylene or C₂-C₅ haloalkylene group, and wherein R²⁰, including any optional substituents, contains from 1 to 20 carbon atoms.

In one embodiment of the fifth aspect of the invention, R² is —COOCH₂R²⁰.

In one embodiment of the fifth aspect of the invention, R²⁰ is selected from a phenyl or a monocyclic heteroaryl group, wherein R²⁰ may optionally be substituted with one or more substituents independently selected from halo, —CN, —OH, —NO₂, —NH₂, -Me, -Et, —OMe, —OEt, —NHMe, —NHEt, —N(Me)₂, —N(Me)Et or —N(Et)₂, wherein any methyl (Me) or ethyl (Et) group may optionally be substituted with one or more halo groups, and wherein R²⁰, including any optional substituents, contains from 1 to 12 carbon atoms.

Typically, R²⁰ is a phenyl group, wherein the phenyl group is optionally substituted with one or more fluoro, chloro, —OMe, —OEt, or —NO₂ groups.

More typically, R²⁰ is a phenyl group. For example, R² may be —CH₂Ph or —COOCH₂Ph.

Most typically, R² is —COOCH₂Ph (i.e. a benzyloxycarbonyl (CBz) group).

As stated, R³ is a leaving group. In one embodiment of the fifth aspect of the invention, R³ is selected from Cl, Br, I, or a sulphonate leaving group such as a toluenesulfonate (tosylate or —OTs), methanesulfonate (mesylate or —OMs), or trifluoromethanesulfonate (triflate or —OTf) leaving group. Typically, R³ is a sulphonate leaving group. Most typically R³ is —OMs.

In one embodiment of the fifth aspect of the invention, R⁴ is selected from an alkyl, cycloalkyl, aryl, heteroaryl, arylalkyl or heteroarylalkyl group, wherein R⁴ may optionally be substituted with one or more substituents independently selected from halo, —CN, —OH, —NO₂, —NH₂, oxo (═O), -Me, -Et, —OMe, —OEt, —NHMe, —NHEt, —N(Me)₂, —N(Me)Et or —N(Et)₂, wherein any methyl (Me) or ethyl (Et) group may optionally be substituted with one or more halo groups, and wherein R⁴, including any optional substituents, contains from 1 to 12 carbon atoms. More typically, R⁴ is a C₁-C₆ alkyl or C₁-C₆ haloalkyl group, such as a methyl, trifluoromethyl, ethyl or isopropyl group. Most typically, R⁴ is methyl.

As stated, Hal is Cl or Br. Typically, Hal is Cl.

In one embodiment of the fifth aspect of the invention, the reaction step (a) comprises contacting 4-hydroxy piperidine (F) with a nitrogen protecting group precursor. In one embodiment, the nitrogen protecting group precursor is X²—R², wherein X² is a leaving group. For example, X²—R² may be X²—CH₂R²⁰, wherein R²⁰ is as defined above and X² is selected from Cl, Br, I, or a sulphonate leaving group such as a toluenesulfonate, methanesulfonate, or trifluoromethanesulfonate leaving group. Typically in such an embodiment, X² is selected from Cl or Br. In one aspect of such an embodiment, X²—R² is Br—CH₂R²⁰, such as Br—CH₂Ph. Alternately, X²—R² may be X²—COOCH₂R²⁰, wherein R²⁰ is as defined above and X² is selected from Cl, Br, I, OR¹, SR¹, N(R¹)₂, OP(═O)(R¹)₂ or OP(R¹)₃⁺, wherein R¹ is as defined in relation to the first embodiment of the invention. Typically where X²—R² is X²—COOCH₂R²⁰, X² is selected from Cl, Br or I. More typically in such an embodiment, X²—R² is Cl—COOCH₂R²⁰, most typically Cl—COOCH₂Ph.

Typically, the reaction step (a) is carried out in the presence of a solvent. Typically, the solvent is a polar solvent or a mixture of polar and non-polar solvents. For example, the solvent may comprise one or more polar protic solvents and/or one or more polar aprotic solvents and/or one or more non-polar solvents. Suitable polar protic solvents include water and alcohols such as methanol, ethanol, isopropanol or n-butanol. Suitable polar aprotic solvents include dimethyl sulfoxide, N,N-dimethylformamide, N,N′-dimethylpropyleneurea, tetrahydrofuran, 1,4-dioxane, ethyl acetate, acetone, acetonitrile, dichloromethane, hexamethylphosphoramide, nitromethane, propylene carbonate and N-methyl pyrrolidone. Suitable non-polar solvents include pentane, cyclopentane, hexane, cyclohexane, diethyl ether and toluene.

In one embodiment, the reaction step (a) is carried out in the presence of a polar protic solvent such as water, a polar aprotic solvent such as 1,4-dioxane, and a non-polar solvent such as toluene. Typically, in such an embodiment, the solvent mixture comprises from 30 to 50 vol. % of the polar protic solvent, from 30 to 50 vol. % of the polar aprotic solvent, and from 10 to 30 vol. % of the non-polar solvent.

Typically, the reaction step (a) comprises contacting the 4-hydroxy piperidine (F) with the nitrogen protecting group precursor (e.g. X²—R² or Cl—COOCH₂Ph) in the presence of a base. In one embodiment, the base is selected from a carbonate, hydrogen carbonate, hydroxide or alkoxide base. Typically the base is a hydroxide or alkoxide base such as an alkali metal hydroxide, an alkali earth metal hydroxide, an alkali metal alkoxide, or an alkali earth metal alkoxide. More typically the base is a hydroxide such as an alkali metal hydroxide or an alkali earth metal hydroxide. More typically still, the base is an alkali metal hydroxide such as lithium hydroxide, potassium hydroxide or sodium hydroxide. Most typically, the base is sodium hydroxide.

In an exemplary embodiment of the fifth aspect of the invention, the reaction step (a) comprises contacting 4-hydroxy piperidine (F) with benzyl chloroformate to obtain N-carboxybenzyl-4-hydroxy piperidine (G′):

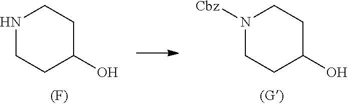

Typically in such an embodiment, the 4-hydroxy piperidine (F) is contacted with the benzyl chloroformate in the presence of sodium hydroxide and a solvent.

In one embodiment of the fifth aspect of the invention, the reaction step (a) is carried out at a temperature in the range from 0 to 60° C. Typically, the reaction of step (a) is carried out at a temperature in the range from 10 to 50° C. More typically, the reaction of step (a) is carried out at a temperature in the range from 20 to 40° C.

Typically in accordance with the fifth aspect of the invention, in step (a) the 4-hydroxy piperidine (F) is present in or added to the solvent at an initial concentration of from 0.01 to 10 mol/L relative to the total volume of solvent used in the reaction mixture. More typically, the 4-hydroxy piperidine (F) is present in or added to the solvent at an initial concentration of from 0.5 to 1.0 mol/L. Most typically the 4-hydroxy piperidine (F) is present in or added to the solvent at an initial concentration of from 0.7 to 0.8 mol/L.

Typically, the process of step (a) of the fifth aspect of the invention uses from 0.5 to 2.0 molar equivalents of the nitrogen protecting group precursor (e.g. X²—R² or Cl—COOCH₂Ph), relative to the initial amount of 4-hydroxy piperidine (F). More typically, the process uses from 0.8 to 1.1 molar equivalents of the nitrogen protecting group precursor. Most typically, the process uses from 0.9 to 1.0 molar equivalents of the nitrogen protecting group precursor.

Typically, the process of step (a) of the fifth aspect of the invention uses from 0.8 to 1.5 molar equivalents of the base, relative to the initial amount of 4-hydroxy piperidine (F). More typically, the process uses from 0.9 to 1.2 molar equivalents of the base. Most typically, the process uses from 1.0 to 1.1 molar equivalents of the base.

In one embodiment of the fifth aspect of the invention, the process of step (a) comprises the steps of:
(i) combining the 4-hydroxy piperidine (F) with a first portion of the solvent to form a first mixture;
(ii) dissolving the base in a second portion of the solvent and adding the resultant solution to the mixture formed in step (i) to form a second mixture; and
(iii) dissolving the nitrogen protecting group precursor in a third portion of the solvent and adding the resultant solution to the mixture formed in step (ii) to form a third mixture.

Typically, the first portion of the solvent is or comprises a polar aprotic solvent such as 1,4-dioxane. Typically, the second portion of the solvent is or comprises a polar protic solvent such as water. Typically, the third portion of the solvent is or comprises a non-polar solvent such as toluene.

In one embodiment of the fifth aspect of the invention, at the end of the reaction the process of step (a) further comprises the step of partitioning the reaction mixture between one or more aqueous and one or more organic phases, wherein the N-protected-4-hydroxy piperidine (G) or (G′) is extracted into the one or more organic phases. Typically the one or more organic phases comprise an ether such as MTBE.

Optionally, one or more organic phases comprising the N-protected-4-hydroxy piperidine (G) or (G′) are:
(i) washed with an aqueous salt solution, such as a NaCl solution, and/or
(ii) dried over a sulfate such as magnesium sulfate or sodium sulfate.

Typically, after the extraction and any washing or drying steps, part or all of the solvent of the organic phase comprising the N-protected-4-hydroxy piperidine (G) or (G′) is removed under vacuum.

A sixth aspect of the invention provides an N-protected-4-hydroxy piperidine (G) or a salt thereof:

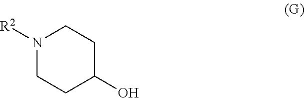

wherein R² is a nitrogen protecting group.

In the sixth aspect of the invention, R² may be as defined in accordance with any embodiment of the fifth aspect of the invention.

A particular embodiment of the sixth aspect of the invention provides N-carboxybenzyl-4-hydroxy piperidine (G′) or a salt thereof:

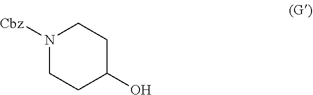 [SMILES: O=C(OCc1ccccc1)N1CCC(O)CC1]

The N-protected-4-hydroxy piperidine (G) or the salt thereof, or the N-carboxybenzyl-4-hydroxy piperidine (G′) or the salt thereof, may be prepared by or preparable by a process of step (a) of the fifth aspect of the invention.

Typically the N-protected-4-hydroxy piperidine (G) or the N-carboxybenzyl-4-hydroxy piperidine (G′) of the sixth aspect of the invention is in non-salt form.

In one embodiment of the fifth aspect of the invention, the reaction step (b) comprises contacting an N-protected-4-hydroxy piperidine (G), such as N-carboxybenzyl-4-hydroxy piperidine (G′), with a sulfonyl halide or a sulfonyl anyhdride to form N-protected-4-derivatised piperidine (H), wherein R³ is a sulfonate leaving group.

As will be appreciated, the sulfonyl halide or sulfonyl anyhydride used will correspond to the sulfonate leaving group of R³. For example, where R³ is a tosylate leaving group a tosyl halide or tosyl anhydride will be used. Similarly, where R³ is a mesylate leaving group a mesyl halide or mesyl anhydride will be used, and where R³ is a triflate leaving group a triflic halide or triflic anhydride will be used.

Typically, a sulfonyl halide is used. In one embodiment, the sulfonyl halide is selected from a sulfonyl chloride, a sulfonyl bromide, or a sulfonyl iodide. Typically, the sulfonyl halide is a sulfonyl chloride or a sulfonyl bromide. More typically, the sulfonyl halide is a sulfonyl chloride.

In a typical embodiment of the fifth aspect of the invention, the reaction step (b) comprises contacting an N-protected-4-hydroxy piperidine (G) with a mesyl halide or mesyl anyhdride to form N-protected-4-derivatised piperidine (H), wherein R³ is a mesylate leaving group. Most typically in such an embodiment the reaction step (b) comprises contacting an N-protected-4-hydroxy piperidine (G) with mesyl chloride.

Typically, the reaction step (b) is carried out in the presence of a solvent. In one embodiment, the solvent is a polar aprotic solvent such as dimethyl sulfoxide, N,N-dimethylformamide, N,N′-dimethylpropyleneurea, tetrahydrofuran, 1,4-dioxane, ethyl acetate, acetone, acetonitrile, dichloromethane, hexamethylphosphoramide, nitromethane, propylene carbonate, N-methyl pyrrolidone, or a mixture thereof.

Typically the solvent does not comprise an ester. More typically the solvent does not comprise a carbonyl group. For example, the solvent may be selected from dimethyl sulfoxide, tetrahydrofuran, 1,4-dioxane, acetonitrile, dichloromethane, hexamethylphosphoramide, nitromethane, or a mixture thereof. More typically still, the solvent does not comprise a carbonyl, C═N or C≡N group. For example, the solvent may be selected from dimethyl sulfoxide, tetrahydrofuran, 1,4-dioxane, dichloromethane, hexamethylphosphoramide, nitromethane, or a mixture thereof. Most typically, the solvent is dichloromethane.

In one embodiment of the fifth aspect of the invention, the reaction step (b) is carried out in the presence of a base. Typically, the base is a sterically hindered base. For example, the base may be a tertiary amine such as N,N-diisopropylethylamine (DIPEA), trimethylamine, triethylamine (TEA), tripropylamine or tributylamine. Most typically, the base is triethylamine (TEA).

In an exemplary embodiment of the fifth aspect of the invention, the reaction step (b) comprises contacting N-carboxybenzyl-4-hydroxy piperidine (G′) with mesyl chloride to obtain benzyl 4-((methylsulfonyl)oxy)piperidine-1-carboxylate (H′):

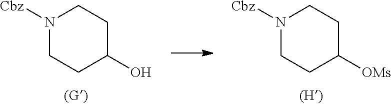 [SMILES: O=C(OCc1ccccc1)N1CCC(O)CC1 ; CS(=O)(=O)OC1CCN(C(=O)OCc2ccccc2)CC1]

Typically in such an embodiment, the N-carboxybenzyl-4-hydroxy piperidine (G′) is contacted with the mesyl chloride in the presence of a tertiary amine base such as triethylamine and a polar aprotic solvent such as dichloromethane.

In one embodiment of the fifth aspect of the invention, in step (b) the N-protected-4-hydroxy piperidine (G) or (G′) is combined with the sulfonyl halide or sulfonyl anyhydride at a temperature in the range from −20 to 20° C. Typically the N-protected-4-hydroxy piperidine (G) or (G′) is combined with the sulfonyl halide or sulfonyl anyhydride at a temperature in the range from −10 to 10° C., more typically in the range from −5 to 5° C.

In one embodiment of the fifth aspect of the invention, in step (b) after the N-protected-4-hydroxy piperidine (G) or (G′) has been combined with the sulfonyl halide or sulfonyl anyhydride, the reaction mixture is allowed to warm to a temperature in the range from 10 to 50° C. Typically, the reaction mixture is allowed to warm to a temperature in the range from 20 to 40° C., more typically to a temperature in the range from 25 to 30° C.

Typically in accordance with the fifth aspect of the invention, in step (b) the N-protected-4-hydroxy piperidine (G) or (G′) is present in or added to the solvent at an initial concentration of from 0.01 to 10 mol/L relative to the total volume of solvent used in the reaction mixture. More typically, the N-protected-4-hydroxy piperidine (G) or (G′) is present in or added to the solvent at an initial concentration of from 0.5 to 1.5 mol/L. Most typically the N-protected-4-hydroxy piperidine (G) or (G′) is present in or added to the solvent at an initial concentration of from 0.7 to 0.9 mol/L.

Typically, the process of step (b) of the fifth aspect of the invention uses from 0.9 to 2.0 molar equivalents of the sulfonyl halide or sulfonyl anyhydride, relative to the initial amount of the N-protected-4-hydroxy piperidine (G) or (G′). More typically, the process uses from 1.0 to 1.5 molar equivalents of the sulfonyl halide or sulfonyl anyhydride. Most typically, the process uses from 1.2 to 1.4 molar equivalents of the sulfonyl halide or sulfonyl anyhydride.

Typically, the process of step (b) of the fifth aspect of the invention uses from 1.0 to 3.0 molar equivalents of the base, relative to the initial amount of the N-protected-4-hydroxy piperidine (G) or (G′). More typically, the process uses from 1.5 to 2.5 molar equivalents of the base. Most typically, the process uses from 1.8 to 2.2 molar equivalents of the base.

In one embodiment of the fifth aspect of the invention, the process of step (b) comprises the steps of:
(i) combining the N-protected-4-hydroxy piperidine (G) or (G′) with a solvent to form a first mixture;
(ii) adding the base to the mixture formed in step (i) to form a second mixture; and
(iii) adding the sulfonyl halide or sulfonyl anyhydride to the mixture formed in step (ii) to form a third mixture.

In one embodiment of the fifth aspect of the invention, at the end of the reaction the process of step (b) further comprises the work-up steps of:
(i) optionally filtering off solids from the reaction mixture to provide a filtrate;
(ii) washing the reaction mixture or the filtrate with one or more aqueous washes, wherein the N-protected-4-derivatised piperidine (H) or (H′) is retained in the organic phase;
(iii) optionally drying the organic phase over a sulfate such as magnesium sulfate or sodium sulfate; and
(iv) optionally removing solvent from the organic phase under vacuum.

Typically the process of step (b) comprises all four of work-up steps (i) to (iv).

In one embodiment the one or more aqueous washes comprise washes with (i) aqueous sodium bicarbonate solution, (ii) water, and (iii) aqueous sodium chloride solution.

Optionally, the N-protected-4-derivatised piperidine (H) or (H′) is isolated by precipitation or crystallisation from a crystallisation solvent. Typically the crystallisation solvent comprises a mixture of polar aprotic and non-polar solvents, such as ethyl acetate and hexanes.

A seventh aspect of the invention provides an N-protected-4-derivatised piperidine (H) or a salt thereof:

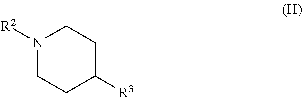

wherein R² is a nitrogen protecting group and R³ is a leaving group.

In the seventh aspect of the invention, R² and R³ may be as defined in accordance with any embodiment of the fifth aspect of the invention.

A particular embodiment of the seventh aspect of the invention provides benzyl 4-((methylsulfonyl)oxy)piperidine-1-carboxylate (H′) or a salt thereof:

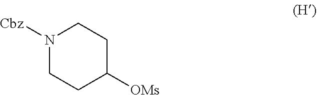 [SMILES: CS(=O)(=O)OC1CCN(C(=O)OCc2ccccc2)CC1]

The N-protected-4-derivatised piperidine (H) or the salt thereof, or the benzyl 4-((methylsulfonyl)oxy)piperidine-1-carboxylate (H′) or the salt thereof, may be prepared by or preparable by a process of step (b) of the fifth aspect of the invention.

Typically the N-protected-4-derivatised piperidine (H) or the benzyl 4-((methylsulfonyl)oxy)piperidine-1-carboxylate (H′) of the seventh aspect of the invention is in non-salt form.

In one embodiment of the seventh aspect of the invention, the N-protected-4-derivatised piperidine (H) or the salt thereof has a HPLC purity of ≥90%. More typically, the N-protected-4-derivatised piperidine (H) or the salt thereof has a HPLC purity of ≥94%.

In another embodiment of the seventh aspect of the invention, the benzyl 4-((methylsulfonyl)oxy)piperidine-1-carboxylate (H′) or the salt thereof has a HPLC purity of ≥90%. More typically, the benzyl 4-((methylsulfonyl)oxy)piperidine-1-carboxylate (H′) or the salt thereof has a HPLC purity of ≥94%.

In one embodiment of the fifth aspect of the invention, the reaction of step (c) comprises contacting the N-protected-4-derivatised piperidine (H) with R⁴COS⁻, wherein R⁴ is as defined above. Most typically, the reaction step (c) comprises contacting the N-protected-4-derivatised piperidine (H) with MeCOS⁻.

R⁴COS⁻ or MeCOS⁻ may be provided in salt form, or may be generated in situ by the reaction of the corresponding acid R⁴COSH or MeCOSH with a base. Typically, the R⁴COS⁻ or MeCOS⁻ is generated in situ. Where the R⁴COS⁻ or MeCOS⁻ is generated in situ, typically the R⁴COSH or MeCOSH is added to the reaction mixture after the addition of the base.

Where the R⁴COS⁻ or MeCOS⁻ is provided in salt form, typically the salt is an alkali metal salt, such as a sodium, potassium, rubidium or cesium salt, or an alkali earth metal salt such as a magnesium or calcium salt. More typically the salt is an alkali metal salt. Most typically, the salt is the cesium salt.

Where the R⁴COS⁻ or MeCOS⁻ is generated in situ, typically the base is a carbonate, hydrogen carbonate or hydroxide base, such as an alkali metal or alkali earth metal carbonate, an alkali metal hydrogen carbonate, or an alkali metal or alkali earth metal hydroxide. Typically the base is a carbonate. In one embodiment, the base is selected from cesium carbonate, cesium hydrogen carbonate or cesium hydroxide. Most typically, the base is cesium carbonate.

Typically, the reaction step (c) is carried out in the presence of a solvent. In one embodiment, the solvent is a polar aprotic solvent such as dimethyl sulfoxide, N,N-dimethylformamide, N,N′-dimethylpropyleneurea, tetrahydrofuran, 1,4-dioxane, ethyl acetate, acetone, acetonitrile, dichloromethane, hexamethylphosphoramide, nitromethane, propylene carbonate, N-methyl pyrrolidone, or a mixture thereof. Typically the solvent does not comprise an ester. Typically, the solvent is not halogenated. For example, the solvent may be selected from dimethyl sulfoxide, N,N-dimethylformamide, N,N′-dimethylpropyleneurea, tetrahydrofuran, 1,4-dioxane, acetone, acetonitrile, hexamethylphosphoramide, nitromethane, propylene carbonate, N-methyl pyrrolidone, or a mixture thereof. Most typically, the solvent is N,N-dimethylformamide.

In an exemplary embodiment of the fifth aspect of the invention, the reaction step (c) comprises contacting benzyl 4-((methylsulfonyl)oxy)piperidine-1-carboxylate (H′) with MeCOS⁻ in a solvent to obtain benzyl 4-(acetylthio)piperidine-1-carboxylate (I′):

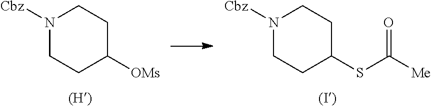 [SMILES: CS(=O)(=O)OC1CCN(C(=O)OCc2ccccc2)CC1 ; CC(=O)SC1CCN(C(=O)OCc2ccccc2)CC1]

Typically in such an embodiment, the MeCOS⁻ is generated in situ by the reaction of MeCOSH with a base such cesium carbonate. Typically in such an embodiment, the solvent is N,N-dimethylformamide.

In one embodiment of the fifth aspect of the invention, the reaction step (c) is carried out at a temperature in the range from 0 to 70° C. Typically, the reaction of step (c) is carried out at a temperature in the range from 10 to 60° C. More typically, the reaction of step (c) is carried out at a temperature in the range from 15 to 50° C.

Typically in accordance with the fifth aspect of the invention, in step (c) the N-protected-4-derivatised piperidine (H) or (H′) is present in or added to the solvent at an initial concentration of from 0.01 to 10 mol/L relative to the total volume of solvent used in the reaction mixture. More typically, the N-protected-4-derivatised piperidine (H) or (H′) is present in or added to the solvent at an initial concentration of from 0.1 to 2.0 mol/L. Most typically the N-protected-4-derivatised piperidine (H) or (H′) is present in or added to the solvent at an initial concentration of from 0.5 to 0.8 mol/L.

Typically, the process of step (c) of the fifth aspect of the invention uses from 0.9 to 3.0 molar equivalents of R⁴COS⁻ or MeCOS⁻, relative to the initial amount of the N-protected-4-derivatised piperidine (H) or (H′). More typically, the process uses from 1.0 to 2.0 molar equivalents of R⁴COS⁻ or MeCOS⁻. Most typically, the process uses from 1.4 to 1.6 molar equivalents of R⁴COS⁻ or MeCOS⁻.

Typically, where the process of step (c) of the fifth aspect of the invention employs a base, the process uses from 0.9 to 3.0 molar equivalents of the base, relative to the initial amount of the N-protected-4-derivatised piperidine (H) or (H′). More typically, the process uses from 1.0 to 2.0 molar equivalents of the base. Most typically, the process uses from 1.4 to 1.6 molar equivalents of the base.

In one embodiment of the fifth aspect of the invention, the process of step (c) comprises the steps of:
(i) combining the N-protected-4-derivatised piperidine (H) or (H′) with a solvent to form a first mixture;
(ii) adding the base to the mixture formed in step (i) to form a second mixture; and
(iii) adding the R⁴COSH or MeCOSH to the mixture formed in step (ii) to form a third mixture.

In one embodiment of the fifth aspect of the invention, at the end of the reaction the process of step (c) further comprises the work-up steps of:
(i) optionally filtering off solids from the reaction mixture to provide a filtrate;
(ii) washing the reaction mixture or the filtrate with one or more aqueous washes, optionally with the addition of a further water immiscible solvent such as ethyl acetate, wherein the N-protected-4-(acylthio)-piperidine (I) or (I′) is retained in the organic phase;
(iii) optionally drying the organic phase over a sulfate such as magnesium sulfate or sodium sulfate; and
(iv) optionally removing solvent from the organic phase under vacuum.

Typically the process of step (c) comprises all four of work-up steps (i) to (iv).

In one embodiment the one or more aqueous washes comprise washes with (i) water, (ii) aqueous sodium bicarbonate solution, and (iii) aqueous sodium chloride solution.

An eighth aspect of the invention provides an N-protected-4-(acylthio)-piperidine (I) or a salt thereof:

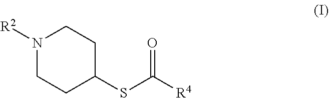

wherein R² is a nitrogen protecting group and R⁴ is a C₁-C₂₀ hydrocarbyl group, wherein the C₁-C₂₀ hydrocarbyl group may be straight-chained or branched, or be or include one or more cyclic groups, wherein the C₁-C₂₀ hydrocarbyl group may optionally be substituted with one or more oxo (═O) and/or one or more halo groups, and wherein the C₁-C₂₀ hydrocarbyl group may optionally include one or more heteroatoms independently selected from N, O and S in its carbon skeleton.

In the eighth aspect of the invention, R² and R⁴ may be as defined in accordance with any embodiment of the fifth aspect of the invention.

A particular embodiment of the eighth aspect of the invention provides benzyl 4-(acetylthio)piperidine-1-carboxylate (I′) or a salt thereof:

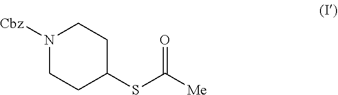 [SMILES: CC(=O)SC1CCN(C(=O)OCc2ccccc2)CC1]

The N-protected-4-(acylthio)-piperidine (I) or the salt thereof, or the benzyl 4-(acetylthio)piperidine-1-carboxylate (I′) or the salt thereof, may be prepared by or preparable by a process of step (c) of the fifth aspect of the invention.

Typically the N-protected-4-(acylthio)-piperidine (I) or the benzyl 4-(acetylthio)-piperidine-1-carboxylate (I′) of the eighth aspect of the invention is in non-salt form.

In one embodiment of the fifth aspect of the invention, the reaction step (d) comprises contacting the N-protected-4-(acylthio)-piperidine (I) with a halogenating agent to form the N-protected-4-(halosulfonyl)-piperidine (J).

In one embodiment the halogenating agent is selected from n-chlorosuccinimide, 1,3-dichloro-5,5-dimethylhydantoin, trichloroisocyanuric acid, C₂, n-bromosuccinimide, 1,3-dibromo-5,5-dimethylhydantoin, tribromoisocyanuric acid and Br₂. Typically, the halogenating agent is selected from N-chlorosuccinimide, 1,3-dichloro-5,5-dimethylhydantoin, trichloroisocyanuric acid, N-bromosuccinimide, 1,3-dibromo-5,5-dimethylhydantoin and tribromoisocyanuric acid. More typically, the halogenating agent is selected from N-chlorosuccinimide and N-bromosuccinimide. Most typically the halogenating agent is N-chlorosuccinimide.

In one embodiment of the fifth aspect of the invention, the N-protected-4-(acylthio)-piperidine (I) is contacted with the halogenating agent in the presence of an acid and an aqueous solvent. In one embodiment, the acid is selected from HCl, HBr, or a carboxylic acid such as formic acid, acetic acid, propionic acid, butyric acid, oxalic acid, malonic acid, succinic acid, tartaric acid, maleic acid or fumaric acid. Typically, the acid is a carboxylic acid, more typically a monocarboxylic acid such as formic acid, acetic acid, propionic acid or butyric acid. Most typically, the acid is acetic acid.

In one embodiment of the fifth aspect of the invention, the aqueous solvent of reaction step (d) is water or a mixture of water and one or more water miscible solvents such as acetonitrile, methanol, ethanol, propanol, acetone, N,N-dimethylformamide, dioxane, or tetrahydrofuran. Typically, the aqueous solvent is water.

In an exemplary embodiment of the fifth aspect of the invention, the reaction step (d) comprises contacting benzyl 4-(acetylthio)piperidine-1-carboxylate (I′) with a chlorinating agent to obtain benzyl 4-(chlorosulfonyl)-1-piperidinecarboxylate (J′):

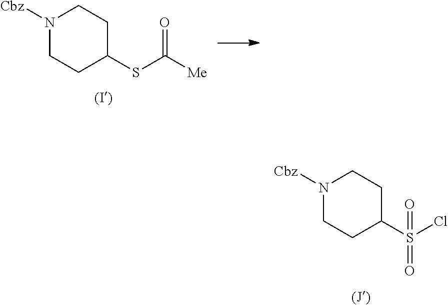 [SMILES: CC(=O)SC1CCN(C(=O)OCc2ccccc2)CC1 ; O=C(OCc1ccccc1)N1CCC(S(=O)(=O)Cl)CC1]

Typically in such an embodiment, the chlorinating agent is N-chlorosuccinimide. Typically in such an embodiment, the benzyl 4-(acetylthio)piperidine-1-carboxylate (I′) is contacted with the chlorinating agent in the presence of acetic acid and water.

In one embodiment of the fifth aspect of the invention, the reaction step (d) is carried out at a temperature in the range from 0 to 50° C. Typically, the reaction of step (d) is carried out at a temperature in the range from 10 to 40° C. More typically, the reaction of step (d) is carried out at a temperature in the range from 15 to 30° C.

Typically in accordance with the fifth aspect of the invention, in step (d) the N-protected-4-(acylthio)-piperidine (I) or (I′) is present in or added to the solvent at an initial concentration of from 0.01 to 2 mol/L relative to the combined total volume of acid and solvent used in the reaction mixture. More typically, the N-protected-4-(acylthio)-piperidine (I) or (I′) is present in or added to the solvent at an initial concentration of from 0.05 to 0.5 mol/L. Most typically the N-protected-4-(acylthio)-piperidine (I) or (I′) is present in or added to the solvent at an initial concentration of from 0.1 to 0.3 mol/L.

Typically, the process of step (d) of the fifth aspect of the invention uses from 1.0 to 5.0 molar equivalents of the halogenating agent, relative to the initial amount of N-protected-4-(acylthio)-piperidine (I) or (I′). More typically, the process uses from 2.0 to 4.0 molar equivalents of the halogenating agent. Most typically, the process uses from 2.5 to 3.0 molar equivalents of the halogenating agent.

Typically, where the process of step (d) of the fifth aspect of the invention employs an acid and an aqueous solvent, the acid comprises from 50 to 99% of the combined total volume of the acid and the solvent. More typically, the acid comprises from 75 to 98% of the combined total volume of the acid and the solvent. More typically still, the acid comprises from 85 to 95% of the combined total volume of the acid and the solvent.

Typically, where the process of step (d) of the fifth aspect of the invention employs an acid and an aqueous solvent, the water comprises from 1 to 50% of the combined total volume of the acid and the solvent. More typically, the water comprises from 2 to 25% of the combined total volume of the acid and the solvent. More typically still, the water comprises from 5 to 15% of the combined total volume of the acid and the solvent.

In one embodiment of the fifth aspect of the invention, the process of step (d) comprises the steps of:
(i) combining the N-protected-4-(acylthio)-piperidine (I) or (I′) with the acid to form a first mixture;
(ii) adding an aqueous solvent such as water to the mixture formed in step (i) to form a second mixture; and
(iii) adding the halogenating agent to the mixture formed in step (ii) to form a third mixture.

In one embodiment of the fifth aspect of the invention, at the end of the reaction the process of step (d) further comprises the work-up steps of:
(i) extracting the N-protected-4-(halosulfonyl)-piperidine (J) or (J′) into a water immiscible organic solvent such as dichloromethane, to form an organic extract;
(ii) optionally washing the organic extract with one or more aqueous washes, wherein the N-protected-4-(halosulfonyl)-piperidine (J) or (J′) is retained in the organic phase; and
(iii) optionally drying the organic extract over a sulfate such as magnesium sulfate or sodium sulfate.

Typically the process of step (d) comprises all three of work-up steps (i) to (iii).

In one embodiment the one or more aqueous washes comprise washes with (i) water, and (ii) aqueous sodium bicarbonate solution.

A ninth aspect of the invention provides an N-protected-4-(halosulfonyl)-piperidine (J) or a salt thereof:

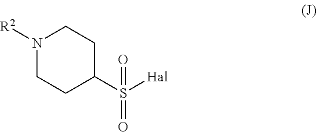

wherein R² is a nitrogen protecting group and Hal is Cl or Br.

In the ninth aspect of the invention, R² and Hal may be as defined in accordance with any embodiment of the fifth aspect of the invention.

A particular embodiment of the ninth aspect of the invention provides benzyl 4-(chlorosulfonyl)-1-piperidinecarboxylate (J′) or a salt thereof:

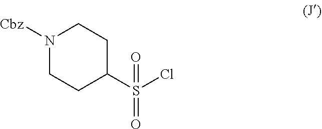 [SMILES: O=C(OCc1ccccc1)N1CCC(S(=O)(=O)Cl)CC1]

The N-protected-4-(halosulfonyl)-piperidine (J) or the salt thereof, or the benzyl 4-(chlorosulfonyl)-1-piperidinecarboxylate (J′) or the salt thereof, may be prepared by or preparable by a process of step (d) of the fifth aspect of the invention.

Typically the N-protected-4-(halosulfonyl)-piperidine (J) or the benzyl 4-(chloro-sulfonyl)-1-piperidinecarboxylate (J′) of the ninth aspect of the invention is in non-salt form.

In one embodiment of the fifth aspect of the invention, the reaction step (e) comprises contacting the N-protected-4-(halosulfonyl)-piperidine (J) with ammonia to form the N-protected-4-piperidinesulfonamide (K).

Typically, the N-protected-4-(halosulfonyl)-piperidine (J) is contacted with ammonia in the presence of a solvent. Typically, the reaction step (e) comprises purging a solution of the N-protected-4-(halosulfonyl)-piperidine (J) with ammonia gas.

Typically, the solvent is a polar aprotic solvent such as dimethyl sulfoxide, N,N-dimethylformamide, N,N′-dimethylpropyleneurea, tetrahydrofuran, 1,4-dioxane, ethyl acetate, acetone, acetonitrile, dichloromethane, hexamethylphosphoramide, nitromethane, propylene carbonate, N-methyl pyrrolidone, or a mixture thereof. Typically the solvent does not comprise an ester. More typically the solvent does not comprise a carbonyl group. For example, the solvent may be selected from dimethyl sulfoxide, tetrahydrofuran, 1,4-dioxane, acetonitrile, dichloromethane, hexamethylphosphoramide, nitromethane, or a mixture thereof. More typically still, the solvent does not comprise a carbonyl, C═N or C≡N group. For example, the solvent may be selected from dimethyl sulfoxide, tetrahydrofuran, 1,4-dioxane, dichloromethane, hexamethylphosphoramide, nitromethane, or a mixture thereof. Most typically, the solvent is dichloromethane.

In an exemplary embodiment of the fifth aspect of the invention, the reaction step (e) comprises contacting benzyl 4-(chlorosulfonyl)-1-piperidinecarboxylate (J′) with ammonia to obtain 1-(benzyloxycarbonyl)-4-piperidinesulfonamide (K′):

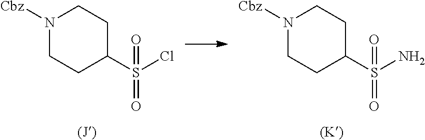 [SMILES: O=C(OCc1ccccc1)N1CCC(S(=O)(=O)Cl)CC1 ; NS(=O)(=O)C1CCN(C(=O)OCc2ccccc2)CC1]

Typically in such an embodiment, the benzyl 4-(chlorosulfonyl)-1-piperidine-carboxylate (J′) is contacted with ammonia in the presence of a polar aprotic solvent such as dichloromethane.

In one embodiment of the fifth aspect of the invention, in step (e) the N-protected-4-(halosulfonyl)-piperidine (J) or (J′) is combined with ammonia at a temperature in the range from −70 to 0° C. Typically the N-protected-4-(halosulfonyl)-piperidine (J) or (J′) is combined with ammonia at a temperature in the range from −50 to −20° C., more typically in the range from −40 to −30° C.

In one embodiment of the fifth aspect of the invention, in step (e) after the N-protected-4-(halosulfonyl)-piperidine (J) or (J′) has been combined with ammonia, the reaction mixture is allowed to warm to a temperature in the range from 10 to 50° C.

Typically, the reaction mixture is allowed to warm to a temperature in the range from to 40° C., more typically to a temperature in the range from 25 to 30° C.

Typically in accordance with the fifth aspect of the invention, in step (e) the N-protected-4-(halosulfonyl)-piperidine (J) or (J′) is present in or added to the solvent at an initial concentration of from 0.01 to 10 mol/L relative to the total volume of solvent used in the reaction mixture. More typically, the N-protected-4-(halosulfonyl)-piperidine (J) or (J′) is present in or added to the solvent at an initial concentration of from 0.1 to 1.0 mol/L. Most typically the N-protected-4-(halosulfonyl)-piperidine (J) or (J′) is present in or added to the solvent at an initial concentration of from 0.2 to 0.4 mol/L.

In one embodiment of the fifth aspect of the invention, at the end of the reaction the process of step (e) further comprises the work-up steps of:
(i) filtering off solids from the reaction mixture to provide a filtrate;
(ii) optionally drying the filtrate over a sulfate such as magnesium sulfate or sodium sulfate; and
(iii) optionally removing solvent from the filtrate under vacuum.

Typically the process of step (e) comprises all four of work-up steps (i) to (iii).

Optionally, the N-protected-4-piperidinesulfonamide (K) or (K′) is isolated by precipitation or crystallisation from a crystallisation solvent. Typically the crystallisation solvent comprises a mixture of polar aprotic and non-polar solvents, such as ethyl acetate and hexanes.

Optionally, the N-protected-4-piperidinesulfonamide (K) or (K′) undergoes one or more purification steps selected from:
(i) treating a solution of the N-protected-4-piperidinesulfonamide (K) or (K′) with neutral alumina; and
(ii) precipitating or crystallising the N-protected-4-piperidinesulfonamide (K) or (K′) from a recrystallisation solvent.

Typically, the purification of the N-protected-4-piperidinesulfonamide (K) or (K′) comprises both purification steps (i) and (ii).

In one embodiment of the fifth aspect of the invention, in purification step (i) the solvent is a mixture of polar aprotic and polar protic solvents, such as a mixture of dichloromethane and methanol.

Typically, after treatment the neutral alumina is removed by filtration.

In one embodiment of the fifth aspect of the invention, in purification step (ii) the recrystallisation solvent is a mixture of polar aprotic, polar protic and non-polar solvents, such as a mixture of dichloromethane, methanol and hexanes. Typically, where the purification comprises both steps (i) and (ii), the recrystallisation solvent is formed by adding a non-polar solvent to the filtrate from step (i).

A tenth aspect of the invention provides an N-protected-4-piperidinesulfonamide (K) or a salt thereof:

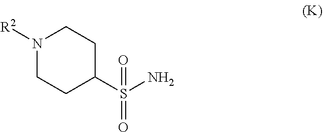

wherein R² is a nitrogen protecting group.

In the tenth aspect of the invention, R² may be as defined in accordance with any embodiment of the fifth aspect of the invention.

A particular embodiment of the tenth aspect of the invention provides 1-(benzyloxycarbonyl)-4-piperidinesulfonamide (K′) or a salt thereof:

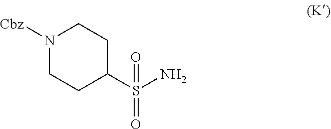 [SMILES: NS(=O)(=O)C1CCN(C(=O)OCc2ccccc2)CC1]

The N-protected-4-piperidinesulfonamide (K) or the salt thereof, or the 1-(benzyloxycarbonyl)-4-piperidinesulfonamide (K′) or the salt thereof, may be prepared by or preparable by a process of step (e) of the fifth aspect of the invention.

Typically the N-protected-4-piperidinesulfonamide (K) or the 1-(benzyloxycarbonyl)-4-piperidinesulfonamide (K′) of the tenth aspect of the invention is in non-salt form.

In one embodiment of the tenth aspect of the invention, the N-protected-4-piperidinesulfonamide (K) or the salt thereof has a HPLC purity of ≥90%. More typically, the N-protected-4-piperidinesulfonamide (K) or the salt thereof has a HPLC purity of ≥95%. More typically still, the N-protected-4-piperidinesulfonamide (K) or the salt thereof has a HPLC purity of ≥96%.

In another embodiment of the tenth aspect of the invention, the 1-(benzyloxycarbonyl)-4-piperidinesulfonamide (K′) or the salt thereof has a HPLC purity of ≥90%. More typically, the 1-(benzyloxycarbonyl)-4-piperidinesulfonamide (K′) or the salt thereof has a HPLC purity of ≥95%. More typically still, the 1-(benzyloxycarbonyl)-4-piperidinesulfonamide (K′) or the salt thereof has a HPLC purity of ≥96%.

In one embodiment of the fifth aspect of the invention, the reaction step (f) comprises the steps of:
(i) de-protecting the N-protected-4-piperidinesulfonamide (K) to form piperidine-4-sulfonamide; and
(ii) alkylating the piperidine-4-sulfonamide to form 1-ethyl-4-piperidine-sulfonamide (A).

As will be understood, the reaction conditions for the de-protection step (i) will correspond to the nitrogen protecting group being removed. For example, where R² is a benzyloxycarbonyl (CBz), 4-methoxy-benzyloxycarbonyl, benzyl, —CH₂R²⁰ or —COOCH₂R²⁰ group it may be removed by catalytic hydrogenolysis or by treatment with HBr in a carboxylic acid such as acetic or trifluoroacetic acid. Where R² is a t-butoxycarbonyl (Boc) group, it may be removed under acidic conditions, e.g. by treatment with trifluoroacetic acid. Where R² is a 2-(4-biphenylyl)-isopropoxycarbonyl (Bpoc) or triphenylmethyl (Trt) group, it may be removed under acidic conditions, e.g. by treatment with trifluoroacetic acid, or by catalytic hydrogenolysis. Where R² is a 2,2,2-trichloroethoxycarbonyl (Troc) group, it may be removed by treatment with zinc in acetic acid. Conditions suitable for deprotection may be found by reference to e.g. Wuts, ‘Greene's Protective Groups in Organic Synthesis’, 5^{th} Ed., 2014, the contents of which are incorporated herein by reference in their entirety.

Typically in accordance with the fifth aspect of the invention, R² is a nitrogen protecting group that may be removed by catalytic hydrogenolysis. Where the nitrogen protecting group is removed by catalytic hydrogenolysis, typically the process of step (i) comprises contacting the N-protected-4-piperidinesulfonamide (K) with a catalyst in the presence of hydrogen gas. Suitable catalysts include Raney nickel and palladium catalysts. In one embodiment, the catalyst is a palladium catalyst, for example palladium on carbon or palladium hydroxide on carbon. Typically, the catalyst is palladium hydroxide on carbon. Typically, the hydrogen gas is used at a pressure in the range from 0.1 to 5 Bar. In one embodiment, the hydrogen gas is used at a typical pressure in the range from 0.5 to 2 Bar, and more typically in the range from 0.8 to 1.2 Bar. In another embodiment, the hydrogen gas is used at a typical pressure in the range from 2 to 4 Bar, and more typically in the range from 2.5 to 3.5 Bar.

Typically, the N-protected-4-piperidinesulfonamide (K) is contacted with the catalyst in the presence of hydrogen gas and a solvent. Typically, the solvent is a polar protic solvent, or a polar aprotic solvent, or a mixture thereof. For example, the solvent may be selected from tetrahydrofuran, 1,4-dioxane, acetonitrile, dichloromethane, water, methanol, ethanol, isopropanol, butanol, or a mixture thereof.

Typically, the catalytic hydrogenolysis of step (i) is carried out at a temperature in the range from 0 to 70° C. In one embodiment of the fifth aspect of the invention, the catalytic hydrogenolysis of step (i) of reaction step (f) is carried out at a temperature in the range from 0 to 50° C. Typically in such an embodiment, the catalytic hydrogenolysis of step (i) is carried out at a temperature in the range from 10 to 35° C. More typically, the catalytic hydrogenolysis of step (i) is carried out at a temperature in the range from 15 to 25° C. In another embodiment of the fifth aspect of the invention, the catalytic hydrogenolysis of step (i) of reaction step (f) is carried out at a temperature in the range from 10 to 50° C. Typically in such an embodiment, the catalytic hydrogenolysis of step (i) is carried out at a temperature in the range from 15 to 30° C.

The alkylation step (ii) of reaction step (f) may be performed under a variety of conditions.

In one embodiment, the alkylation step (ii) comprises contacting the piperidine-4-sulfonamide with Et-X^{f}, wherein X^{f} is a leaving group. Typically in such an embodiment, X^{f} is selected from Cl, Br, I, or a sulphonate leaving group such as a toluenesulfonate, methanesulfonate, or trifluoromethanesulfonate leaving group. More typically, X^{f} is selected from Cl, Br or I.

In one embodiment, the piperidine-4-sulfonamide is contacted with Et-X^{f} in the presence of a solvent and optionally a base. Typically the solvent is a polar aprotic solvent such as dimethyl sulfoxide, N,N-dimethylformamide, N,N′-dimethylpropyleneurea, tetrahydrofuran, 1,4-dioxane, ethyl acetate, acetone, acetonitrile, dichloromethane, hexamethylphosphoramide, nitromethane, propylene carbonate, N-methyl pyrrolidone, or a mixture thereof. Typically the base is a carbonate base, such as an alkali metal or alkali earth metal carbonate.

In another embodiment, the piperidine-4-sulfonamide is alkylated by reductive alkylation. For example, the piperidine-4-sulfonamide may be contacted with acetonitrile or acetaldehyde in the presence of a hydride source such as NaCNBH₃.

Alternatively, the piperidine-4-sulfonamide may be contacted with acetonitrile or acetaldehyde in the presence of a catalyst and hydrogen gas. Typically, the piperidine-4-sulfonamide is contacted with acetonitrile in the presence of a catalyst and hydrogen gas. Suitable catalysts include Raney nickel and palladium catalysts. In one embodiment, the catalyst is a palladium catalyst, for example palladium on carbon or palladium hydroxide on carbon. Typically, the catalyst is palladium hydroxide on carbon. In another embodiment, the catalyst is Raney nickel. Typically, the hydrogen gas is used at a pressure in the range from 0.1 to 5 Bar. In one embodiment, the hydrogen gas is used at a typical pressure in the range from 0.5 to 2 Bar, and most typically in the range from 0.8 to 1.2 Bar. In another embodiment, the hydrogen gas is used at a typical pressure in the range from 2 to 4 Bar, and more typically in the range from 2.5 to 3.5 Bar. Where the piperidine-4-sulfonamide is contacted with acetonitrile or acetaldehyde, in one embodiment the acetonitrile or acetaldehyde, or a mixture of the acetonitrile or acetaldehyde with water, is used as the solvent.

In another embodiment, where the piperidine-4-sulfonamide is contacted with acetonitrile or acetaldehyde, the contact takes place in the presence of a solvent. Typically the solvent is a polar protic solvent, or a polar aprotic solvent (other than acetonitrile or acetaldehyde), or a mixture thereof. For example, the solvent may be selected from tetrahydrofuran, 1,4-dioxane, dichloromethane, water, methanol, ethanol, isopropanol, butanol, or a mixture thereof. More typically, the solvent is a polar protic solvent such as water, methanol, ethanol, isopropanol, butanol, or a mixture thereof. Most typically the solvent is a mixture of ethanol and water. Typically in such an embodiment, from 1 to 10 molar equivalents of acetonitrile or acetaldehyde are used, relative to the amount of piperidine-4-sulfonamide. More typically, from 1.2 to 5 molar equivalents of acetonitrile or acetaldehyde are used. Most typically, from 1.5 to 3.5 molar equivalents of acetonitrile or acetaldehyde are used.

In one embodiment of the fifth aspect of the invention, the alkylation of step (ii) is carried out at a temperature in the range from 0 to 50° C. Typically, the alkylation of step (ii) is carried out at a temperature in the range from 10 to 35° C. More typically, the alkylation of step (ii) is carried out at a temperature in the range from 15 to 25° C.

In another embodiment of the fifth aspect of the invention, the alkylation of step (ii) is carried out at a temperature in the range from 0 to 60° C. Typically in such an embodiment, the alkylation of step (ii) is carried out at a temperature in the range from to 50° C. In one aspect of such an embodiment, the alkylation of step (ii) is carried out at a temperature in the range from 35 to 45° C. In another aspect of such an embodiment, the alkylation of step (ii) is carried out at a temperature in the range from to 30° C.

As will be appreciated, advantageously where R² is a nitrogen protecting group that may be removed by catalytic hydrogenolysis, the steps of:
(i) de-protecting the N-protected-4-piperidinesulfonamide (K) to form piperidine-4-sulfonamide; and
(ii) alkylating the piperidine-4-sulfonamide to form 1-ethyl-4-piperidine-sulfonamide (A),
   may be performed simultaneously or sequentially in a one-pot reaction.

Accordingly, in one embodiment of the fifth aspect of the invention, where R² is a nitrogen protecting group that may be removed by catalytic hydrogenolysis, the reaction step (f) comprises contacting the N-protected-4-piperidinesulfonamide (K) with acetonitrile or acetaldehyde in the presence of a catalyst and hydrogen gas, to obtain 1-ethyl-4-piperadinesulfonamide (A). Typically in such an embodiment, the reaction step (f) comprises contacting the N-protected-4-piperidinesulfonamide (K) with acetonitrile in the presence of a catalyst and hydrogen gas. Suitable catalysts include Raney nickel and palladium catalysts. In one embodiment, the catalyst is a palladium catalyst, for example palladium on carbon or palladium hydroxide on carbon. Typically, the catalyst is palladium hydroxide on carbon.

In an exemplary embodiment of the fifth aspect of the invention, the reaction step (f) comprises contacting 1-(benzyloxycarbonyl)-4-piperidinesulfonamide (K′) with acetonitrile or acetaldehyde in the presence of a catalyst and hydrogen gas, to obtain 1-ethyl-4-piperadinesulfonamide (A):

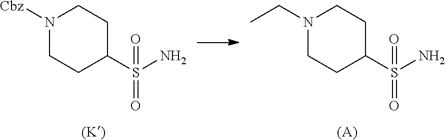 [SMILES: NS(=O)(=O)C1CCN(C(=O)OCc2ccccc2)CC1 ; CCN1CCC(S(N)(=O)=O)CC1 ; "(A)"]

Typically in such an embodiment, the 1-(benzyloxycarbonyl)-4-piperidinesulfonamide (K′) is contacted with acetonitrile in the presence of a catalyst and hydrogen gas. Typically the catalyst is a palladium catalyst such as palladium hydroxide on carbon.

In either of the above two embodiments, where the catalyst is palladium on carbon or palladium hydroxide on carbon, typically from 5-35 wt. % palladium or palladium hydroxide on carbon is used. More typically, from 10-30 wt. % palladium or palladium hydroxide on carbon is used. Most typically, from 15-25 wt. % palladium or palladium hydroxide on carbon is used.

Where reaction step (f) comprises contacting the N-protected-4-piperidinesulfonamide (K) or (K′) with acetonitrile or acetaldehyde in the presence of a catalyst and hydrogen gas, typically the hydrogen gas is used at a pressure in the range from 0.1 to 5 Bar, more typically in the range from 0.5 to 2 Bar, and most typically in the range from 0.8 to 1.2 Bar.

Where reaction step (f) comprises contacting the N-protected-4-piperidinesulfonamide (K) or (K′) with acetonitrile or acetaldehyde in the presence of a catalyst and hydrogen gas, the reaction step (f) may be carried out at a temperature in the range from 0 to 50° C. Typically, the reaction step (f) is carried out at a temperature in the range from 10 to ° C. More typically, the reaction step (f) is carried out at a temperature in the range from 15 to 25° C.

Where the N-protected-4-piperidinesulfonamide (K) or (K′) is contacted with acetonitrile or acetaldehyde, typically the acetonitrile or acetaldehyde, or a mixture of the acetonitrile or acetaldehyde with water, is used as the solvent. In one embodiment, acetonitrile or a mixture of acetonitrile and water is used as the solvent. Typically, a mixture of acetonitrile and water is used as the solvent.

Where a mixture of acetonitrile and water is used as the solvent in step (f), typically solvent mixture comprises from 25 to 50 wt. % water, based on the total weight of the solvent. More typically, the solvent mixture comprises from 30 to 45 wt. % water. Most typically, the solvent mixture comprises from 35 to 40 wt. % water.

Typically in accordance with the fifth aspect of the invention, in step (f) the N-protected-4-piperidinesulfonamide (K) or (K′) is present in or added to the solvent at an initial concentration of from 0.01 to 10 mol/L relative to the total volume of solvent used in the reaction mixture. More typically, the N-protected-4-piperidinesulfonamide (K) or (K′) is present in or added to the solvent at an initial concentration of from 0.1 to 1.0 mol/L. Most typically the N-protected-4-piperidinesulfonamide (K) or (K′) is present in or added to the solvent at an initial concentration of from 0.3 to 0.5 mol/L.

In one embodiment of the fifth aspect of the invention, where reaction step (f) comprises contacting the N-protected-4-piperidinesulfonamide (K) or (K′) or piperidine-4-sulfonamide with acetonitrile or acetaldehyde in the presence of a catalyst and hydrogen gas, at the end of the reaction the process of step (f) further comprises the work-up steps of:
(i) removing the hydrogen;
(ii) removing the catalyst, e.g. by filtration;
(iii) optionally decolourising the reaction mixture using activated charcoal;
(iv) optionally contacting the reaction mixture with a metal scavenger such as SiliaMetS thiol; and
(v) optionally removing the reaction solvent under vacuum, e.g. by co-evaporation with an alcohol such as n-butanol, to obtain the 1-ethyl-4-piperadine-sulfonamide (A).

Typically the process of step (f) comprises all five of work-up steps (i) to (v).

In another embodiment of the fifth aspect of the invention, where R² is a nitrogen protecting group that may be removed by catalytic hydrogenolysis, the reaction step (f) comprises the steps of:
(i) contacting the N-protected-4-piperidinesulfonamide (K) with a first catalyst in the presence of hydrogen gas and a solvent to form an intermediate mixture comprising piperidine-4-sulfonamide and the solvent; and
(ii) contacting the intermediate mixture comprising piperidine-4-sulfonamide and the solvent with acetonitrile or acetaldehyde in the presence of a second catalyst and hydrogen gas, to obtain 1-ethyl-4-piperidine-sulfonamide (A).

In an exemplary embodiment of the fifth aspect of the invention, the reaction step (f) comprises the steps of:
(i) contacting the 1-(benzyloxycarbonyl)-4-piperidinesulfonamide (K′) with a first catalyst in the presence of hydrogen gas and a solvent to form an intermediate mixture comprising piperidine-4-sulfonamide and the solvent; and
(ii) contacting the intermediate mixture comprising piperidine-4-sulfonamide and the solvent with acetonitrile or acetaldehyde in the presence of a second catalyst and hydrogen gas, to obtain 1-ethyl-4-piperidine-sulfonamide (A).

In either of the above two embodiments, the first catalyst and the second catalyst may the same or different. Suitable catalysts include Raney nickel and palladium catalysts.

In one embodiment, the first catalyst and the second catalyst are different. In one aspect of such an embodiment, the first catalyst is a palladium catalyst, for example palladium on carbon or palladium hydroxide on carbon. Typically in such an embodiment, the first catalyst is palladium on carbon. Typically in such an embodiment, the second catalyst is Raney nickel.

The inventors of the present application have found that using a palladium catalyst such as palladium on carbon as the first catalyst and Raney nickel as the second catalyst may be advantageous, since it surprisingly allows for a lower amount and/or a lower carbon loading level of the more expensive palladium catalyst to be used. Typically, only about half the amount of palladium catalyst or half the loading level is required if Raney nickel is used as the second catalyst, versus the use of the palladium catalyst for both steps. Moreover, the use of a lower amount of palladium catalyst renders removal of said catalyst from the reaction mixture more facile.

Where palladium on carbon or palladium hydroxide on carbon is used as the first catalyst and Raney nickel is used as the second catalyst, typically from 2-30 wt. % palladium or palladium hydroxide on carbon is used as the first catalyst. More typically, from 3-20 wt. % palladium or palladium hydroxide on carbon is used as the first catalyst. Most typically, from 5-10 wt. % palladium or palladium hydroxide on carbon is used as the first catalyst.

Where the first catalyst and the second catalyst are different, in one embodiment the first catalyst is removed, e.g. by filtration and/or centrifugation, prior to contacting the intermediate mixture with the acetonitrile or acetaldehyde and the second catalyst. As will be understood, the piperidine-4-sulfonamide may be retained in the intermediate mixture, typically in solution, thus avoiding isolation of the piperidine-4-sulfonamide.

Alternately, the first catalyst may be retained in the reaction mixture, prior to contacting the intermediate mixture with the acetonitrile or acetaldehyde and the second catalyst. Thus, in such an embodiment, the second catalyst and the acetonitrile or acetaldehyde may be added to the intermediate mixture comprising the piperidine-4-sulfonamide, the solvent and the first catalyst.

In another embodiment, the first catalyst and the second catalyst are the same. In one aspect of such an embodiment, the first and the second catalyst is a palladium catalyst, for example palladium on carbon or palladium hydroxide on carbon. Typically in such an embodiment, the first and the second catalyst is palladium hydroxide on carbon. Where the first catalyst and the second catalyst are the same, a first portion of the catalyst may be added to the reaction mixture prior to step (i) and a second portion of the catalyst may be added to the intermediate mixture after step (i), prior to step (ii). Alternately, a single portion of the catalyst may be added to the reaction mixture prior to step (i) and used for both steps (i) and (ii).

Where palladium on carbon or palladium hydroxide on carbon is used as the first and the second catalyst, typically from 5-35 wt. % palladium or palladium hydroxide on carbon is used. More typically, from 10-30 wt. % palladium or palladium hydroxide on carbon is used. Most typically, from 15-25 wt. % palladium or palladium hydroxide on carbon is used.

Typically, where reaction step (f) comprises steps (i) and (ii) discussed above, step (ii) of reaction step (f) comprises contacting the intermediate mixture comprising piperidine-4-sulfonamide and the solvent with acetonitrile in the presence of the second catalyst and hydrogen gas.

Where step (i) of reaction step (f) comprises contacting the N-protected-4-piperidinesulfonamide (K) or (K′) with a first catalyst in the presence of hydrogen gas and a solvent, typically the hydrogen gas is used at a pressure in the range from 0.1 to 5 Bar, more typically in the range from 2 to 4 Bar, and most typically in the range from 2.5 to 3.5 Bar.

Where step (ii) of reaction step (f) comprises contacting the intermediate mixture comprising piperidine-4-sulfonamide and the solvent with acetonitrile or acetaldehyde in the presence of a second catalyst and hydrogen gas, typically the hydrogen gas is used at a pressure in the range from 0.1 to 5 Bar, more typically in the range from 2 to 4 Bar, and most typically in the range from 2.5 to 3.5 Bar.

The hydrogen pressure used in steps (i) and (ii) of reaction step (f) may be the same or different. Typically, the hydrogen pressure used in steps (i) and (ii) of reaction step (f) is the same.

Where reaction step (f) comprises contacting the N-protected-4-piperidinesulfonamide (K) or (K′) with a first catalyst in the presence of hydrogen gas and a solvent, step (i) of reaction step (f) may be carried out at a temperature in the range from 0 to 70° C. Typically, step (i) of reaction step (f) is carried out at a temperature in the range from to 50° C. More typically, step (i) of reaction step (f) is carried out at a temperature in the range from 15 to 30° C.

Where reaction step (f) comprises contacting the intermediate mixture comprising piperidine-4-sulfonamide and the solvent with acetonitrile or acetaldehyde in the presence of a second catalyst and hydrogen gas, step (ii) of reaction step (f) may be carried out at a temperature in the range from 0 to 60° C. Typically, step (ii) of reaction step (f) is carried out at a temperature in the range from 10 to 50° C. In one embodiment, for example when Raney nickel is used as the second catalyst, step (ii) of reaction step (f) is carried out at a temperature in the range from 35 to 45° C. In another embodiment, for example when a palladium catalyst is used as the second catalyst, step (ii) of reaction step (f) is carried out at a temperature in the range from 15 to 30° C.

The temperature ranges used for steps (i) and (ii) of reaction step (f) may be the same or different. Typically, where the first catalyst and the second catalyst are the same, the temperature ranges used for steps (i) and (ii) of reaction step (f) are the same.

Typically the solvent used for steps (i) and (ii) of reaction step (f) is a polar protic solvent, or a polar aprotic solvent (other than acetonitrile or acetaldehyde), or a mixture thereof. For example, the solvent may be selected from tetrahydrofuran, 1,4-dioxane, dichloromethane, water, methanol, ethanol, isopropanol, butanol, or a mixture thereof. More typically, the solvent is a polar protic solvent such as water, methanol, ethanol, isopropanol, butanol, or a mixture thereof. More typically still, the solvent is a mixture of an alcohol (solvent such as methanol, ethanol, isopropanol or butanol) and water. Most typically the solvent is a mixture of ethanol and water.

Where the solvent is a mixture of an alcohol and water, such as a mixture of ethanol and water, typically the alcohol:water ratio is from 90:10 to 10:90 (v/v). More typically, the alcohol:water ratio is from 80:20 to 30:70 (v/v). More typically still, the alcohol:water ratio is from 80:20 to 40:60 (v/v).

In one embodiment, where the solvent is a mixture of an alcohol and water, such as a mixture of ethanol and water, additional water is added to the solvent after step (i), prior to step (ii). For example, additional water may be added such that in step (i) the alcohol:water ratio is from 80:20 to 60:40 (v/v), and in step (ii) the alcohol:water ratio is from 65:35 to 45:55 (v/v).

Where reaction step (f) comprises step (ii) of contacting the intermediate mixture comprising piperidine-4-sulfonamide and the solvent with acetonitrile or acetaldehyde in the presence of a second catalyst and hydrogen gas, typically from 1 to 10 molar equivalents of acetonitrile or acetaldehyde are used, relative to the amount of piperidine-4-sulfonamide. More typically, from 1.2 to 5 molar equivalents of acetonitrile or acetaldehyde are used. Most typically, from 1.5 to 3.5 molar equivalents of acetonitrile or acetaldehyde are used.

The inventors of the present application have surprisingly found that the reductive alkylation reaction proceeds successfully, using such low amounts of acetaldehyde or more especially acetonitrile. This is in contrast to the simultaneous one-pot procedure outlined above where the acetonitrile or acetaldehyde is used as the reaction solvent and so is present in vast excess. Use of low amounts of acetonitrile for example avoids the generation of significant quantities of amines and/or ammonia. Moreover, use of a low defined amount of acetonitrile or acetaldehyde permits monitoring of the reaction via analysis of hydrogen consumption.

Typically, where reaction step (f) comprises steps (i) and (ii), in step (i) the N-protected-4-piperidinesulfonamide (K) or (K′) is present in or added to the solvent at an initial concentration of from 0.01 to 10 mol/L relative to the total volume of solvent used in the reaction mixture of step (i). More typically, the N-protected-4-piperidinesulfonamide (K) or (K′) is present in or added to the solvent at an initial concentration of from 0.1 to 1.0 mol/L. Most typically the N-protected-4-piperidinesulfonamide (K) or (K′) is present in or added to the solvent at an initial concentration of from 0.4 to 0.6 mol/L.

Typically, where reaction step (f) comprises steps (i) and (ii), in step (ii) the piperidine-4-sulfonamide is present in the solvent at an initial concentration of from 0.01 to 10 mol/L relative to the total volume of solvent used in the reaction mixture of step (ii). More typically, the piperidine-4-sulfonamide is present in the solvent at an initial concentration of from 0.1 to 1.0 mol/L. Most typically the piperidine-4-sulfonamide is present in the solvent at an initial concentration of from 0.3 to 0.5 mol/L.

In one embodiment of the fifth aspect of the invention, where reaction step (f) comprises steps (i) and (ii), the reaction step (f) further comprises the work-up steps of:
(iii) removing the hydrogen;
(iv) removing the catalyst(s), e.g. by filtration;
(v) optionally decolourising the reaction mixture using activated charcoal;
(vi) optionally contacting the reaction mixture with a metal scavenger such as SiliaMetS thiol; and
(vii) optionally removing the reaction solvent under vacuum, e.g. by co-evaporation with an alcohol such as n-butanol, to obtain the 1-ethyl-4-piperadine-sulfonamide (A).

Typically, where reaction step (f) comprises steps (i) and (ii), the reaction step (f) further comprises the work-up steps of:
(iii) removing the hydrogen;
(iv) removing the catalyst(s), e.g. by filtration;
(v) optionally decolourising the reaction mixture using activated charcoal; and
(vi) removing the reaction solvent under vacuum, e.g. by co-evaporation with an alcohol such as n-butanol, to obtain the 1-ethyl-4-piperadine-sulfonamide (A).

Optionally, the 1-ethyl-4-piperadinesulfonamide (A) produced by any process of step (f) is purified by precipitation or crystallisation from a crystallisation solvent. Typically the crystallisation solvent comprises a polar aprotic solvent, such as ethyl acetate, or a mixture of polar protic and polar aprotic solvents, such as a mixture of n-butanol and ethyl acetate.

An eleventh aspect of the invention provides 1-ethyl-4-piperadinesulfonamide (A) or a salt thereof:

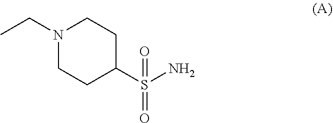

The 1-ethyl-4-piperadinesulfonamide (A) or the salt thereof may be prepared by or preparable by a process of step (f) of the fifth aspect of the invention.

Typically the 1-ethyl-4-piperadinesulfonamide (A) of the eleventh aspect of the invention is in non-salt form.

In one embodiment of the eleventh aspect of the invention, the 1-ethyl-4-piperadine-sulfonamide (A) or the salt thereof has a ¹H NMR purity of ≥95%. More typically, the 1-ethyl-4-piperadinesulfonamide (A) or the salt thereof has a ¹H NMR purity of ≥98.5 In one embodiment of the eleventh aspect of the invention, the 1-ethyl-4-piperadine-sulfonamide (A) or the salt thereof has a GC purity of ≥95%. More typically, the 1-ethyl-4-piperadinesulfonamide (A) or the salt thereof has a GC purity of ≥99%. More typically still, the 1-ethyl-4-piperadinesulfonamide (A) or the salt thereof has a GC purity of ≥99.5% or ≥99.7%.

In one specific embodiment of the fifth aspect of the present invention, there is provided a process of preparing 1-ethyl-4-piperidinesulfonamide (A) or a salt thereof:

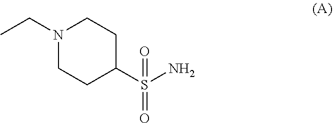

comprising the steps:
(a) converting 4-hydroxy piperidine (F) to N-carboxybenzyl-4-hydroxy piperidine (G′):

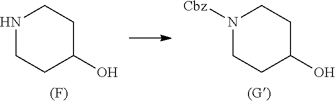

(b) converting N-carboxybenzyl-4-hydroxy piperidine (G′) to benzyl 4-((methylsulfonyl)oxy)piperidine-1-carboxylate (H′):

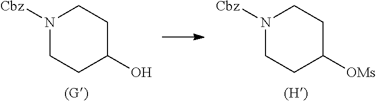

(c) converting benzyl 4-((methylsulfonyl)oxy)piperidine-1-carboxylate (H′) to benzyl 4-(acetylthio)piperidine-1-carboxylate (I′):

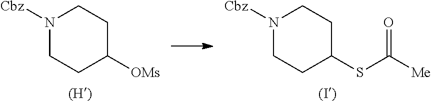

(d) converting benzyl 4-(acetylthio)piperidine-1-carboxylate (I′) to benzyl 4-(chlorosulfonyl)-1-piperidinecarboxylate (J′):

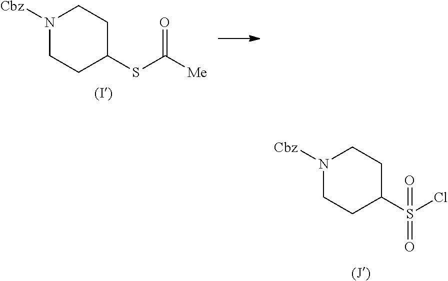

(e) converting benzyl 4-(chlorosulfonyl)-1-piperidinecarboxylate (J′) to 1-(benzyloxycarbonyl)-4-piperidinesulfonamide (K′):

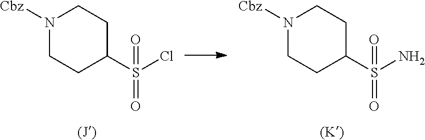 [SMILES: O=C(OCc1ccccc1)N1CCC(S(=O)(=O)Cl)CC1 ; NS(=O)(=O)C1CCN(C(=O)OCc2ccccc2)CC1]

(f) and converting 1-(benzyloxycarbonyl)-4-piperidinesulfonamide (K′) to 1-ethyl-4-piperadinesulfonamide (A):

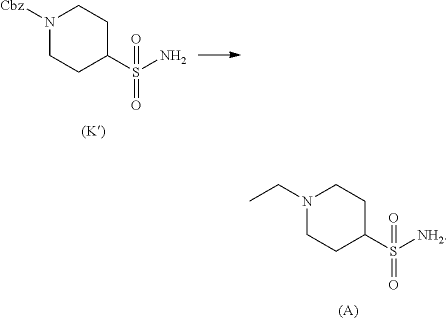 [SMILES: NS(=O)(=O)C1CCN(C(=O)OCc2ccccc2)CC1 ; CCN1CCC(S(N)(=O)=O)CC1 ; "(A)"]

The compounds used in and provided by the present invention can be used both, in their free base form and their acid addition salt form. For the purposes of this invention, a “salt” of a compound of the invention includes an acid addition salt. Acid addition salts are preferably pharmaceutically acceptable, non-toxic addition salts with suitable acids, including but not limited to inorganic acids such as hydrohalogenic acids (for example, hydrofluoric, hydrochloric, hydrobromic or hydroiodic acid) or other inorganic acids (for example, nitric, perchloric, sulfuric or phosphoric acid); or organic acids such as organic carboxylic acids (for example, propionic, butyric, glycolic, lactic, mandelic, citric, acetic, benzoic, salicylic, succinic, malic or hydroxysuccinic, tartaric, fumaric, maleic, hydroxymaleic, mucic or galactaric, gluconic, pantothenic or pamoic acid), organic sulfonic acids (for example, methanesulfonic, trifluoromethanesulfonic, ethanesulfonic, 2-hydroxyethanesulfonic, benzenesulfonic, toluene-p-sulfonic, naphthalene-2-sulfonic or camphorsulfonic acid) or amino acids (for example, ornithinic, glutamic or aspartic acid). The acid addition salt may be a mono-, di-, tri- or multi-acid addition salt. A preferred salt is a hydrohalogenic, sulfuric, phosphoric or organic acid addition salt. A preferred salt is a hydrochloric acid addition salt.

Where a compound of the invention includes a quaternary ammonium group, typically the compound is used in its salt form. The counter ion to the quaternary ammonium group may be any pharmaceutically acceptable, non-toxic counter ion. Examples of suitable counter ions include the conjugate bases of the protic acids discussed above in relation to acid addition salts.

The compounds used in and provided by the present invention can also be used both, in their free acid form and their salt form. For the purposes of this invention, a “salt” of a compound of the present invention includes one formed between a protic acid functionality (such as a carboxylic acid group or a urea group) of a compound of the present invention and a suitable cation. Suitable cations include, but are not limited to lithium, sodium, potassium, magnesium, calcium and ammonium. The salt may be a mono-, di-, tri- or multi-salt. Preferably the salt is a mono- or di-lithium, sodium, potassium, magnesium, calcium or ammonium salt. More preferably the salt is a mono- or di-sodium salt or a mono- or di-potassium salt.

Preferably any salt is a pharmaceutically acceptable non-toxic salt. However, in addition to pharmaceutically acceptable salts, other salts are included in the present invention, since they have potential to serve as intermediates in the purification or preparation of other, for example, pharmaceutically acceptable salts, or are useful for identification, characterisation or purification of the free acid or base.

The compounds and/or salts used in and provided by the present invention may be anhydrous or in the form of a hydrate (e.g. a hemihydrate, monohydrate, dihydrate or trihydrate) or other solvate. Such other solvates may be formed with common organic solvents, including but not limited to, alcoholic solvents e.g. methanol, ethanol or isopropanol.

The compounds, salts and solvates used in and provided by the present invention may contain any stable isotope including, but not limited to ¹²C, ¹³C, ¹H, ²H (D), ¹⁴N, ¹⁵N, ¹⁶O, ¹⁷O, ¹⁸O, ¹⁹F and ¹²⁷I, and any radioisotope including, but not limited to ¹¹C, ¹⁴C, ³H (T), ¹³N, ¹⁵O, ¹⁸F, ¹²³I, ¹²⁴I, ¹²⁵I and ¹³¹I.

Unless stated otherwise, the compounds, salts and solvates used in and provided by the present invention may be in any polymorphic or amorphous form.

A twelfth aspect of the present invention provides a pharmaceutical composition comprising the 1-ethyl-N-((1,2,3,5,6,7-hexahydro-s-indacen-4-yl)carbamoyl)-piperidine-4-sulfonamide or the salt thereof of the second aspect of the invention, and a pharmaceutically acceptable excipient.

Conventional procedures for the selection and preparation of suitable pharmaceutical formulations are described in, for example, “Aulton's Pharmaceutics—The Design and Manufacture of Medicines”, M. E. Aulton and K. M. G. Taylor, Churchill Livingstone Elsevier, 4^{th} Ed., 2013. Pharmaceutically acceptable excipients including adjuvants, diluents or carriers that may be used in the pharmaceutical compositions of the invention, are those conventionally employed in the field of pharmaceutical formulation.

A thirteenth aspect of the present invention provides the 1-ethyl-N-((1,2,3,5,6,7-hexahydro-s-indacen-4-yl)carbamoyl)piperidine-4-sulfonamide or the salt thereof of the second aspect of the invention, or the pharmaceutical composition of the twelfth aspect of the invention, for use in medicine, and/or for use in the treatment or prevention of a disease, disorder or condition.

Most especially, where 1-ethyl-N-((1,2,3,5,6,7-hexahydro-s-indacen-4-yl)-carbamoyl)piperidine-4-sulfonamide is used in the treatment or prevention of a disease, disorder and condition, the 1-ethyl-N-((1,2,3,5,6,7-hexahydro-s-indacen-4-yl)-carbamoyl)piperidine-4-sulfonamide acts as an NLRP3 inhibitor.

In one embodiment, the disease, disorder or condition to be treated or prevented is selected from:
(i) inflammation;
(ii) an auto-immune disease;
(iii) cancer;
(iv) an infection;
(v) a central nervous system disease;
(vi) a metabolic disease;
(vii) a cardiovascular disease;
(viii) a respiratory disease;
(ix) a liver disease;
(x) a renal disease;
(xi) an ocular disease;
(xii) a skin disease;
(xiii) a lymphatic condition;
(xiv) a psychological disorder;
(xv) pain; and
(xvi) any disease where an individual has been determined to carry a germline or somatic non-silent mutation in NLRP3.

Typically, the treatment or prevention of the disease, disorder or condition comprises the administration of the 1-ethyl-N-((1,2,3,5,6,7-hexahydro-s-indacen-4-yl)carbamoyl)-piperidine-4-sulfonamide or the salt thereof of the second aspect of the invention, or the pharmaceutical composition of the twelfth aspect of the invention, to a subject.

Any of the medicaments employed in the present invention can be administered by oral, parenteral (including intravenous, subcutaneous, intramuscular, intradermal, intratracheal, intraperitoneal, intraarticular, intracranial and epidural), airway (aerosol), rectal, vaginal or topical (including transdermal, buccal, mucosal and sublingual) administration.

Typically, the mode of administration selected is that most appropriate to the disorder, disease or condition to be treated or prevented.

A fourteenth aspect of the invention provides a method of inhibiting NLRP3, the method comprising the use of the 1-ethyl-N-((1,2,3,5,6,7-hexahydro-s-indacen-4-yl)-carbamoyl)piperidine-4-sulfonamide or the salt thereof of the second aspect of the invention, or the pharmaceutical composition of the twelfth aspect of the invention, to inhibit NLRP3.

For the avoidance of doubt, insofar as is practicable any embodiment of a given aspect of the present invention may occur in combination with any other embodiment of the same aspect of the present invention. In addition, insofar as is practicable it is to be understood that any preferred, typical or optional embodiment of any aspect of the present invention should also be considered as a preferred, typical or optional embodiment of any other aspect of the present invention.

### EXAMPLES

All solvents, reagents and compounds were purchased and used without further purification unless stated otherwise.

### Abbreviations

Cbz: carboxybenzyl/benzyloxycarbonyl
SAc: acetylthio

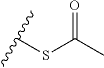 [SMILES: *SC(C)=O]

GC: gas chromatography
HPLC: high performance liquid chromatography
THF: tetrahydrofuran
RBF: round bottom flask
MTBE: methyl tertiary butyl ether
DCM: dichloromethane
DMFL dimethylformamide
TEA: triethylamine
HDPE: high density polyethylene
NMT: No more than
Vol: volumes
AKX reagent: AQUAMICRON® AKX
% a/a: (area under peak of compound (a)/combined area under peaks of compound (a) and all other components)×100

As used herein, unless stated otherwise all references to a pressure in bar refers to the absolute pressure.

### EXPERIMENTAL METHODS

NMR Methods:

NMR spectra were obtained on Bruker AV 400 MHz spectrometer (model: Advance HID) operated at room temperature (25° C.).

GC Methods:

GC analysis was conducted on one of the following machines: Agilent 7890, 6890, or Agilent 6890N with ALS injector.

HPLC Methods:

HPLC in reaction scheme 3 was run using ammonium acetate in water: MeCN (for both mobile phases) on Agilent 1100, 1200, or 1260.

HPLC in reaction scheme 1, steps (i) and (ii), and reaction scheme 2, steps (i)-(iv) was run on run on Waters Alliance e2695 HPLC with PDA detector using 10 Mm ammonium bicarbonate in water as mobile phase-A and acetonitrile as mobile phase-B.

KF Methods:

Coulometric KF (Karl Fischer) titration was run using AKX reagent on Mitsubishi CA-20 or Predicta OM1000.

### SYNTHESIS EXAMPLES

### 1-ethyl-4-piperidinesulfonamide (7)

1-ethyl-4-piperidinesulfonamide (7) was prepared according to the reaction sequence illustrated in reaction scheme 1.

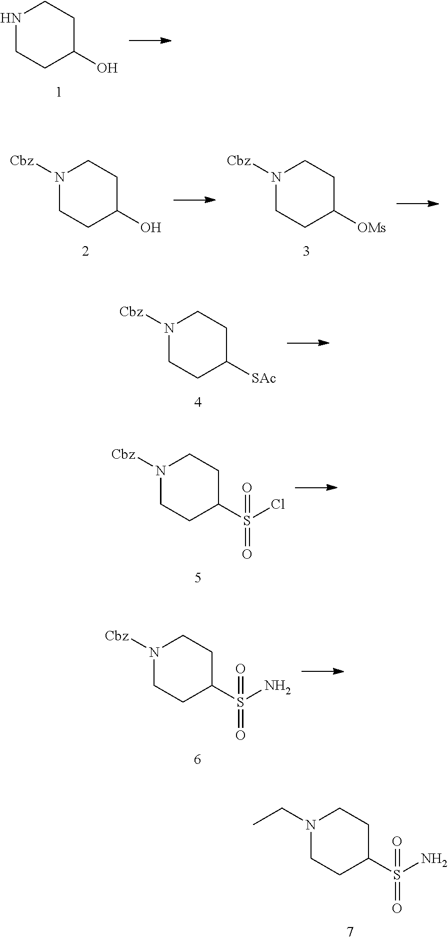

Reaction Scheme 1—Step (i)

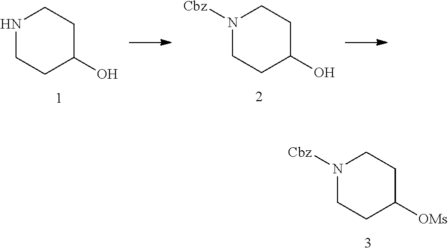 [SMILES: OC1CCNCC1 ; O=C(OCc1ccccc1)N1CCC(O)CC1 ; CS(=O)(=O)OC1CCN(C(=O)OCc2ccccc2)CC1]

Methanol (138.0 L) was charged into a clean and dry four neck RBF (equipped with a mechanical stirrer, nitrogen inlet, thermo pocket and reflux condenser) under nitrogen atmosphere and heated to reflux at 60 to 65° C. for 20-30 min. The temperature was reduced to 25 to 30° C., the refluxed methanol was unloaded and the RBF was rinsed with methanol (23.0 L) and dried under nitrogen and vacuum.

4-hydroxy piperidine (1) (46.0 Kg) was charged into the RBF at 25 to 30° C. 1,4-dioxane (226.0 L) was charged to the RBF at 25 to 30° C. The reaction mixture was stirred for 5-10 minutes and then cooled to 15 to 20° C. A 2N NaOH solution (prepared by mixing NaOH (18.4 Kg) with cold purified water (230.0 L) at 25 to 30° C. in a separate RBF) was slowly charged to the reaction mixture at 15 to 25° C. The reaction mixture was stirred for 5-10 minutes. 50% benzyl chloroformate in toluene (147.2 L) was slowly added over a period of 1-2 hours to the reaction mixture. The temperature was raised to to 30° C. and stirred for 1-2 hours.

A sample of the reaction mixture was analysed by GC for the presence of 4-hydroxy piperidine (1). GC, % a/a: Limit: NMT 10%. Sampling procedure: Take 2 mL of reaction mass add 4 ml water, 2 ml ethyl acetate, stir for 2 min, separate and submit the top organic layer (ethyl acetate) for GC % a/a.

Purified water (230.0 L) was added to the reaction mixture and the reaction mixture was stirred for 10-15 min at 25 to 30° C. MTBE (230.0 L) was charged into the RBF at to 35° C. The reaction mixture was stirred for 15-20 minutes at 25 to 30° C. and then allowed to settle for 20-30 minutes. The organic layer (OL-1) and aqueous layer (AL-1) were separated into different containers and AL-1 was charged back into the RBF. MTBE (230.0 L) was charged into the RBF at 25 to 30° C. The reaction mixture was stirred for 15-20 minutes at 25 to 30° C. and then allowed to settle for 20-30 minutes. The organic layer (OL-2) and aqueous layer (AL-2) were separated into different containers. OL-1 and OL-2 were combined and charged into the RBF at 25 to 30° C. Purified water (138.0 L) was charged to the RBF at 25 to 30° C. The reaction mixture was stirred for 15-20 minutes at 25 to 30° C. and then allowed to settle for 20-30 minutes. The aqueous layer (AL-3) was separated from the organic layer (OL-3).

10% NaCl solution (prepared by adding NaCl (13.80 Kg) to purified water (138.0 L) in a RBF at 25 to 30° C. with stirring) was charged to OL-3 at 25 to 30° C. The reaction mixture was stirred for 15-20 minutes at 25 to 30° C. and then allowed to settle for 20-30 minutes. The organic layer (OL-4) and aqueous layer (AL-4) were separated into different containers. OL-4 was dried with sodium sulfate (23.0 Kg). OL-4 was filtered through a Buchner funnel and washed with MTBE (46.0 L). OL-4 was distilled down to 46-92 L at 40 to 45° C. under vacuum (650 mmHg). The vacuum was released and DCM (138.0 L) was charged to the mixture and the mixture was co-distilled 35 to 40° C. under vacuum to 46-92 L. The mixture was cooled to 25 to 30° C. and the vacuum was released. DCM (552.0 L) was charged to the mixture at 25 to 30° C. and the mixture was stirred for 5-10 minutes. The reaction mixture was cooled to 20 to 25° C. TEA (127.8 L) was added at 20 to 25° C. The reaction mixture was cooled to −5 to 5° C.

Methane sulfonyl chloride (67.62 Kg) was slowly charged at −5 to 5° C. over a period of 1-2 hours. The reaction mixture was raised to 25 to 30° C. and stirred for 1-2 hours at 25 to 30° C.

A sample of the reaction mixture was analysed by HPLC for presence of benzyl 4-hydroxy-1-piperidinecarboxylate (2). HPLC, % a/a: (Limit: NMT 3.0%). Sampling procedure: Take 5 mL of reaction mass add 5 ml water, separate and submit the bottom organic layer (DCM) for HPLC % a/a.

The unwanted salts were filtered, washed with DCM (92.0 L) at 25 to 30° C. and sucked dry completely under vacuum at 25 to 30° C. The filtrate was charged into a RBF at 25 to 30° C. 10% sodium bicarbonate solution (prepared by adding sodium bicarbonate (23.0 Kg) to purified water (230.0 L) at 25 to 30° C.) was charged to the filtrate at 25 to 30° C. The reaction mixture was stirred for 15-20 minutes at 25 to 30° C. and then allowed to settle for 20-30 minutes. The organic layer (OL-5) and aqueous layer (AL-5) were separated into different containers and OL-5 was charged back into the RBF at 25 to 30° C.

Purified water (230.0 L) was charged into the RBF at 25 to 30° C. The reaction mixture was stirred for 15-20 minutes at 25 to 30° C. and then allowed to settle for 20-30 minutes. The organic layer (OL-6) and aqueous layer (AL-6) were separated into different containers and OL-6 was charged back into the RBF at 25 to 30° C. 10% sodium chloride solution (prepared by adding sodium chloride (11.50 Kg) to the purified water (230.0 L) at 25 to 30° C.) was charged to the RBF at 25 to 30° C. The reaction mixture was stirred for 15-20 minutes at 25 to 30° C. and then allowed to settle for 20-30 minutes.

The organic layer (OL-7) and aqueous layer (AL-7) were separated into different containers. OL-7 was dried with sodium sulfate (23.0 Kg). OL-7 was filtered through a Buchner funnel and washed with DCM (46.0 L). OL-7 was distilled down to 46-92 L at to 45° C. under vacuum (650 mmHg). The vacuum was released and ethyl acetate (92.0 L) was charged to the mixture and the mixture was co-distilled 40 to 45° C. under vacuum to 46-92 L. The mixture was cooled to 30 to 40° C. and the vacuum was released. Ethyl acetate (115.0 L) was charged to the mixture at 30 to 40° C. and the mixture was stirred for 10-15 minutes at 30 to 35° C. Hexane (1150.0 L) was slowly charged to the mixture at 30 to 35° C. and the mixture was stirred for 2-3 hours at 25 to 30° C. The solid was filtered on a nutsche filter under vacuum, washed with hexane (92.0 L) at 25 to 30° C. and sucked dry completely under vacuum at 25 to 30° C. The solid material was dried in a vacuum oven at 30 to 35° C. for 6-8 hours, delumping the material every 3-4 hours.

A dried sample of benzyl 4-((methylsulfonyl)oxy)piperidine-1-carboxylate (3) was analysed for cumulative solvent content by GC (Limit: NMT 10% (hexanes, ethyl acetate). The dried material was unloaded into a clean HDPE container for weighing.

The product was stored at 2-8° C. under nitrogen atmosphere. A sample was sent for analysis.

### Final product: benzyl 4-((methylsulfonyl)oxy)piperidine-1-carboxylate

Off white colour (solid)

Output: 121.87 Kg

Yield: 85.5%

HPLC purity: 94.7%

¹H NMR: (CDCl₃ 400 MHz): δ 1.82-1.86 (m, 2H), δ 1.96-1.97 (m, 2H), δ 3.03 (s, 3H), δ 3.41-3.45 (m, 2H) δ 3.72-3.78 (m, 2H), δ 4.88-4.92 (m, 1H) δ 5.13 (s, 2H), δ 7.26-7.37 (m, 5H)

Reaction Scheme 1—Step (ii)

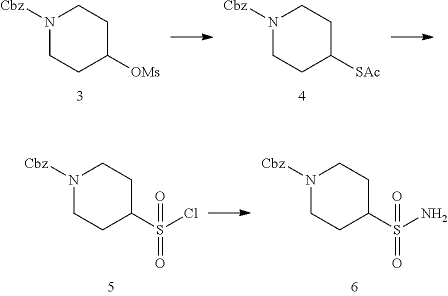

DMF (water content anaylsed by KF (Limit: NMT 0.2% w/v)) was charged in to a clean and dry four neck RBF (equipped with a mechanical stirrer, nitrogen inlet, thermo pocket and reflux condenser) under nitrogen atmosphere and heated to reflux at 60 to 65° C. for 20-30 min. The temperature was reduced to 25 to 30° C., the refluxed DMF was unloaded (water content analysed by KF (Limit: NMT 0.5% w/v)) and the RBF was dried under nitrogen and vacuum.

Benzyl 4-((methylsulfonyl)oxy)piperidine-1-carboxylate (3) (29.0 Kg) was charged to the RBF at 25 to 30° C. DMF (145.0 L) was charged to the RBF at 25 to 30° C. The reaction mixture was stirred for 5-10 minutes, cooled to 15 to 20° C. and then allowed to settle for 20-30 minutes.

Cesium carbonate 44.95 Kg was charged to the RBF at 15 to 25° C. The reaction mixture was stirred for 5-10 minutes. Thio acetic acid 10.56 Kg was charged at 15 to 25° C. (the vent was connected to alkali scrubber/aq KMnO₄). The reaction mixture was raised to to 50° C. and stirred for 24 hours.

A sample of reaction mixture was analysed for benzyl 4-((methylsulfonyl)oxy)-piperidine-1-carboxylate (3) content by HPLC, % a/a: (Limit: NMT 3%). Sampling procedure: Take 2 mL of reaction mass add 4 ml water, 2 ml ethyl acetate stir for 2 min, separate and submit the top organic layer (ethyl acetate) for HPLC % a/a.

The reaction mixture was cooled to 25 to 30° C. The unwanted salts were filtered through a Buchner funnel under vacuum at 25 to 30° C., washed with ethyl acetate (145.0 L) and sucked dry completely under vacuum at 25 to 30° C. The filtrate was charged back to the RBF at 25 to 30° C. and cooled to 15 to 20° C. Purified water (145.0 L) was charged to the RBF at 15-25° C. and the reaction mixture was stirred for 5-10 minutes. Ethyl acetate (145.0 L) was charged to the RBF at 25 to 30° C. The reaction mixture was stirred for 15-20 minutes at 25 to 30° C. and allowed to settle for 20-30 minutes.

The organic layer (OL-1) and aqueous layer (AL-1) were separated into different containers. AL-1 was charged into the RBF at 25 to 30° C. Ethyl acetate (145.0 L) was charged at 25 to 30° C. The reaction mixture was stirred for 15-20 minutes at 25 to 30° C. and allowed to settle for 20-30 minutes.

The organic layer (OL-2) and aqueous layer (AL-2) were separated into different containers. OL-1 and OL-2 were combined and charged into the RBF at 25 to 30° C.

A 10% NaHCO₃ solution (prepared by adding sodium bicarbonate (14.50 Kg) to purified water (145.0 L) at 25 to 30° C. and stirring well to mix) was charged to the RBF at 25 to 30° C. The reaction mixture was stirred for 15-20 minutes at 25 to 30° C. and allowed to settle for 20-30 minutes.

The organic layer (OL-3) and aqueous layer (AL-3) were separated into different containers. OL-3 was charged into the RBF at 25 to 30° C. 10% NaCl solution (prepared by adding NaCl (14.50 Kg) to purified water (145 L) at 25 to 30° C. and stirring well to mix) was charged to the RBF at 25 to 30° C. The reaction mixture was stirred for 15-20 minutes at 25 to 30° C. and allowed to settle for 20-30 minutes.

The organic layer (OL-4) and aqueous layer (AL-4) were separated into different containers. OL-4 was dried with sodium sulfate (14.50 Kg), filtered through a Buchner funnel and washed with ethyl acetate (29.0 L). The filtrate was distilled completely in the RBF until no drops at 45 to 50° C. under vacuum (650 mmHg). The vacuum was released and the mixture was cooled to 25 to 30° C. As sample was analysed for ethyl acetate content by GC (Limit: NMT 20% w/w). Sampling procedure: Take 2 mL crude sample send for HPLC % a/a.

Acetic acid (377.0 L) was charged at 25 to 30° C. to the RBF. The reaction mixture was stirred for 5-10 minutes at 25 to 30° C. Purified water (37.7 L) was charged at 25 to 30° C. The reaction mixture was stirred for 5-10 minutes at 25 to 30° C. and then cooled to 17 to 25° C. N-chlorosuccinimide (33.64 Kg) was slowly added portion wise for 1-2 hours at 18 to 25° C. The reaction mixture was stirred for 1 hour at 25 to 30° C.

A sample was analysed for benzyl 4-(acetylthio)-piperidine-1-carboxylate (4) content by HPLC, % a/a: (Limit: NMT 3%). Sampling procedure: Take 2 mL of reaction mass add 4 ml water, 2 ml DCM stir for 2 min, separate and submit the bottom organic layer (DCM) for HPLC % a/a.

The reaction mixture was cooled to 15 to 20° C. Purified water (377.0 L) was added to the reaction mixture at 15 to 20° C. and the reaction mixture was stirred for 5-10 minutes at 25 to 30° C. DCM (145.0 L) was charged to the RBF at 25 to 30° C. The reaction mixture was stirred for 10-15 minutes at 25 to 30° C. and allowed to settle for 20-30 minutes. The organic layer (OL-5) and aqueous layer (AL-5) were separated into different containers. AL-5 was charged to the RBF. DCM (145.0 L) was charged to the RBF at 25 to 30° C. The reaction mixture was stirred for 10-15 minutes at 25 to 30° C. and allowed to settle for 20-30 minutes.

The organic layer (OL-6) and aqueous layer (AL-6) were separated into different containers. OL-5 and OL-6 were combined and charged into the RBF at 25 to 30° C. Purified water (145.0 L) was charged to the RBF at 25 to 30° C. The reaction mixture was stirred for 5-10 minutes at 25 to 30° C. and allowed to settle for 25-30 minutes.

The organic layer (OL-7) and aqueous layer (AL-7) were separated into different containers. OL-7 was charged to the RBF. Part one of a 2% sodium bicarbonate solution (prepared by adding sodium bicarbonate (8.70 Kg) with purified water (435.0 L) and dividing into three equal volume parts) was charged to the RBF at 25 to 30° C. The reaction mixture was stirred for 5-10 minutes at 25 to 30° C. and allowed to settle for 25-30 minutes.

The organic layer (OL-8) and aqueous layer (AL-8) were separated into different containers. OL-8 was charged to the RBF. Part two of the above 2% sodium bicarbonate solution was charged to the RBF at 25 to 30° C. The reaction mixture was stirred for 5-10 minutes at 25 to 30° C. and allowed to settle for 25-30 minutes.

The organic layer (OL-9) and aqueous layer (AL-9) were separated into different containers. OL-9 was charged to the RBF. Part three of the above 2% sodium bicarbonate solution was charged to the RBF at 25 to 30° C. The reaction mixture was stirred for 5-10 minutes at 25 to 30° C. and allowed to settle for 25-30 minutes.

The organic layer (OL-10) and aqueous layer (AL-10) were separated into different containers. OL-10 was dried with sodium sulfate (14.50 Kg), filtered at 25 to 30° C., and washed with DCM (29.0 L). The filtrate was charged to RBF at 25 to 30° C.

The reaction mixture was cooled to −40 to −30° C. and purged with ammonia gas for 2-3 hours. The temperature was raised to 25 to 30° C. and stirred for 10-12 hours at 25 to 30° C. A sample of the reaction mixture sample was analysed for 1-(benzyloxycarbonyl)-4-piperidinesulfonamide (5) content by HPLC, % a/a: (Limit: NMT 3%). Sampling procedure: Take 2 mL of reaction mass add 4 ml water, separate and submit the bottom organic layer (DCM) for HPLC % a/a.

The unwanted salts were filtered under vacuum at 25 to 30° C., washed with DCM (14.50 L) and sucked dry completely. The filtrate was charged into a clean and dried RBF at 25 to 30° C. and dried with sodium sulfate (14.50 Kg). The mixture was filtered at 25 to 30° C. and the sodium sulfate was washed with DCM (14.50 L). The mixture was charged through a 0.2 micron filter cartridge into a clean and dried RBF and distilled under vacuum at 35 to 40° C. down to 29-58 L.

The vacuum was released and the reaction mixture was cooled to 25 to 30° C. Ethyl acetate (58.0 L) was charged to the RBF at 25 to 30° C. and the mixture was distilled under vacuum at 35 to 40° C. down to 29-58 L. The vacuum was released and the reaction mixture was cooled to 25 to 30° C. Ethyl acetate (72.5 L) was charged to the RBF at 25 to 30° C. and the mixture was stirred for 30 min at 25 to 30° C. Hexane (36.25 L) was charged to the RBF at 25 to 30° C. and the mixture was stirred for 1-2 hours at 25 to 30° C. The solid was filtered under vacuum at 25 to 30° C., washed with hexane (58.0 L) and sucked dry completely. A wet sample was anaylsed for HPLC purity % a/a.

Output: 11.0 Kg

Yield: 39.85%

HPLC purity: 90.5%

Purification

Wet material from four batches of reaction scheme 1, step (ii) (53.95 Kg) was charged into a clean and dry RBF at 25 to 30° C. DCM (580 L) was charged at 25 to 30° C. and the mixture was stirred for 5-10 minutes at 25 to 30° C. Methanol (25.0 L) was charged at to 30° C. and the mixture was stirred for 5-10 minutes at 25 to 30° C. Neutral alumina (174.0 Kg) was charged at 25 to 30° C. and the mixture was stirred for 1 hour at 25 to 30° C. The neutral alumina was filtered at 25 to 30° C. The salts were washed with DCM (150.0 L). The filtrate was charged in to a clean and dried RBF at 25 to 30° C. Hexane (1050 L) was charged at 25 to 30° C. and the mixture was stirred for 1-2 hours at 25 to 30° C. The precipitate was filtered under vacuum at 25 to 30° C., washed with hexane (116.0 L) and sucked dry completely (until no drops). The wet material was dried under vacuum at 30 to 35° C. for 6-8 hours with delumping every 3 hours). The dried material was unloaded into a clean HDPE container and weighed. The product was stored at 2-8° C. under nitrogen atmosphere. A sample was sent for analysis.

### Final product: 1-(benzyloxycarbonyl)-4-piperidinesulfonamide

White colour (solid powder)

Output: 41.60 Kg

Yield: 41.80%

HPLC purity: 96.1%

¹H NMR: (DMSO 400 MHz): δ 1.41-1.51 (m, 2H), δ 1.99-2.01 (m, 2H), S 2.50-286 (m, 2H), δ 3.022-3.05 (m, 1H) δ 4.08-4.11 (m, 2H), S 5.75 (s, 2H) δ 6.78 (s, 2H), δ 7.40-7.30 (m, 5H)

Reaction Scheme 1—Step (iii)

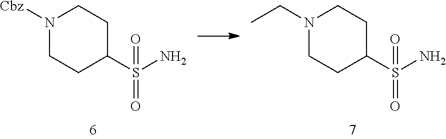 [SMILES: NS(=O)(=O)C1CCN(C(=O)OCc2ccccc2)CC1 ; CCN1CCC(S(N)(=O)=O)CC1]

1-(benzyloxycarbonyl)-4-piperidinesulfonamide (6) (21.85 Kg) was charged to a vessel which was then purged with nitrogen. Acetonitrile (free of propionitrile) (109.8 Kg) and purified water (65.0 L) were charged to the vessel and the temperature was adjusted to 15 to 25° C. The vessel was vacuum/nitrogen purged three times at 15 to 25° C. and then charged with palladium hydroxide on carbon (20 wt %; 50% water) (0.455 Kg). The vessel was vacuum/nitrogen purged three times at 15 to 25° C. The vessel was vacuum/hydrogen purged three times at 15 to 25° C. and maintained under an atmosphere of hydrogen (ca. 1 bar absolute). The reaction mixture was stirred until complete. After approximately 1.5 hours reaction time the vessel was purged with vacuum/hydrogen to remove CO₂. Completion was measured by ¹H NMR analysis, pass criterion ≤10.0 mol % 1-(benzyloxycarbonyl)-4-piperidinesulfonamide (6).

The vessel was vacuum/nitrogen purged three times at 15 to 25° C. and then charged with palladium hydroxide on carbon (20 wt %; 50% water) (2.265 Kg) at 15 to 25° C. The vessel was vacuum/nitrogen purged three times at 15 to 25° C. The vessel was vacuum/hydrogen purged three times at 15 to 25° C. and maintained under an atmosphere of hydrogen (ca. 1 bar absolute).

The reaction mixture was stirred at 15 to 25° C. until complete. After approximately 1.5 hours reaction time the vessel was purged with vacuum/hydrogen to remove ammonia. Completion was measured by ¹H NMR analysis, pass criterion ≤5.0 mol % 4-piperidinesulfonamide.

Once the pass criterion by ¹H NMR analysis was met, the reaction mixture was stirred at 15 to 25° C. until complete by GC analysis. Pass criterion ≤0.05% combined area of 4-piperidinesulfonamide plus intermediate at relative retention time: 0.939 intermediate.

Once the reaction was deemed complete by GC, the vessel was purged with nitrogen and the reaction mixture was filtered through a 1 μm filter at 15 to 25° C. to remove the catalyst. The filter cake was twice washed with pre-mixed purified water and acetonitrile (17.5 Kg:22.0 Kg and 17.2 Kg:21.9 Kg) at 15 to 25° C.

The filtrate was charged with decolourising charcoal (activated) (4.40 Kg) and stirred at to 25° C. for at least 60 minutes (target 60 to 120 minutes). The mixture was filtered through a 1 μm filter at 15 to 25° C. to remove the charcoal. The filter cake was washed twice with pre-mixed purified water and acetonitrile (17.4 Kg:22.0 Kg and 17.0 Kg:22.0 Kg) at 15 to 25° C. The filtrate was charged with SiliaMetS Thiol 40-63 m 60 Å (4.515 Kg) and stirred at 15 to 25° C. for at least 60 minutes (target 60 to 120 minutes). The mixture was filtered through a 0.6 μm filter at 15 to 25° C. to remove SiliaMetS Thiol. The filter cake was twice washed with pre-mixed purified water and acetonitrile (18.2 Kg:22.0 Kg and 18.1 Kg:22.0 Kg) at 15 to 25° C.

The filtrate was charged to a vessel and adjusted to 50 to 60° C., concentrated under reduced pressure at 50 to 60° C. to ca 110 L. n-Butanol (89.8 Kg) was charged at 50 to 60° C. and the mixture was concentrated under reduced pressure at 50 to 60° C. to ca 110 L. n-Butanol (86.9 Kg) was charged at 50 to 60° C. and the mixture was concentrated under reduced pressure at 50 to 60° C. to ca 110 L. n-Butanol (88.4 Kg) was charged at 50 to 60° C. and the mixture was concentrated under reduced pressure at 50 to 60° C. to ca 90 L. The supernatant of the concentrated mixture was analysed for water content by KF analysis, pass criterion ≤0.5% w/w water.

The temperature was adjusted to 15 to 25° C. and ethyl acetate (98.6 Kg) was charged at 15 to 25° C. The reaction mixture was cooled to −2 to +2° C. over at least 60 minutes (target 60 to 120 minutes). The mixture was stirred at −2 to 2° C. for at least 4 hours (target 4 to 6 hours). The solid was filtered on 20 μm filter cloth at −2 to 2° C. and washed twice with ethyl acetate, (38.1 Kg and 39.9 Kg) at −2 to 2° C.

The solid was dried at up to 60° C. under a flow of nitrogen until the n-butanol content was ≤0.5% w/w and ethyl acetate content was ≤0.5% w/w (measured by ¹H NMR spectroscopy). The dried weight of the solid 1-ethyl-4-piperidinesulfonamide (7) was measured and assayed using ¹H NMR spectroscopy.

### Final Product: 1-ethyl-4-piperidinesulfonamide

Output: 12.00 Kg

Yield: 85%

GC purity: 99.7%

NMR purity: 98.700

¹H NMR: (DMSO) 0.95 (t), 1.55 (dq), 1.80 (app t), 1.95 (app d), 2.30 (q), 2.75 (m), 2.90 (app d)

Reaction Scheme 1—Step (iii)—Alternative Procedure A

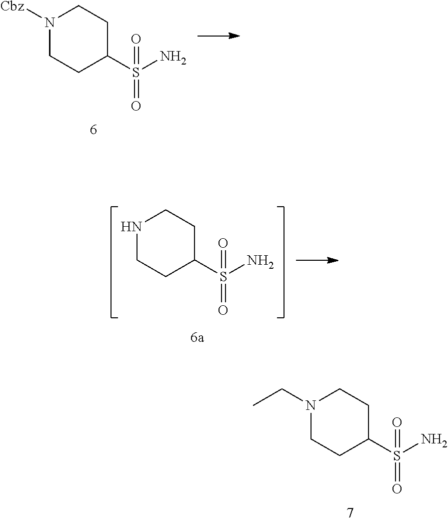

1-(benzyloxycarbonyl)-4-piperidinesulfonamide (6) (20 g) was charged to a vessel and suspended at room temperature in a mixture of ethanol (78.9 g) and purified water (40.0 g). The vessel was purged with a light stream of argon and charged with 10% Pd/C Evonik type Noblyst® P1070 (1.00 g, 53.9% water content) and purged with argon (8 bar) three times at room temperature and then purged with hydrogen (6 bar) five times at room temperature. The vessel was heated to 25±2° C. and maintained under an atmosphere of hydrogen (ca. 3 bar). The reaction mixture was stirred until complete (typically 1 to 2 hours), as judged by the detected consumption of hydrogen. Reaction completion was then measured by GC analysis, pass criterion ≤1.0 relative area % 1-(benzyloxycarbonyl)-4-piperidinesulfonamide (6).

The vessel was purged with argon (8 bar) three times at 25±2° C. and then charged with Raney Nickel (Johnson Matthey Type A-5000) (2.0 g) as a slurry in water (60.0 mL). Acetonitrile (8.26 g) was added and the vessel was purged three times with argon (8 bar) at 25±2° C. The vessel was purged with hydrogen (6 bar) five times at 25±2° C. and then heated to 40±2° C. and maintained under an atmosphere of hydrogen (ca. 3 bar).

The reaction mixture was stirred at 40±2° C. until complete (typically 12 to 18 hours), as judged by the detected consumption of hydrogen. Reaction completion was measured by GC analysis, pass criterion ≤0.05 relative area % 4-piperidinesulfonamide (6a).

Once the reaction was deemed complete by GC analysis, the vessel was purged with argon and the reaction mixture filtered over a glass fibre filter (Macherey-Nagel MN GF-5, porosity 0.4 μm) applying light vacuum. The filter cake was washed two to three times with pre-mixed purified water and ethanol (100 g: 78.9 g) at 25±2° C.

The filtrate was charged to a vessel and concentrated under reduced pressure. n-Butanol (81.0 g) was charged and the mixture was concentrated to residue under reduced pressure. n-Butanol (64.8 g) was charged at room temperature followed by ethyl acetate (90.2 g) and the mixture was cooled from room temperature to 0±5° C. over at least 4 hours.

The resulting solid was filtered over a Buchner funnel with a sintered glass disc (porosity 3) and washed with ethyl acetate (90.2 g) at 0° C.

The solid product was dried at up to 50° C. under a flow of nitrogen for max. 24 hours.

### Final Product: 1-ethyl-4-piperidinesulfonamide (7)

Output: 9.36 g

Yield: 71.3%

GC purity: 98.3%

Reaction Scheme 1—Step (iii)—Alternative Procedure B

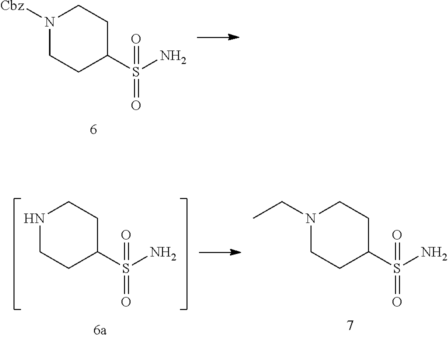

1-(benzyloxycarbonyl)-4-piperidinesulfonamide (6) (21.85 Kg) was charged to a vessel which was then purged with nitrogen. Ethanol (85.2 Kg) and purified water (43.7 L) were charged to the vessel and the temperature was adjusted to 15 to 25° C. The vessel was vacuum/nitrogen purged three times at 15 to 25° C. and then charged with palladium hydroxide on carbon (20 wt %; 50% water) (0.66 Kg). The vessel was vacuum/nitrogen purged three times at 15 to 25° C. The vessel was vacuum/hydrogen purged three times at 15 to 25° C. and maintained under an atmosphere of hydrogen (ca. 3 bar). The reaction mixture was stirred until complete. Completion was measured by ¹H NMR analysis, pass criterion ≤5.0 mol % 1-(benzyloxycarbonyl)-4-piperidinesulfonamide (6).

The vessel was vacuum/nitrogen purged three times at 15 to 25° C. and then charged with palladium hydroxide on carbon (20 wt %; 50% water) (1.09 Kg) as a slurry in water (21.85 Kg) and acetonitrile (9.2 Kg) at 15 to 25° C. The vessel was heated to 35 to 45° C. and vacuum/nitrogen purged three times at 15 to 25° C. The vessel was vacuum/hydrogen purged three times at 15 to 25° C. and maintained under an atmosphere of hydrogen (ca. 3 bar).

The reaction mixture was stirred at 15 to 25° C. until complete. At approximately 6 hours intervals the reaction vessel was purged with vacuum/hydrogen to remove ammonia. Completion was measured by ¹H NMR analysis, pass criterion ≤5.0 mol % 4-piperidinesulfonamide.

Once the pass criterion by ¹H NMR analysis was met, the reaction mixture was stirred at 15 to 25° C. until complete by GC analysis. Pass criterion ≤0.05% combined area of 4-piperidinesulfonamide plus intermediate at relative retention time: 0.939 intermediate.

Once the reaction was deemed complete by GC, the vessel was purged with nitrogen and the reaction mixture cooled to 15 to 25° C. and filtered through a 1 μm filter at 15 to 25° C. to remove the catalyst. The filter cake was twice washed with pre-mixed purified water and ethanol (13.1 Kg:10.9 Kg and 13.1 Kg:10.9 Kg) at 15 to 25° C.

The filtrate was charged with decolourising charcoal (activated) (4.37 Kg) and stirred at to 25° C. for at least 60 minutes (target 60 to 120 minutes). The mixture was filtered through a 1 μm filter at 15 to 25° C. to remove the charcoal. The filter cake was washed twice with pre-mixed purified water and ethanol (13.1 Kg:10.9 Kg and 13.1 Kg:10.9 Kg) at 15 to 25° C.

The filtrate was charged to a vessel and adjusted to 50 to 60° C., concentrated under reduced pressure at 50 to 60° C. to ca 110 L. n-Butanol (89.8 Kg) was charged at 50 to 60° C. and the mixture was concentrated under reduced pressure at 50 to 60° C. to ca 110 L. n-Butanol (86.9 Kg) was charged at 50 to 60° C. and the mixture was concentrated under reduced pressure at 50 to 60° C. to ca 110 L. n-Butanol (88.4 Kg) was charged at 50 to 60° C. and the mixture was concentrated under reduced pressure at 50 to 60° C. to ca 90 L. The supernatant of the concentrated mixture was analysed for water content by KF analysis, pass criterion ≤0.5% w/w water.

The temperature was adjusted to 15 to 25° C. and ethyl acetate (98.6 Kg) was charged at 15 to 25° C. The reaction mixture was cooled to −2 to +2° C. over at least 60 minutes (target 60 to 120 minutes). The mixture was stirred at −2 to 2° C. for at least 4 hours (target 4 to 6 hours). The solid was filtered on 20 μm filter cloth at −2 to 2° C. and washed twice with ethyl acetate, (38.1 Kg and 39.9 Kg) at −2 to 2° C.

The solid was dried at up to 60° C. under a flow of nitrogen until the n-butanol content was ≤0.5% w/w, ethanol content ≤0.5% w/w, and ethyl acetate content was ≤0.5% w/w (measured by ¹H NMR spectroscopy). The dried weight of the solid 1-ethyl-4-piperidinesulfonamide (7) was measured and assayed using ¹H NMR spectroscopy.

### Final Product: 1-ethyl-4-piperidinesulfonamide

Output: 10.98 Kg

Yield: 78%

### 4-(phenoxvcarbonvlamino)-1,2,3,5,6,7-hexahydro-s-indacene (13)

4-(phenoxycarbonylamino)-1,2,3,5,6,7-hexahydro-s-indacene (13) was prepared according to the reaction sequence illustrated in Reaction Scheme 2.

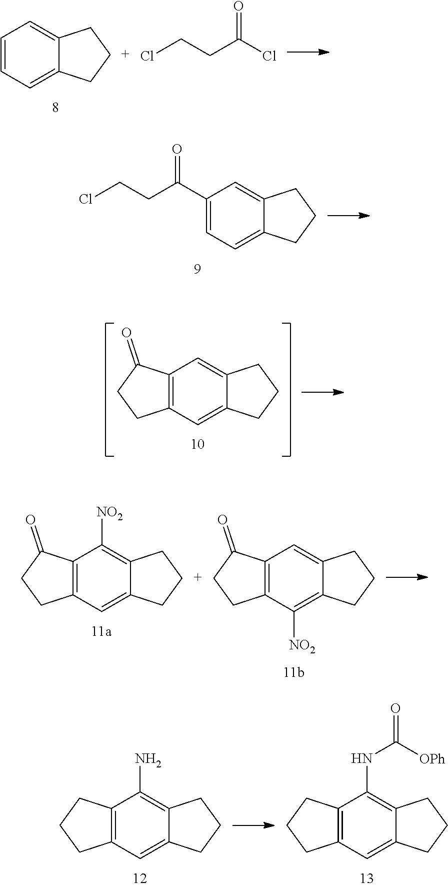

Reaction Scheme 2—Step (i)

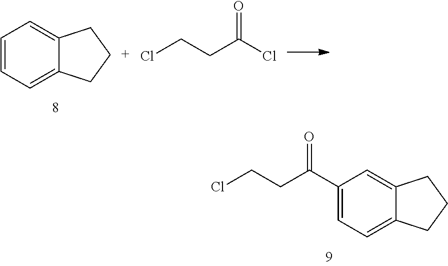

Reagents had methanol content of no more than 0.5% by GC.

DCM (385 L) and AlCl₃ (99.86 Kg) were charged at 25 to 30° C. under a nitrogen atmosphere into a 2.0 KL clean and dry glass-lined reactor. The reaction mixture was cooled to −10° C.

3-chloropropanoyl chloride (90.99 Kg) was added slowly at −10 to −5° C. under a nitrogen atmosphere. The reaction mixture was maintained for 30 minutes at −10° C. under a nitrogen atmosphere. 2,3-dihydro-1H-indene (8) (77.00 Kg was then added slowly to the reaction mixture at −10 to −5° C. under nitrogen atmosphere.

The reaction mixture was maintained for 2 hours at 10 to 15° C. The absence of 2,3-dihydro-1H-indene (8) was confirmed by HPLC (Limit: 5.0%).

After completion of the reaction, the reaction mixture was added slowly to a 6 N hydrochloric acid solution (prepared from water (308 L) and conc. hydrochloric acid (308 L)) at 0 to 10° C. DCM (231 L) was added and the reaction mixture temperature was raised to 30 to 35° C. The reaction mixture was stirred at 30 to 35° C. for 30 minutes and allowed to settle at 30 to 35° C. for 30 minutes. The layers were separated and the organic layer (OL-1) was kept aside. DCM (231 L) was charged to the aqueous layer at 25 to 30° C. The reaction mixture was stirred at 25 to 30° C. for 30 minutes and allowed to settle at 25 to 30° C. for 30 minutes. The layers were separated (aqueous layer (AL-1) and organic layer (OL-2)) and AL-1 was kept aside. OL-1 and OL-2 were combined at 25 to 30° C. Demineralised water (385 L) was added to the combined organic layers. The reaction mixture was stirred at 25 to 30° C. for 30 minutes and allowed to settle at 25 to 30° C. for 30 minutes. The layers were separated (aqueous layer (AL-2) and organic layer (OL-3)) and AL-2 was kept aside.

10% Saturated sodium bicarbonate solution (prepared from demineralised water (385 L) and sodium bicarbonate (38.5 Kg)) was charged to OL-3 at 25 to 30° C. The reaction mixture was stirred at 25 to 30° C. for 30 minutes and allowed to settle at 25 to 30° C. for 30 minutes. The layers were separated (aqueous layer (AL-3) and organic layer (OL-4)) and AL-3 was kept aside. OL-4 was dried over anhydrous Na₂SO₄ (38.5 Kg) and the anhydrous Na₂SO₄ was washed with DCM (150 L) at 25 to 30° C.

The solvent was distilled under vacuum at below 35 to 40° C. until 5% remained. n-hexane (308 L) was charged to the reaction mixture at 35 to 40° C. and the solvent was distilled completely at 35 to 40° C. until no condensate drops were formed. n-hexane (150 L) was charged to the reaction mixture at 35 to 40° C. and the reaction mixture was cooled to 5 to 10° C. and maintained at 5 to 10° C. for 30 minutes.

The solid product was filtered, washed with cooled hexane (77 L), and dried in a hot air oven at 40 to 45° C. for 6 hours to afford the product.

### Final Product: 3-chloro-1-(2,3-dihydro-1H-inden-5-yl)propan-1-one (9)

Output: 120.5 Kg

Yield: 88.63%

HPLC purity: 99.3%

Moisture content: 0.09%

¹H NMR: (500 MHz, CDCl3): δ 7.81 (S, 1H), 7.76 (d, 1H), 7.31 (d, 1H), 3.93 (t, 2H), 3.45 (t, 2H), 2.97 (t, 4H), 2.15 (q, 2H)

Reaction Scheme 2—Step (ii) and Step (iii)

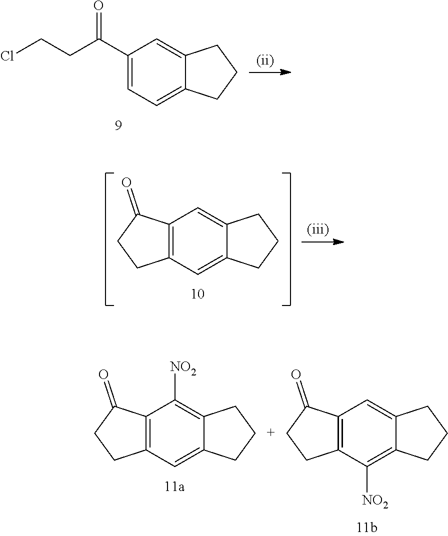

Sulfuric acid (300.0 L) was charged at 25 to 30° C. into a 2.0 KL clean and dry glass-lined reactor. 3-chloro-1-(2,3-dihydro-1H-inden-5-yl)propan-1-one (9) (60.0 Kg) was charged lot wise at 25 to 30° C. and the reaction mixture was maintained for 30 minutes at 25 to 30° C. The reaction mixture was slowly heated to 65 to 70° C. and maintained at 65 to 70° C. for 24 hours. The absence of 3-chloro-1-(2,3-dihydro-1H-inden-5-yl)propan-1-one (9) was confirmed by HPLC (Limit: 1.0%).

Then the reaction mixture was cooled to 0 to 5° C. A nitration mixture*¹ was added slowly at 0 to 5° C. and the reaction mixture was maintained at 0 to 5° C. for 1 hour. The absence of 1,2,3,5,6,7-hexahydro-s-indacen-1-one (10) was confirmed by HPLC (Limit: 1.0%). The reaction mixture was maintained at 0 to 5° C.

Demineralised water (900.0 L) was charged at 25 to 30° C. into a 2.0 KL clean and dry glass-lined reactor. The water was cooled to 0 to 5° C. The reaction mixture was added slowly added to the reactor at 0 to 5° C. Toluene (480.0 L) was added and the temperature was raised to 30 to 35° C. The reaction mixture was maintained at 30 to 35° C. for 30 minutes and allowed to settle at 30 to 35° C. for 30 minutes. The reaction mixture was filtered through a Celite® bed (prepared with Celite® (6.0 Kg) and toluene (30.0 L)). The Celite® bed was washed with toluene (60.0 L). The solid was filtered and sucked dry for 30 min.

The reaction mixture was charged to a 2.0 KL clean and dry glass-lined reactor. The reaction mixture was allowed to settle at 30 to 35° C. for 30 minutes. The layers were separated (aqueous layer (AL-1) and organic layer (OL-1)) and OL-1 was kept aside. Toluene (60.0 L) was charged to AL-1. The reaction mixture was stirred at 35 to 40° C. for 30 minutes and allowed to settle at 35 to 40° C. for 30 minutes. The layers were separated (aqueous layer (AL-2) and organic layer (OL-2)) and OL-2 was kept aside. OL-1 and OL-2 were combined to form OL-3.

A 5% saturated sodium bicarbonate solution (prepared from demineralised water (300.0 L) and sodium bicarbonate (15.0 Kg)) was slowly charged to OL-3 at 30 to 35° C. The reaction mixture was stirred at 35 to 40° C. for 30 minutes and allowed to settle at 35 to 40° C. for 30 minutes. The reaction mixture was filtered through a Celite® bed (prepared with Celite® (6.0 Kg) and demineralised water (60.0 L)). The Celite® bed was washed with toluene (60.0 L).

The reaction mixture was charged to a 3.0 KL clean and dry glass-lined reactor. The reaction mixture was allowed to settle at 30 to 35° C. for 30 minutes. The layers were separated (aqueous layer (AL-3) and organic layer (OL-4)) and OL-4 was kept aside.

Toluene (60.0 L) was charged to AL-3. The layers were separated (aqueous layer (AL-4) and organic layer (OL-5)) and OL-5 was kept aside. OL-4 and OL-5 were combined to form OL-6. Brine solution (prepared from demineralised water (300.0 L) and sodium chloride (12.0 Kg) at 25 to 30° C. The reaction mixture was stirred at 30 to 35° C. for 30 minutes and allowed to settle at 30 to 35° C. for 30 minutes. The layers were separated (aqueous layer (AL-5) and organic layer (OL-7)) and OL-7 was kept aside. OL-7 was dried over anhydrous Na₂SO₄ (9.0 Kg) and the anhydrous Na₂SO₄ was washed with toluene (30.0 L) at 25 to 30° C. The solvent was distilled under vacuum at below 40 to 45° C. until 5% remained. Methanol (60.0 L) was charged to the reaction mixture at 40 to 45° C. and down to 60 L of reaction mass.

Methanol (120.0 L) was charged to the reaction mixture at 40 to 45° C. and the reaction mixture was cooled to 5 to 10° C. and maintained at 5 to 10° C. for 30 minutes. The solid product was filtered, washed with cooled methanol (30.0 L), and dried in a hot air oven at 40 to 45° C. for 6 hours to afford the product.

*1: To prepare the nitration mixture, sulfuric acid (27.0 L) was charged at 25 to 30° C. into a 160 L clean and dry glass-lined reactor. The reaction mixture was cooled to 0 to 5° C. Nitric acid (27.0 L) at 0 to 5° C. was added slowly and the reaction mixture was maintained for 30 minutes at 0 to 5° C. to afford the nitration mixture.

### Final Product: 8-nitro-1,2,3,5,6,7-hexahydro-s-indacen-1-one (11a) and 4-nitro-1,2,3,5,6,7-hexahydro-s-indacen-1-one (11b)

Combined Output (11a+11b): 38.87 Kg

Combined Yield (11a+11b): 62.24%

Weight ratio (11a:11b): 9:1

HPLC purity: 95.9%

Moisture content: 0.19%

¹H NMR: (500 MHz, CDCl₃): δ7.44 (S, 1H), 2.21 (m, 2H), 2.78 (t, 2H), 3.02 (m, 4H), 3.13 (t, 2H)

Reaction Scheme 2—Step (iv)

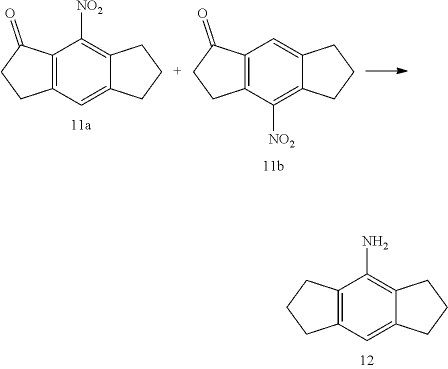

A mixture of 8-nitro-1,2,3,5,6,7-hexahydro-s-indacen-1-one (1a) and 4-nitro-1,2,3,5,6,7-hexahydro-s-indacen-1-one (11b) (9:1 ratio; 27.0 Kg) at 25 to 30° C. was charged into a 600 L clean and dry pressure reactor.

Methanol (270 L) was charged at 25 to 30° C. Methane sulfonic acid (14.3 Kg) was slowly charged at 25 to 30° C. and the reaction mixture was maintained for 30 minutes. 15% Pd(OH)₂ slurry (60% wet)*² was added.

The reaction mixture was degassed under vacuum and filled with an argon atmosphere (0.5 Kg) three times. The reaction mixture was degassed under vacuum and filled with a hydrogen atmosphere (0.5 Kg) three times. Then the reaction mixture was stirred under hydrogen pressure (100 Psi) at room temperature for 32 hours. The temperature was gradually raised up to 55° C. The absence of 8-nitro-1,2,3,5,6,7-hexahydro-s-indacen-1-one (11a) and 4-nitro-1,2,3,5,6,7-hexahydro-s-indacen-1-one (11b) was confirmed by HPLC (Limit: 1.0%).

After completion of the reaction, the reaction mixture was cooled to 25 to 30° C. The reaction mixture was degassed under vacuum and filled with nitrogen atmosphere (0.5 Kg) three times.

The reaction mixture was filtered through a candy filter to remove Pd(OH)₂, followed by a micro filter and the bed was washed with methanol (54 L). 95% of the solvent was distilled off under vacuum at below 45 to 50° C. Demineralised water (135 L) was charged into the reaction mixture at 25 to 30° C. and maintained for 30 minutes. The reaction mixture was cooled to 5-10° C. The pH was adjusted to about 9-10 with 2 N aqueous NaOH solution (prepared from NaOH (6.48 Kg) and demineralised water (81 L)) and the reaction mixture was stirred for 30 minutes. Then toluene (135 L) was charged to the reaction mixture and the reaction mixture was stirred for 30 minutes. The reaction mixture was stirred for a further 30 minutes, whilst bringing the temperature up to 25 to 30° C. The reaction mixture was allowed to settle for 30 minutes, whilst the temperature was maintained at 25 to 30° C.

The reaction mixture was filtered through a Celite® bed (prepared with Celite® (5.4 Kg) and toluene (13.5 L). The Celite® bed was washed with toluene (54 L).

The layers were separated (aqueous layer (AL-1) and organic layer (OL-1)) and OL-1 was kept aside. Toluene (54 L) was added to AL-1 at 25 to 30° C. The reaction mixture was stirred at 25 to 30° C. for 30 minutes and allowed to settle at 25 to 30° C. for 30 minutes. The layers were separated (aqueous layer (AL-2) and organic layer (OL-2)) and AL-2 was kept aside. Toluene (54 L) was added to AL-1 at 25 to 30° C. A brine solution (prepared with demineralised water (135 L) and sodium chloride (54 Kg)) was charged to the combined organic layers (OL-1 and OL-2) at 25 to 30° C. The reaction mixture was stirred at 25 to 30° C. for 30 minutes and allowed to settle at 25 to 30° C. for 30 minutes.

The layers were separated (aqueous layer (AL-3) and organic layer (OL-3)) and AL-3 was kept aside. Charcoal (1.3 Kg) was added to OL-3 and the temperature was raised to 35-40° C. and maintained at 35 to 40° C. for 30 minutes. The reaction mixture was filtered through a Celite® bed (prepared with Celite® (5.4 Kg) and toluene (54 L)) at 35 to 40° C. The Celite® bed was washed with toluene (54 L). The organic layer was dried over anhydrous Na₂SO₄ (13.5 Kg). The Na₂SO₄ was washed with toluene (27 L).

The solvent was distilled under vacuum at below 35 to 40° C. until 5% remained. Methanol (40.5 L) was charged to the reaction mixture at 35 to 40° C. and distilled until 5% remained. Methanol (97.2 L) and water (10.8 L) were charged to the reaction mixture at 35 to 40° C. The reaction mixture was heated to 50 to 55° C., stirred for 1 hour at 50 to 55° C., slowly cooled to 0 to 5° C. and maintained at 0 to 5° C. for 30 minutes.

The solid product was filtered and washed with cold methanol (13.5 L), and dried in a hot air oven at 40 to 45° C. for 6 hours to afford the product.

*2: To prepare the 15% Pd(OH)₂ slurry, 20% Pd(OH)₂ on carbon (60% wet; 4.05 Kg) was added to methanol (27 L).

### Final product: 1,2,3,5,6,7-hexahydro-s-indacen-4-amine (12)

Output: 11.3 Kg

Yield: 41.85%

HPLC purity: 98.1%

Moisture content: 0.10

¹H NMR: (400 MHz, DMSO-d₆): δ 6.38 (S, 1H), 4.45 (S, 2H), 2.75 (t, 4H), 2.58 (t, 4H), 1.98 (t, 4H).

### Purification (A) of 1,2,3,5,6,7-hexahydro-s-indacen-4-amine (12)

1,2,3,5,6,7-Hexahydro-s-indacen-4-amine (12) (54.5 Kg) was charged at 25 to 30° C. into a 250 L clean and dry reactor. Toluene (27.2 L) was charged at 25 to 30° C. and the reaction mixture was stirred at 25 to 30° C. for 30 minutes. Methanol (163 L) was charged to the reaction mixture at 25 to 30° C. The reaction mixture was stirred at 25 to 30° C. for 30 minutes, cooled to −5 to 0° C., and stirred at −5 to 0° C. for 30 minutes. The solid product was filtered, washed with cold methanol (54.5 L), and dried at 40 to 45° C. for 6 hours.

### Final Product: 1,2,3,5,6,7-hexahydro-s-indacen-4-amine (12)

Output: 40.5 Kg

Yield: 74.31%

HPLC purity: 99.5%

Moisture content: 0.3%

¹H NMR: (400 MHz, DMSO-d₆): δ 6.33 (s, 1H), 4.53 (s, 2H), 2.72 (t, 4H), 2.57 (t, 4H), 1.98 (t, 4H).

### Crop Purification (B) of 1,2,3,5,6,7-hexahydro-s-indacen-4-amine (12)

The filtered mother liquors from five batches of reaction scheme 2, step (iv) were combined and concentrated to afford crude 1,2,3,5,6,7-hexahydro-s-indacen-4-amine (12) (25 Kg) and purified through a 100-200 mesh silica gel column. The column was eluted with 5 to 10% ethyl acetate (42 L) in hexane (658 L).

The pure fractions were concentrated under reduced pressure (600 mm of Hg) at 40 to 45° C. to afford crude 1,2,3,5,6,7-hexahydro-s-indacen-4-amine (12) (15 Kg).

Toluene (7.5 L) was added at 25 to 30° C. and the reaction mixture was stirred for 30 minutes at 25 to 30° C. Methanol (45 L) was added at 25 to 30° C. and the reaction mixture was stirred for 30 minutes at 25 to 30° C. The reaction mixture was cooled to −5 to 10° C. and stirred for 30 minutes. Purity was checked using HPLC (Limit 98%, Single max purity: NMT: 1%).

The solid was filtered, washed with cold methanol (15 L) and dried at 40 to 45° C. in vacuum tray drier for 6 hours.

### Final Product: 1,2,3,5,6,7-hexahydro-s-indacen-4-amine (12)

Output: 10.2 Kg

Yield: 9.36%

HPLC purity: 99.3%

Moisture content: 0.12%

¹H NMR: (400 MHz, DMSO-d₆): δ 6.33 (S, 1H), 4.51 (S, 2H), 2.72 (t, 4H), 2.59 (t, 4H), 1.99 (t, 4H).

Combined yield of five batches of reaction scheme 2, step iv including purification (A) and crop purification (B): 46.56%

Reaction Scheme 2—Step (v)

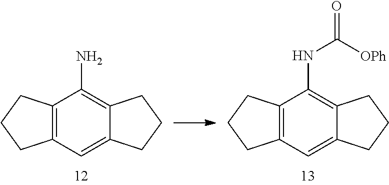 [SMILES: Nc1c2c(cc3c1CCC3)CCC2 ; O=C(Nc1c2c(cc3c1CCC3)CCC2)Oc1ccccc1]

1,2,3,5,6,7-hexahydro-s-indacen-4-amine (12)(7.50 Kg) was charged to a clean and dry reactor. THF (60.05 Kg) was added to the reactor and the temperature was adjusted to between 0 and 10° C. to form a clear brown solution. N,N′-diisopropylethylamine (6.66 Kg) dissolved in THF (6.78 Kg) was charged to the reactor whilst maintaining the temperature between 0 and 10° C. (line rinse with THF (6.78 Kg) at 0 to 10° C.). The temperature was maintained at 0 to 5° C.

Phenyl chloroformate (7.44 Kg) dissolved in THF (6.74 Kg) was charged to the reactor over a minimum of 1 hour whilst maintaining the temperature between 0 and 10° C. to form a slurry (line rinse with THF (6.66 Kg) at 0 to 10° C.). The temperature of the reaction mixture was raised to between 15 and 25° C. and stirred until complete. Completion was measured by ¹H NMR analysis. Pass criterion ≤1.0 mol % 1,2,3,5,6,7-hexahydro-s-indacen-4-amine (12).

The temperature of the reaction mixture was increased to between 30 and 40° C. The reaction mixture was concentrated under reduced pressure to about 37.5 L. Absolute ethanol (31.50 Kg) was charged to the reaction mixture at between 30 and 40° C. The reaction mixture was concentrated under reduced pressure to about 37.5 L. Absolute ethanol (29.60 Kg) was charged to the reaction mixture at between 30 and 40° C. The reaction mixture was concentrated under reduced pressure to about 37.5 L. Absolute ethanol (29.74 Kg) was charged to the reaction mixture at between 30 and 40° C. The reaction mixture was concentrated under reduced pressure to about 37.5 L. Absolute ethanol charging and concentrating was repeated until sample of the reaction mixture passes analysis by ¹H NMR. Pass criterion ≤0.5% w/w THF relative to product.

Absolute ethanol (30.12 Kg) was charged to the reaction mixture at between 15 and 40° C. The reaction mixture was cooled to between 0 and 5° C. and stirred for 45 to 90 minutes. The solid was filtered on a 20 μm filter cloth at 0 to 5° C. The solid was washed with absolute ethanol (11.72 Kg and 12.00 Kg) at 0 to 5° C. and sucked down on the filter for 30 to 90 minutes under nitrogen purge.

The solid was identified and analysed by HPLC. Pass criterion ≤0.5% DIPEA·HCl relative to product. The solid was dried under vacuum at up to 50° C. under a flow of nitrogen until the ethanol content was ≤0.5% w/w.

### Final Product: 4-(phenoxycarbonylamino)-1,2,3,5,6,7-hexahydro-s-indacene (13)

Output: 11.78 Kg

Yield: 93%

HPLC purity: 99.6%

### 1-Ethyl-N-((1,2,3,5,6,7-hexahydro-s-indacen-4-yl)carbamoyl)piperidine-4-sulfonamide (potassium salt) (14)

Reaction Scheme 3

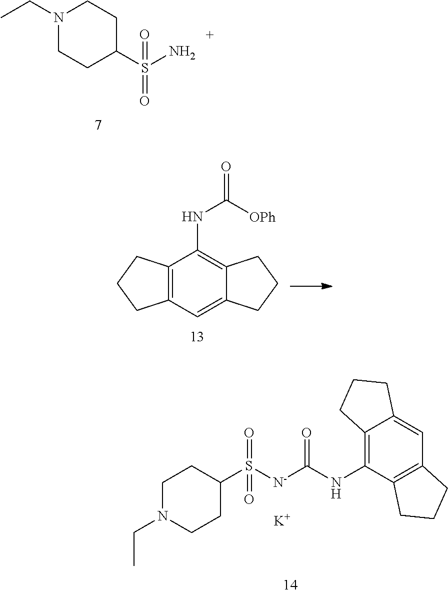

1-ethyl-4-piperidinesulfonamide (7) (7.85 Kg) was charged to a vessel. Dimethyl sulphoxide (33.5 Kg) was charged to the vessel and the mixture was adjusted to 20 to 25° C. The mixture was stirred for at least 60 minutes (target 60 to 90 minutes) at 20 to 25° C. until full solution was obtained. Potassium tert-butoxide (5.1 Kg) was charged in at least six portions to the vessel over at least 60 minutes (target 60 to 90 minutes) maintaining the temperature at 20 to 30° C. (target 20 to 25° C.). The mixture was adjusted to 20 to 25° C. and stirred for at least 30 minutes (target 30 to 60 minutes) at to 25° C.

4-(phenoxycarbonylamino)-1,2,3,5,6,7-hexahydro-s-indacene (13) (12.55 Kg) was charged in at least six portions to the vessel over at least 30 minutes (target 30 to 90 minutes) maintaining the temperature at 20 to 30° C. The reaction mixture was stirred at 20 to 30° C. for at least 60 minutes or until reaction completed. A sample was analysed for completion by ¹H NMR. Pass criterion ≤5.0 mol % 1-ethyl-4-piperidinesulfonamide (7), taking a consecutive passing sample.

The reaction mixture was weighed in a separate container and then transferred back to the vessel using a line rinse of dimethyl sulphoxide (17.2 Kg). The mixture was stirred and adjusted to 20 to 25° C. The water content was analysed by KF.

Acetonitrile (62.0 Kg) was charged to the vessel over at least 30 minutes maintaining the temperature at 20 to 25° C. Water (3.00 Kg) was charged to the vessel over 2-3 hours maintaining the temperature at 20 to 25° C. Acetonitrile (19.4 Kg) was charged to the vessel maintaining the temperature at 20 to 25° C. The mixture was stirred for at least 1 hour (target 1 to 3 hours) at 20 to 25° C. The mixture was cooled to 0 to 5° C. over at least 1 hour (target 1 to 2 hours), stirred for at least 1 hour (target 1 to 4 hours) at 0 to 5° C., filtered over 1 to 2 μm cloth at 0 to 5° C. and the filter cake was washed with pre-mixed (6:13:0.4) dimethyl sulfoxide/acetonitrile/water (5.34 Kg:8.32 Kg:0.31 Kg) at 0 to 5° C.

The solid was dried under vacuum for ca. 2 hours until suitable for handling and the filter cake was analysed for water content by KF. Pass criterion ≤5.5% w/w.

The filter cake was slurry washed with acetonitrile (62.3 Kg) at 15 to 25° C. for 30 to 60 minutes before filtering at 15 to 25° C. The filter cake was washed with acetonitrile (19.6 Kg) at 15 to 25° C. The filter cake was slurry washed with acetonitrile (61.9 Kg) at 15 to 25° C. for at least 30 minutes (target 30 to 60 minutes) before filtering at 15 to 25° C. The filter cake was washed with acetonitrile (19.2 Kg) at 15 to 25° C. The filter cake was slurry washed with acetonitrile (62.0 Kg) at 15 to 25° C. for at least 30 minutes (target to 60 minutes) before filtering at 15 to 25° C. The filter cake was washed with acetonitrile (18.5 Kg) at 15 to 25° C.

The solid was dried at up to 50° C. under a flow of nitrogen and analysed by KF for residual water content. Pass criterion ≤2.8% w/w water. The solid was analysed for residual DMSO levels by ¹H NMR. Pass criterion ≤12.2% w/w DMSO. The solid was analysed for residual acetonitrile levels by ¹H NMR. Pass criterion ≤2.0% w/w MeCN. The dried weight of the crude solid was measured, identified and analysed using 1H NMR spectroscopy and HPLC.

### Final Product: 1-ethyl-N-((1,2,3,5,6,7-hexahydro-s-indacen-4-yl)-carbamoyl)piperidine-4-sulfonamide (potassium salt) (14)

Output: 13.95 Kg

Yield: 80%

NMR purity: 97.3%

### Purification of 1-ethyl-N-((1,2,3,5,6,7-hexahydro-s-indacen-4-yl)-carbamoyl)piperidine-4-sulfonamide (potassium salt) (14)

Crude 1-ethyl-N-((1,2,3,5,6,7-hexahydro-s-indacen-4-yl)carbamoyl)piperidine-4-sulfonamide (potassium salt) (14) (14.71 Kg) was charged to a reaction vessel. Methanol (116.4 Kg) was charged to the vessel, the temperature was adjusted to 15 to 25° C. as required with stirring for 10 to 20 minutes (until a homogeneous cloudy solution with no lumps of solid present was formed). The solution was filtered through a 1 μm filter at to 25° C. The filter was washed with methanol (11.3 Kg) at 15 to 25° C. The solution was concentrated to ca. 44 L at 25 to 35° C. Acetonitrile (116.6 Kg) was charged to the mixture and the solution was concentrated to ca. 74 L at 25 to 35° C. Acetonitrile (58.7 Kg) was charged to the mixture and the mixture was concentrated to ca. 74 L at 35° C. The mixture was analysed for residual methanol content by ¹H NMR. Pass criterion 3.0% w/w methanol.

Acetonitrile (58.8 Kg) was charged to the vessel and the temperature was adjusted to 15 to 25° C. The slurry was aged for at least 1 hour (target 1 to 2 hours) at 15 to 25° C. and then filtered over 20 μm cloth at 15 to 25° C. The filter cake was twice washed with acetonitrile (23.9 Kg, 23.6 Kg) at 15 to 25° C.

The damp filter cake was analysed for residual phenol by HPLC. Pass criterion: 0.20% area phenol. The solid was dried at up to 50° C. under a flow of nitrogen for at least 2 hours and analysed for residual water content using KF. Pass criterion ≤2.0% w/w. Drying continued whilst the sample was being analysed.

The solid was analysed for residual acetonitrile by ¹H NMR. Pass criterion ≤0.2% w/w MeCN. The solid was analysed for residual DMSO by ¹H NMR. Pass criterion ≤0.4% w/w DMSO. The solid was analysed for residual solvent levels by GC. Pass criteria s 3750 ppm DMSO, ≤2250 ppm MeOH and ≤308 ppm MeCN.

### Final Product: 1-ethyl-N-((1,2,3,5,6,7-hexahydro-s-indacen-4-yl)-carbamoyl)piperidine-4-sulfonamide (potassium salt) (14)

Output: 14.42 Kg

Yield: 98%

HPLC purity: 99.5%

1. A process of preparing 1-ethyl-N-((1,2,3,5,6,7-hexahydro-s-indacen-4-yl)-carbamoyl)piperidine-4-sulfonamide or a salt thereof, comprising the step of contacting 1-ethyl-4-piperidinesulfonamide (A) with a 1,2,3,5,6,7-hexahydro-s-indacene derivative (B) in the presence of a solvent to obtain 1-ethyl-N-((1,2,3,5,6,7-hexahydro-s-indacen-4-yl)carbamoyl)piperidine-4-sulfonamide (C) or a salt thereof: 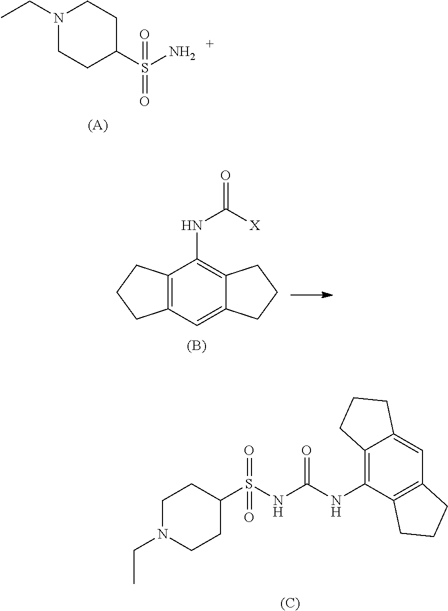
wherein X is a leaving group.

2. The process of claim 1, wherein X is Cl, Br, I, OR¹, SR¹, N(R¹)₂, OP(═O)(R¹)₂ or OP(R¹)₃⁺, wherein each R¹ is independently selected from a C₁-C₂₀ hydrocarbyl group, wherein each C₁-C₂₀ hydrocarbyl group may be straight-chained or branched, or be or include one or more cyclic groups, wherein each C₁-C₂₀ hydrocarbyl group may optionally be substituted with one or more oxo (═O) and/or one or more halo groups, and wherein each C₁-C₂₀ hydrocarbyl group may optionally include one or more heteroatoms independently selected from N, O and S in its carbon skeleton, or wherein any two R¹ together with the nitrogen or phosphorus atom to which they are attached may form a 3- to 16-membered heterocyclic group, wherein the heterocyclic group may be monocyclic, bicyclic or tricyclic, and wherein the heterocyclic group may optionally be substituted with one or more halo groups and/or one or more groups R^{X}, wherein each R^{X} is independently selected from a —CN, —OH, —NH₂, oxo (═O), ═NH or C₁-C₆ hydrocarbyl group, wherein each C₁-C₆ hydrocarbyl group may be straight-chained or branched, or be or include one or more cyclic groups, wherein each C₁-C₆ hydrocarbyl group may optionally be substituted with one or more halo groups, and wherein each C₁-C₆ hydrocarbyl group may optionally include one or more heteroatoms independently selected from N, O and S in its carbon skeleton.

3. The process of claim 1 wherein X is OR¹, wherein R¹ is selected from an aryl or heteroaryl group, wherein the aryl or heteroaryl group is monocyclic, bicyclic or tricyclic, wherein R¹ may optionally be substituted with one or more substituents independently selected from halo, —CN, —OH, —NO₂, —NH₂, —R¹⁰, —OR¹⁰, —NHR¹⁰, —N(R¹⁰)₂ or —N(O)(R¹⁰)₂, wherein each R¹⁰ is independently selected from a C₁-C₄ alkyl, C₁-C₄ haloalkyl, C₃-C₄ cycloalkyl or C₃-C₄ halocycloalkyl group, or any two R¹⁰ directly attached to the same nitrogen atom may together form a C₂-C₅ alkylene or C₂-C₅ haloalkylene group, and wherein R¹, including any optional substituents, contains from 1 to 20 carbon atoms.

4. The process of claim 1, wherein X is OPh.

5. The process of claim 1, wherein the solvent is dimethyl sulfoxide.

6. The process of claim 1, wherein the step of contacting 1-ethyl-4-piperidinesulfonamide (A) with the 1,2,3,5,6,7-hexahydro-s-indacene derivative (B) is performed in the presence of a base.

7. (canceled)

8. (canceled)

9. A process of preparing a 1,2,3,5,6,7-hexahydro-s-indacene derivative (B) or a salt thereof, the process comprising the step of converting 1,2,3,5,6,7-hexahydro-s-indacen-4-amine (D) into the 1,2,3,5,6,7-hexahydro-s-indacene derivative (B) or the salt thereof: 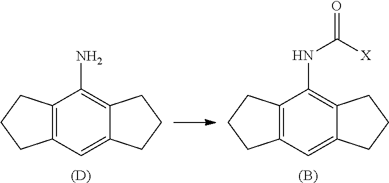
wherein X is a leaving group.

10. The process of claim 9, wherein the process comprises the step of contacting 1,2,3,5,6,7-hexahydro-s-indacen-4-amine (D) with reagent (E): 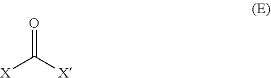
optionally in the presence of a base and/or a solvent, wherein X and X′ are leaving groups.

11. The process of claim 10, wherein:
(i) the solvent is tetrahydrofuran; and/or
(ii) the base is a tertiary amine.

12. The process of claim 9, wherein X is Cl, Br, I, OR¹, SR¹, N(R¹)₂, OP(═O)(R¹)₂ or OP(R¹)₃⁺, wherein each R¹ is independently selected from a C₁-C₂₀ hydrocarbyl group, wherein each C₁-C₂₀ hydrocarbyl group may be straight-chained or branched, or be or include one or more cyclic groups, wherein each C₁-C₂₀ hydrocarbyl group may optionally be substituted with one or more oxo (═O) and/or one or more halo groups, and wherein each C₁-C₂₀ hydrocarbyl group may optionally include one or more heteroatoms independently selected from N, O and S in its carbon skeleton, or wherein any two R¹ together with the nitrogen or phosphorus atom to which they are attached may form a 3- to 16-membered heterocyclic group, wherein the heterocyclic group may be monocyclic, bicyclic or tricyclic, and wherein the heterocyclic group may optionally be substituted with one or more halo groups and/or one or more groups R^{X}, wherein each R^{X} is independently selected from a —CN, —OH, —NH₂, oxo (═O), ═NH or C₁-C₆ hydrocarbyl group, wherein each C₁-C₆ hydrocarbyl group may be straight-chained or branched, or be or include one or more cyclic groups, wherein each C₁-C₆ hydrocarbyl group may optionally be substituted with one or more halo groups, and wherein each C₁-C₆ hydrocarbyl group may optionally include one or more heteroatoms independently selected from N, O and S in its carbon skeleton.

13. The process of claim 9, wherein X is OR¹, wherein R¹ is selected from an aryl or heteroaryl group, wherein the aryl or heteroaryl group is monocyclic, bicyclic or tricyclic, wherein R¹ may optionally be substituted with one or more substituents independently selected from halo, —CN, —OH, —NO₂, —NH₂, —R¹⁰, —OR¹⁰, —NHR¹⁰, —N(R¹⁰)₂ or —N(O)(R¹⁰)₂, wherein each R¹⁰ is independently selected from a C₁-C₄ alkyl, C₁-C₄ haloalkyl, C₃-C₄ cycloalkyl or C₃-C₄ halocycloalkyl group, or any two R¹⁰ directly attached to the same nitrogen atom may together form a C₂-C₅ alkylene or C₂-C₅ haloalkylene group, and wherein R¹, including any optional substituents, contains from 1 to 20 carbon atoms.

14. The process of claim 9, wherein X is OPh.

15. The process of claim 9, wherein X′ is Cl or Br.

16. A process comprising one or more steps selected from:
(a) converting 4-hydroxy piperidine (F) to a N-protected-4-hydroxy piperidine (G): 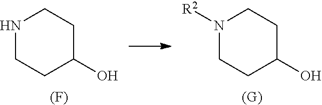
wherein R² is a nitrogen protecting group;
(b) converting a N-protected-4-hydroxy piperidine (G) to a N-protected-4-derivatised piperidine (H): 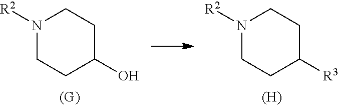
wherein R² is a nitrogen protecting group and R³ is a leaving group;
(c) converting a N-protected-4-derivatised piperidine (H) to a N-protected-4-(acylthio)-piperidine (I): 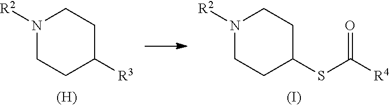
wherein R² is a nitrogen protecting group, R³ is a leaving group, and R⁴ is a C₁-C₂₀ hydrocarbyl group, wherein the C₁-C₂₀ hydrocarbyl group may be straight-chained or branched, or be or include one or more cyclic groups, wherein the C₁-C₂₀ hydrocarbyl group may optionally be substituted with one or more oxo (═O) and/or one or more halo groups, and wherein the C₁-C₂₀ hydrocarbyl group may optionally include one or more heteroatoms independently selected from N, O and S in its carbon skeleton;
(d) converting a N-protected-4-(acylthio)-piperidine (I) to a N-protected-4-(halosulfonyl)-piperidine (J): 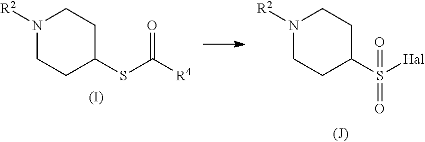
wherein R² is a nitrogen protecting group, R⁴ is a C₁-C₂₀ hydrocarbyl group, wherein the C₁-C₂₀ hydrocarbyl group may be straight-chained or branched, or be or include one or more cyclic groups, wherein the C₁-C₂₀ hydrocarbyl group may optionally be substituted with one or more oxo (═O) and/or one or more halo groups, and wherein the C₁-C₂₀ hydrocarbyl group may optionally include one or more heteroatoms independently selected from N, O and S in its carbon skeleton, and Hal is Cl or Br;
(e) converting a N-protected-4-(halosulfonyl)-piperidine (J) to a N-protected-4-piperidinesulfonamide (K): 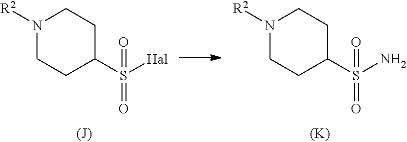
wherein R² is a nitrogen protecting group and Hal is Cl or Br; and
(f) converting a N-protected-4-piperidinesulfonamide (K) to 1-ethyl-4-piperidinesulfonamide (A): 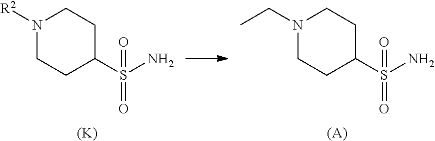
wherein R² is a nitrogen protecting group.

17. The process of claim 16, wherein R² is a nitrogen protecting group that may be removed by catalytic hydrogenolysis.

18. The process of claim 17, wherein the reaction step (f) comprises contacting the N-protected-4-piperidinesulfonamide (K) with acetonitrile or acetaldehyde in the presence of a catalyst and hydrogen gas, to obtain 1-ethyl-4-piperadinesulfonamide (A).

19. The process of claim 17, wherein the reaction step (f) comprises the steps of:
(i) contacting the N-protected-4-piperidinesulfonamide (K) with a first catalyst in the presence of hydrogen gas and a solvent to form an intermediate mixture comprising piperidine-4-sulfonamide and the solvent; and
(ii) contacting the intermediate mixture comprising piperidine-4-sulfonamide and the solvent with acetonitrile or acetaldehyde in the presence of a second catalyst and hydrogen gas, to obtain 1-ethyl-4-piperidine-sulfonamide (A).

20. The process of claim 16, wherein R² is —CH₂R²⁰ or —COOCH₂R²⁰, wherein R²⁰ is an aryl or heteroaryl group, wherein the aryl or heteroaryl group is monocyclic, bicyclic or tricyclic, wherein the aryl or heteroaryl group may optionally be substituted with one or more substituents independently selected from halo, —CN, —OH, —NO₂, —NH₂, —R²¹, —OR²¹, —NHR²¹, —N(R²¹)₂ or —N(O)(R²¹)₂, wherein each R²¹ is independently selected from a C₁-C₄ alkyl, C₁-C₄ haloalkyl, C₃-C₄ cycloalkyl or C₃-C₄ halocycloalkyl group, or any two R²¹ directly attached to the same nitrogen atom may together form a C₂-C₅ alkylene or C₂-C₅ haloalkylene group, and wherein R²⁰, including any optional substituents, contains from 1 to 20 carbon atoms.

21. The process of claim 16, wherein R² is —COOCH₂Ph.

22. The process of claim 16, wherein R³ is a sulphonate leaving group.

23. The process of claim 22, wherein reaction step (b) comprises contacting the N-protected-4-hydroxy piperidine (G) with a sulfonyl halide or a sulfonyl anhydride in the presence of a base to form the N-protected-4-derivatised piperidine (H).

24. The process of claim 16, wherein R⁴ is methyl.

25. The process of claim 16, wherein Hal is Cl.

26. The process of claim 16, wherein reaction step (a) comprises contacting the 4-hydroxy piperidine (F) with a nitrogen protecting group precursor in the presence of a base.

27. The process of claim 16, wherein reaction step (c) comprises contacting the N-protected-4-derivatised piperidine (H) with R⁴COS⁻.

28. The process of claim 16, wherein reaction step (d) comprises contacting the N-protected-4-(acylthio)-piperidine (I) with a halogenating agent in the presence of an acid and an aqueous solvent.

29. The process of claim 16, wherein reaction step (e) comprises contacting the N-protected-4-(halosulfonyl)-piperidine (J) with ammonia to form the N-protected-4-piperidinesulfonamide (K).

30. The process of claim 16, wherein the process is a process of preparing 1-ethyl-4-piperidinesulfonamide (A) or a salt thereof:

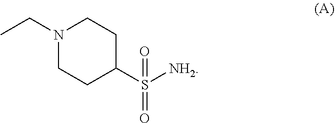

31. A compound selected from the group consisting of:
(i) a 1,2,3,5,6,7-hexahydro-s-indacene derivative (B) or a salt thereof: 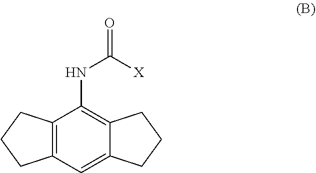
wherein X is a leaving group; or
(ii) an N-protected-4-hydroxy piperidine (G) or a salt thereof: 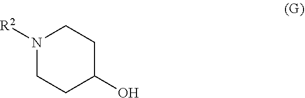
wherein R² is a nitrogen protecting group; or
(iii) an N-protected-4-derivatised piperidine (H) or a salt thereof: 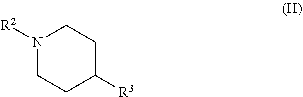
wherein R² is a nitrogen protecting group and R³ is a leaving group; or
(iv) an N-protected-4-(acylthio)-piperidine (I) or a salt thereof: 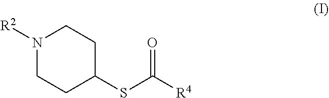
wherein R² is a nitrogen protecting group and R⁴ is a C₁-C₂₀ hydrocarbyl group, wherein the C₁-C₂₀ hydrocarbyl group may be straight-chained or branched, or be or include one or more cyclic groups, wherein the C₁-C₂₀ hydrocarbyl group may optionally be substituted with one or more oxo (═O) and/or one or more halo groups, and wherein the C₁-C₂₀ hydrocarbyl group may optionally include one or more heteroatoms independently selected from N, O and S in its carbon skeleton; or
(v) an N-protected-4-(halosulfonyl)-piperidine (J) or a salt thereof: 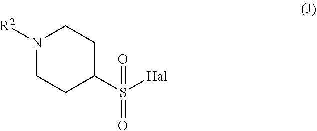
wherein R² is a nitrogen protecting group and Hal is Cl or Br; or
(vi) an N-protected-4-piperidinesulfonamide (K) or a salt thereof: 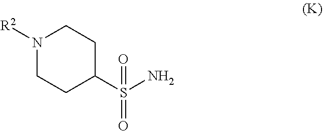
wherein R² is a nitrogen protecting group; or
(vii) 1-ethyl-4-piperadinesulfonamide (A) or a salt thereof:

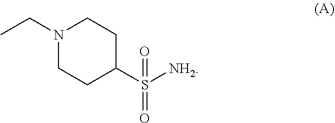

32. (canceled)
